(19) United States
(12) Patent Application Publication
(10) Pub. No.: US 2024/0158833 A1
(43) Pub. Date: May 16, 2024

(54) Compositions and Methods for Labeling Modified Nucleotides in Nucleic Acids

(71) Applicant: New England Biolabs, Inc., Ipswich, MA (US)

(72) Inventors: Laurence Ettwiller, Beverly, MA (US); Peter R. Weigele, Cambridge, MA (US); Weiwei Yang, Peabody, MA (US); Yan-Jiun Lee, Salem, MA (US); Ivan R. Correa, Jr., Hamilton, MA (US)

(73) Assignee: New England Biolabs, Inc., Ipswich, MA (US)

(21) Appl. No.: 18/546,896

(22) PCT Filed: Feb. 17, 2022
(86) PCT No.: PCT/US22/16743

§ 371 Date: Aug. 17, 2023

Related U.S. Application Data
(60) Provisional application No. 63/151,400, filed on Feb. 19, 2021.
(60) Provisional application No. 63/151,378, filed on Feb. 19, 2021.

(51) Int. Cl. C12Q 1/6806; C12N 9/10

(52) U.S. Cl. CPC C12Q 1/6806; C12N 9/1018; G01N 2333/91034

(57) ABSTRACT

Compositions, methods and kits are provided that describe a novel enzyme family called here a hydroxymethylcytosine carbamoyltransferase that transfers a carbamoyl phosphate substrate onto a hydroxymethylcytosine nucleoside triphosphate or a hydroxymethylcytosine in a nucleic acid. The carbamoyl phosphate substrate may be tagged with a chemically reactive group and optionally a functional group. This enables multiple uses of this enzyme and substrate for detecting nucleic acids with modified nucleotides, enriching for such nucleic acids, sequencing nucleic acids containing modified nucleotides, and for synthesizing oligonucleotides with various labels for various molecular biology applications including stabilizing RNA.

### BACKGROUND

There are approximately 2.6 billion cytosines in the human genome, and when both DNA strands are considered, 56 million of those are followed by guanines (CpGs). In mammalian genomes, 70% to 80% of CpG are modified (Sunagawa, et al. Science 348, 6237 (2015). Cytosines modified at the 5th carbon position with a methyl group result in 5-methylcytosine (5-mC) and oxidation of 5-mC results in the formation of 5-hydroxymethylcytosine (5-hmC). These modifications are important due to their impact on a wide range of biological processes including gene expression and development Chiu, et al. Clinical Metagenomics. Nat. Rev. Genet. 20, 341-355 (2019). Cytosine modifications are often linked with altered gene expression, for example, methylated cytosines are often associated with transcriptional silencing and are found at transcription start sites of repressed genes (Hu, et al. Nat. Commun. 4, 2151 (2013) or at repetitive DNA and transposons (Charlop-Powers, et al, Current Opinion in Microbiology, 19 70-75 (2014). Recently however, it has been reported that some genes can be activated by 3′ CpG island methylation during development (Cao, et al. Front. Microbiol. 8, 1829 (2017)). The ability to accurately detect 5-mC and 5-hmC can have profound implications in understanding biological processes and in the diagnosis of diseases such as cancer.

Driven by the response to bacterial Restriction-Modification systems, bacteriophage T4 developed glucosyltransferases (GT) that modified its genomic hydroxymethylcytosine (hmC) in double stranded DNA for its protection against bacterial host restriction endonucleases. This has provided a reagent that has been adopted for mapping and sequencing 5-mC and 5-hmC (see for example, Vaisvila, et al, BioRviv December 2019); Bacteriophage XP12 can fully methylate cytosine in its genome for the same reason.

Given the increased interest in analyzing, stabilizing and manipulating both RNA and DNA, it would be desirable to identify reagents that could add chemical groups with potentially active side groups to specific target nucleotides on single stranded DNA and on RNA in addition to double stranded DNA.

### SUMMARY

In general, a method for modifying hmC in a nucleic acid, is provided that includes (a) combining: an aliquot of a sample comprising nucleic acid obtained from a eukaryotic cell; a hydroxymethylcytosine carbamoyltransferase (hmC-CT), and a carbamoyl phosphate substrate to produce a reaction mixture, and (b) incubating the reaction mixture to modify the hmC in the nucleic acid with the carbamoyl substrate. The carbamoyl substrate may comprise a tag that contains a chemically reactive group that is capable of participating in an azide-alkyne cycloaddition reaction. Alternatively, the carbamoyl phosphate substrate may be untagged. The method may include additional steps such as sequencing the modified nucleic acid of (b) or an amplification product thereof in order to detect the modified hmC in the nucleic acid; determining the location of the modified hmC residues in the nucleic acid; separating the modified nucleic acid of (b) from unmodified nucleic acid using the modified hmC residues produced in (b); and/or visualizing the modified hmC in the modified nucleic acid of (b).

Additional features of the above described methods may include: treating the nucleic acid with a deaminase, before or after step (a); treating the nucleic acid with a methylcytosine (mC) dioxygenases before or after step (a), and/or treating the nucleic acid with a GT before or after step (a). Nucleic acids to be modified may be single-stranded or double-stranded. The modification of hmC by carbamoyl phosphate and hmC-CT may include ATP. In certain embodiments, methods may include (c) enzymatically labelling methyl cytosine in the nucleic acid with a substrate that differs from the carbamoyl substrate in (a); and (d) determining the presence and/or location of mC and hmC in the nucleic acid.

Where a tagged carbamoyl phosphate is used to modify the nucleic acid, the tag includes a chemically reactive group. Optionally, a functional group to the hmC in the nucleic acid of (b) via a reaction with the chemically reactive group. In one embodiment, wherein chemically reactive group enables a cycloaddition reaction. In another embodiment, the functional group includes an optically detectable label for example, a fluorescent label. Accordingly, the method may include (d) optically detecting the modified nucleic acids. In another embodiment, the functional group comprises a bulky group that can be detected by nanopore sequencing. Moreover, the method may include the step of (d) sequencing the modified nucleic acids by nanopore sequencing. In another embodiment, the functional group includes an affinity tag such as for example, biotin or desthiobiotin. The affinity tag may enable or facilitate enriching for target nucleic acids by for example, binding the nucleic acids to a support that binds to the affinity tag; washing the support; and releasing the nucleic acids that are bound to the support. The enriched nucleic acids may be released for sequencing where the presence and location of the hmC can be identified. The nucleic acids can be RNA or DNA and may be obtained from a eukaryotic cell that has been isolated from a biological fluid, from circulating nucleic acids in the biological fluid or from a cell lysate.

In general, a method is provided that includes combining: i. a sample comprising hydroxymethylcytosine ribonucleotides (hmrC) or hydroxymethylcytosine deoxyribonucleotides (hmdC); ii. a hmC-CT; and iii. a tagged carbamoyl phosphate, to produce a reaction mixture, and (b) incubating the reaction mixture to modify the hmrC or hmdC.

In general, a method is provided that includes: (a) combining: i. a pool of nucleoside triphosphates comprising hmrC or hmdC; ii. a hmC-CT; iii. a carbamoyl phosphate substrate; iv. a nucleic acid template; and v. a polymerase to produce a reaction mix, and (b) incubating the reaction mix to produce a nucleic acid product that contains modified cytosines. As appropriate, the polymerase may be an RNA polymerase, a DNA polymerase or a reverse transcriptase.

Embodiments of the method may be used to generate a nucleic acid product that is an aptamer, a DNA primer or DNA adapter, or an RNA selected from the group consisting of a messenger RNA, siRNA and a guide RNA. The reaction mix may be an in vitro transcription reaction mix.

For all the methods described above that utilize hmC-CT, the hmC-CT may have any of the following properties: an amino acid sequence that is least 80% identical to any of SEQ ID NO: 1, 29-47, 49 or 96-97; an amino acid sequence that is least 80% identical to any of SEQ ID NO: 1, 29-47, 49 or 96-97 and has a glutamine (Q) at a position corresponding to position 169 in SEQ ID NO:1; an amino acid sequence that is least 80% identical to any of SEQ ID NO: 1, 29-47, 49 or 96-97 and further comprising has at least one of a tyrosine (Y) at a position corresponding to position 170 in SEQ ID NO:1 or an alanine (A) corresponding to a position 171 in SEQ ID NO:1; an amino acid sequence that is least 80% identical to any of SEQ ID NO: 1, 29-47, 49 or 96-97 and does not have a serine (S), arginine (R), alanine (A), tyrosine (T) if adjacent to a serine (S), lysine (K), glycine (G), or glutamic acid (E) at a position corresponding to position 169 in SEQ ID NO: 1; one or more amino acids at positions in any of SEQ ID NO: 1, 29-47, 49 or 96-97 corresponding to amino acids selected from the group consisting of: asparagine (N) corresponding to position 393 in SEQ ID NO: 1, valine (V) or phenylalanine (F) corresponding to position 395 in SEQ ID NO: 1, threonine (T) corresponding to position 409 in SEQ ID NO: 1, aspartic acid (D) or Proline (P) corresponding to position 416 in SEQ ID NO: 1, asparagine (N) corresponding to position 428 in SEQ ID NO: 1, and methionine (M) corresponding to position 434 in SEQ ID NO:1; two or more residues at positions in any of SEQ ID NO: 1, 29-47, 49 or 96-97 corresponding to amino acids selected from the group consisting of: asparagine (N) corresponding to position 393 in SEQ ID NO: 1, valine (V) or phenylalanine (F) corresponding to position 395 in SEQ ID NO: 1, threonine (T) corresponding to position 409 in SEQ ID NO: 1, aspartic acid (D) or proline (P) corresponding to position 416 in SEQ ID NO: 1, asparagine (N) corresponding to position 428 in SEQ ID NO: 1, and methionine (M) corresponding to position 434 in SEQ ID NO:1; or three or more residues at positions in any of SEQ ID NO: 1, 29-47, 49 or 96-97 corresponding to amino acids selected from the group consisting of: asparagine (N) corresponding to position 393 in SEQ ID NO: 1, valine (V) or phenylalanine (F) corresponding to position 395 in SEQ ID NO: 1, threonine (T) corresponding to position 409 in SEQ ID NO: 1, aspartic acid (D) or Proline (P) corresponding to position 416 in SEQ ID NO: 1, asparagine (N) corresponding to position 428 in SEQ ID NO: 1, and methionine (M) corresponding to position 434 in SEQ ID NO:1.

In general, a composition comprising: a tagged carbamoyl phosphate having the formula

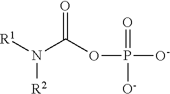

wherein: (i) the R1 and R2 in Formula 1 independently of each other may be an H or a tag (T) comprising a chemically reactive group (C) a functional group (F) and/or a linking group (L) where the linking group may be positioned between the carbamoyl group and the chemically reactive group and/or between the chemically reactive group and the label; and (ii) wherein the chemically reactive group (C) is selected from a succinimidyl ester, a maleimide, an amine, a thiol, an alkyne, or an azide, a carbonyl; a carboxyl; an active ester, e.g., a succinimidyl ester; a maleimide; an amine; a thiol; an alkyne, an azide; an alkyl halide; an isocyanate; an isothiocyanate; an iodoacetamide; a 2-thiopyridine; a 3-arylproprionitrile; a diazonium salt; an alkoxyamine; a hydrazine; a hydrazide; a phosphine; an alkene; a semicarbazone; an epoxy; a phosphonate; and a tetrazine.

The composition may include a functional group in the tag for example, an optically detectable moiety such as a fluorescent label exemplified by any of xanthene dyes, e.g. fluorescein and rhodamine dyes, such as fluorescein isothiocyanate (FITC), 6 carboxyfluorescein,6 carboxy-2′,4′,7′,4,7-hexachlorofluorescein (HEX), 6 carboxy 4′, 5′ dichloro 2′, 7′ dimethoxyfluorescein (JOE or J), N,N,N′,N′ tetramethyl 6 carboxyrhodamine (TAMRA or T), 6 carboxy X rhodamine (ROX or R), 5 carboxyrhodamine 6G (R6G5 or G5), 6 carboxyrhodamine 6G (R6G6 or G6), and rhodamine 110; or dyes exemplified by any of cyanine dyes, e.g. Cy3, Cy5 and Cy7 dyes; coumarins; benzimide dyes; phenanthridine dyes; ethidium dyes; acridine dyes; carbazole dyes; phenoxazine dyes; porphyrin dyes; polymethine dyes, cyanine dyes; BODIPY dyes or quinoline dyes.

The composition may include a functional group that is an affinity binding moiety selected from the group consisting of biotin and biotin analogs, avidin, protein A, maltose-binding protein, chitin binding domain, SNAP-tag® poly-histidine (New England Biolabs, Ipswich, MA), HA-tag, c-myc tag, FLAG-tag, GST, an epitope binding molecule such as an antibody and an oligonucleotide.

The composition may include a linking group (L), wherein the linking group is selected from the group consisting of: straight or branched chain alkylene group with 1 to 300 carbon atoms, a photocleavable linker, a saturated or unsaturated bicycloalkylene group, a divalent heteroaromatic group; and an oligonucleotide.

In one aspect, R1 or R2 in the composition has a chemically reactive group that is capable of participating in an azide-alkyne cycloaddition reaction for example, an azido or propargyl group The above described composition may include a hmC-CT that is optionally fused to an affinity binding domain or a DNA binding protein. The affinity binding domain fused to hmC-CT may include any of a biotin or desthiobiotin, streptavidin or avidin, maltose binding protein, methyl binding protein, chitin binding protein, SNAP-tag, antibody or fragment thereof, and Proteinase K or variant thereof. The fusion protein may include the tagged carbamoyl phosphate or a tagged carbamoyl methylcytosine (cmC) immobilized on a matrix such as a magnetic bead.

In one embodiment, the hmC-CT and optionally the tagged carbamoyl phosphate is lyophilized. In one embodiment, any of the compositions described above may include or be limited to a lyophilized hmC-CT. Any of the compositions described above may include or be limited to a lyophilized carbamoyl phosphate substrate.

In one embodiment, any of the compositions described above may include or be limited to a hmC-CT in a storage buffer containing at least 30%, 40% or 50% glycerol. The composition may further comprise an hmC-CT has at least 80% or 90% sequence identity to SEQ ID NO: 1, 29-47, 49 or 96-97. In general, a kit is provided that includes; (i) a hmC-CT, and (ii) a tagged carbamoyl phosphate. The tagged carbamoyl phosphate may include a chemically reactive group and optionally a functional group and a linker. The chemically reactive group in the tag can participate in an azide-alkyne cycloaddition reaction as desired. Examples of the chemically reactive group include an azido, an alkyne, a dibenzocyclooctyne (DBCO), or a tetrazine suitable for Click reactions. The tagged carbamoyl phosphate in the kit may include a functional group for example, an affinity tag or a detectable moiety. The kit may also contain in the same or separate containers, one or more reagents selected from carbamoyl phosphate, a TET family enzyme or mutant thereof, a GT, a deaminase, and a helicase. The kit may further include a reagent comprises an optically detectable label, a bulky group that can be detected by nanopore sequencing, an affinity tag, linked to a group that is capable of reacting with the tagged carbamoyl phosphate substrate, e.g., an azido or alkyne.

In general, a method for distinguishing hmC from mC in a nucleic acid molecule is provided that includes: (a) placing in a reaction mixture: the nucleic acid molecule; a hmC-CT and carbamoyl phosphate substrate; (b) modifying hmC in the nucleic acid molecule to form a cmC or tagged cmC; (c) detecting the cmC or tagged cmC in the nucleic acid molecule; and (d) distinguishing hmC from mC. The tagged carbamoyl phosphate in this method can include a functional group selected from a detectable moiety, an affinity binding moiety, a blocking moiety, and a bulky moiety. The nucleic acid may be chromosomal DNA and/or mRNA where the functional group in the tagged carbamoyl phosphate include a dye that is either a fluorescent or colored dye for detecting the location of hmC in vivo or in vitro. The method may further include sequencing the nucleic acid.

In general, a method is provided for obtaining nucleic acid modifying enzymes, that includes obtaining phage nucleic acid from an environmental sample from which phage particles have been enriched; identifying whether the phage nucleic acid has modified nucleotides; performing a contig analysis of the phage nucleic acid for sequences encoding enzymes capable of modifying the phage nucleic acid; and obtaining nucleic acid modifying enzymes.

In one embodiment, a method is provided for determining the presence of cytosine modifications in nucleic acid samples obtained from a biological fluid or a cell lysate where the biological fluid may include any of blood, urine, sputum, mucous, feces, and spinal fluid of human patients. For example, where the biological fluid is blood, it may contain low amounts of target nucleic acids such as for example, nucleic acids from exosomes or maternal and fetal nucleic acids.

The method may include (a) adding a carbamoyl group to any hmC in the nucleic acid samples; and (b) detecting the presence of cmC in the nucleic acid. The method may include adding a hmC-CT to the nucleic acid sample.

The carbamoyl phosphate in the method may be tagged with a functional domain on the carbamoyl phosphate that enables enrichment of the nucleic in the biological fluid or cell lysate by immobilizing the nucleic acids on a matrix such as a bead, a multi-well plastic dish or a paper by means of the cmC in the nucleic acid.

The nucleic acid can then be amplified and/or sequenced for determining the location of the hmC in the nucleic acid. Alternatively, the cmC can be detected using liquid chromatography-mass spectrometry.

In general, a method is provided for determining the location of modified cytosines (C) in a nucleic acid in a sample, that includes reacting an aliquot of the sample containing double stranded nucleic acid with (i) a GT for adding a sugar to 5-hmC, followed by (ii) a TET protein for oxidation of 5-mC and (iii) denaturing the nucleic acid into single strands and reacting the single stranded nucleic acid with a hmC-CT in the presence of a carbamoyl salt; and sequencing the glucosylated and carbamoylated single strand nucleic acid to determine which cytosines in the initial nucleic acid are unmodified or modified by a methyl or hydroxymethyl group.

In general, a method is provided for determining the location of modified cytosines in a nucleic acid in a sample, that includes: (a) reacting an aliquot of the sample in which the nucleic acid is single stranded with a hmC-CT and carbamoyl phosphate; (b) reacting the oxidized carbamoyl nucleic acid with a complementary single strand nucleic acid to form a double stranded DNA for reacting with TET protein; (c) permitting any methylated cytosines in the nucleic acid sample to be modified by adding GT; and (d) performing whole genome sequencing on double stranded nucleic acid to determine the location of 5-mC and 5-hmC in the nucleic acid. Step (a) of the method can be performed in in a single tube. The GT can be immobilized on a matrix for facilitating separation of the GT from the nucleic acid prior to addition of TET. An inhibitor of the GT can be added to the reaction prior to the addition of TET.

In general, a kit is described that contains a CT, and in the same or separate containers, one or more reagents selected from the group consisting of: carbamoyl phosphate, a TET family enzyme or mutant thereof, a GT; a deaminase, and a helicase.

In one embodiment, a composition is provided that includes a fusion protein wherein one portion of the fusion protein is a portion of a CT and a second portion of the fusion is an affinity binding domain or a DNA or RNA binding protein. In one aspect, the affinity binding domain is selected from the group consisting of biotin or desthiobiotin, maltose binding protein, methyl binding protein, chitin binding protein, SNAP-tag, antibody or fragment thereof, and Proteinase K or variant thereof. In another aspect, the fusion protein is immobilized on a matrix, for example, a magnetic bead.

The composition may be a lyophilized CT. Alternatively, the composition may be CT in a storage buffer that contains at least 30%, 40% or 50% glycerol. Optionally, any of the above compositions may be combined with an oligonucleotide for enhancing or depressing the activity of the CT in the presence of carbamoyl phosphate and a substrate nucleic acid or altering its specificity for modifying nucleotides in the substrate nucleic acid. In one aspect, the CT described herein has at least 80% or 90% sequence identity to SEQ ID NO:1.

In one embodiment, a composition is provided that includes a modified carbamoyl phosphate, wherein the modification is selected from one or more moieties consisting of a linker, a detectable moiety, an isolation tag, a blocking moiety, and a functional moiety. This composition may further include a CT.

In one embodiment, a method is provided for distinguishing 5-hmC from 5-mC in a nucleic acid molecule that includes (a) placing in a reaction mixture: the target nucleic acid molecule; a CT and carbamoyl phosphate (CP); and (b) modifying hmC in the nucleic acid molecule to form a 5-carbamoyloxymethylcytosine (5-cmC). The method may further include a step of detecting 5-carbamoyloxymethyldeoxyribocytosine (5-cmdC) or 5-carbamoyloxymethylribocytosine (5-cmrC) in the nucleic acid molecule. In one aspect of the method, the carbamoyl phosphate includes one or more moieties selected from the group consisting of a linker, a detectable moiety, an isolation tag, a blocking moiety, and a functional moiety.

In one aspect of the method, the nucleic acid having 5-cmC may be enriched by means of an affinity tag on one of: the carbamoyl phosphate, CT, or nucleic acid substrate. The nucleic acid in the reaction mixture may further be enriched by immobilization on a matrix.

In one aspect the nucleic acid, which may be DNA such as chromosomal DNA or RNA, is single stranded. Optionally examples of the method includes using dye tagged carbamoyl phosphate to detect the location of 5-hmC in vivo or in vitro where the dye is selected from a fluorescent dye or a color dye.

In one aspect, modified carbamoylated nucleic acids can be sequenced to determine the location of modified bases.

Another embodiment is a method directed to identifying novel nucleic acid modifying enzymes from a microbiome in an environmental sample. For example, the method may include the steps of: obtaining phage nucleic acid from an environmental sample from which phage particles have been enriched; identifying whether the phage nucleic acid has modified nucleotides; performing a contig analysis of the phage nucleic acid for sequences encoding enzymes capable of modifying the phage nucleic acid; and obtaining nucleic acid modifying enzymes.

Another embodiment is a method for determining the presence of nucleic acid modifications in low input samples obtained from a biological fluid or a cell lysate, wherein the method comprises: adding a carbamoyl group to hmC and detecting the presence of carbamoyl mC. The method may also include combining the nucleic acid from the low input sample with carbamoyl phosphate and CT. Examples of biological fluid include blood, urine, sputum, mucous, feces, and spinal fluid of human patients. Where the low input sample is from blood, the nucleic acids may be from exosomes, or in another example, may be maternal and fetal nucleic acids. The method may include enriching the low input nucleic in the biological fluid or cell lysate by immobilizing the nucleic acids on a matrix before or after adding the carbamoyl group to the hmC. Examples of a matrix include: a bead such as a magnetic bead, or a multi-well plastic dish or a paper. The present method may further include amplifying and/or sequencing the nucleic acids for detecting the presence of the cmC. The 5-cmdC in the nucleic acid may be detected by means of liquid chromatography-mass spectrometry. The present methods described herein may be used to determine a phenotype from the detected 5-cmdC.

In one embodiment, a method is provided that includes the steps of: (a) obtaining single stranded nucleic acid from a biological sample; (b) adding a carbamoyl group to some or all 5-hmC in the single strand nucleic acid sample; and optionally (c) oxidizing the 5-mC in the sample to 5-hmC and repeating (b). In one aspect, the single stranded nucleic acid from the biological sample is a low input DNA sample. In another aspect, the low input DNA is less than 100 ng, 10 ng, 1 ng or 100 pg. The single stranded nucleic acid from the biological sample may be single stranded DNA obtained from double stranded DNA that has been fragmented and denatured to form single strand DNA.

In one embodiment, the method described above may additionally include one or more of the following steps selected from the group consisting of: (i) adding a linking group to the carbamoyl phosphate for forming 5-cmdC or 5-cmrC in (b); (ii) ligating DNA adapters to the nucleic acid sample before (a), before or after (b) or before or after (c); (iii) adding an affinity tag to the linking group; enriching for the affinity tagged nucleic acid by affinity purification; (iv) amplifying the enriched DNA;

and (v) sequencing the carbamoylated nucleic acid.

In one embodiment, a method is provided for detecting 5-mC and 5-hmC in a single sequencing reaction wherein the method comprises reacting a nucleic acid in a sample sequentially or in parallel with a first and second blocking group such that 5-hmC is converted to a modified 5-hmC using one blocking group and 5-mC is modified with another blocking group optionally after oxidation of 5-mC so that both 5-mC and 5-hmC can be detected from a single sequence reaction. In one example, one blocking group is a carbamoyl group and another blocking group is glucose.

In another embodiment, a method is provided for determining the location of modified cytosines in a nucleic acid fragment in a sample, where the method includes: (a) reacting an aliquot of the sample containing double stranded nucleic acid with (i) a GT for adding a sugar to 5-hmC, followed by (ii) a TET protein for oxidation of mC and (iii) denaturing the nucleic acid into single strands and reacting the single stranded nucleic acid with a CT in the presence of a carbamoyl salt; and (b) sequencing the glucosylated and carbamoylated single strand nucleic acid to determine which Cs in the initial nucleic acid are modified by methyl or hydroxymethyl group. This method may be performed in a single tube.

The GT may be immobilized on a matrix for facilitating separation of the GT from the nucleic acid prior to addition of TET. Alternatively, or in addition, an inhibitor of the GT may be added prior to the addition of TET.

In another embodiment, a method is provided for determining the location of modified cytosines in a nucleic acid in a sample, comprising: (a) reacting an aliquot of the sample in which the nucleic acid is single stranded with a CT; (b) permitting any methylated cytosines in the nucleic acid sample to be oxidized by adding TET protein; (c) reacting the oxidized carbamoyl nucleic acid with a complementary single strand nucleic acid to form a double stranded DNA for reacting with GT; and (d) performing whole genome sequencing on double stranded nucleic acid to determine the location of 5-mC and 5-hmC in the nucleic acid.

In another embodiment, a synthetic oligonucleotide is provided containing one or more cmCs. The synthetic oligonucleotide may be an aptamer suitable for reversibly inhibiting enzyme activity of a target enzyme. The synthetic oligonucleotide may be designed for use in one or more of the following: splint ligation of a single stranded DNA or RNA fragments; a guide RNA for directing a cleavage of a nucleic acid by means of an enzyme and a guide or activator oligonucleotide; a leader sequence for RNA sequencing; an RNA or single strand DNA in a particle formulated for a vaccine; or a member of a sequencing array.

In another embodiment, a carbamoyl group is incorporated into a nucleic acid to facilitate whole molecule sequencing using sequencing platforms such as Oxford Nanopore and Pacific Biosystems that do not rely on amplifying the target nucleic acid molecule.

In another embodiment, a carbamoyl group may be used improve accuracy of sequencing of nucleic acids that contain polycytosine homopolymers within the nucleic acid. For example, some of the cytosines within the polycytosine homopolymers may be inefficiently methylated with a methylase and then oxidized to form hmC. The hmC may then be modified by a carbamoyl group using a CT and carbamoyl phosphate substrate as described herein.

In another embodiment, a carbamoyl group on the terminal nucleotide in an adapter or leader sequence can be used to signal the end of the reagent oligonucleotide sequence and the beginning of the target nucleic acid sequence for long nucleic acid sequencing in platforms such as Oxford Nanopore and Pacific Biosystems.

### BREIF DESCRIPTION OF FIGURES

FIG. 1 shows the methodology used to discover a new family of nucleotide modifying enzymes.

Meta Genotype-Phenotype Association (Meta GPA) relies on two cohorts, the case cohort composed of a group of organisms that share a specific phenotype and the control cohort composed of all organisms. Both cohorts were sequenced, de-novo, assembled into contigs and protein domains were annotated to contigs using automatic annotation pipelines. Protein domains significantly associated with case cohorts were compared to the control cohorts using phylogenetic relatedness that refines the annotation with phenotypic data; co-occurrence that allows to define functional units describing complete pathways with other associated domains; and residue associations that identifies critical regions/residues for phenotype differentiation.

These multilayer analyses effectively marked candidate protein domains related to the studied phenotype for later biological validation.

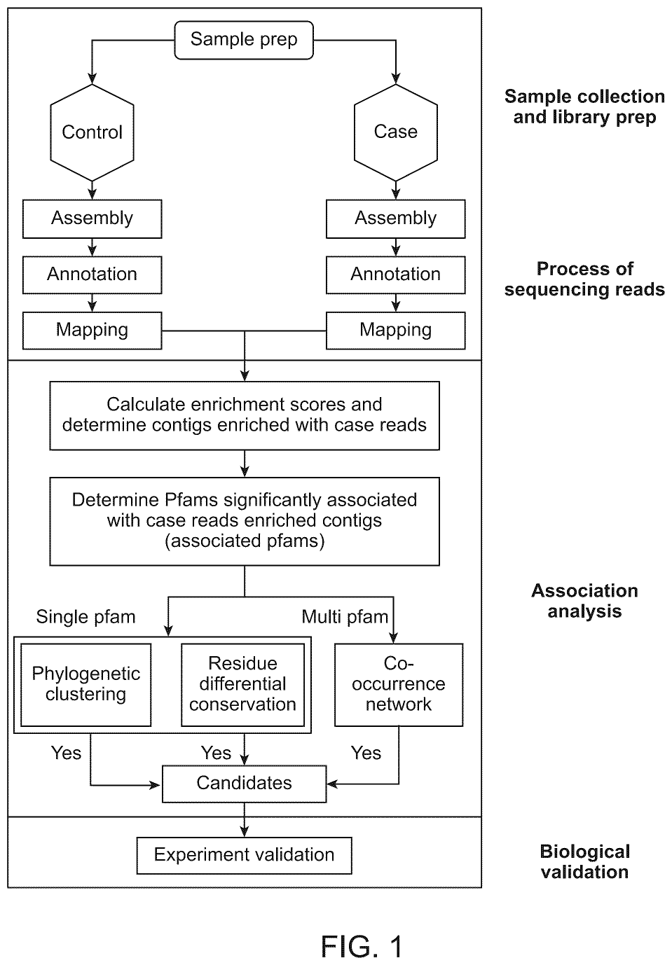

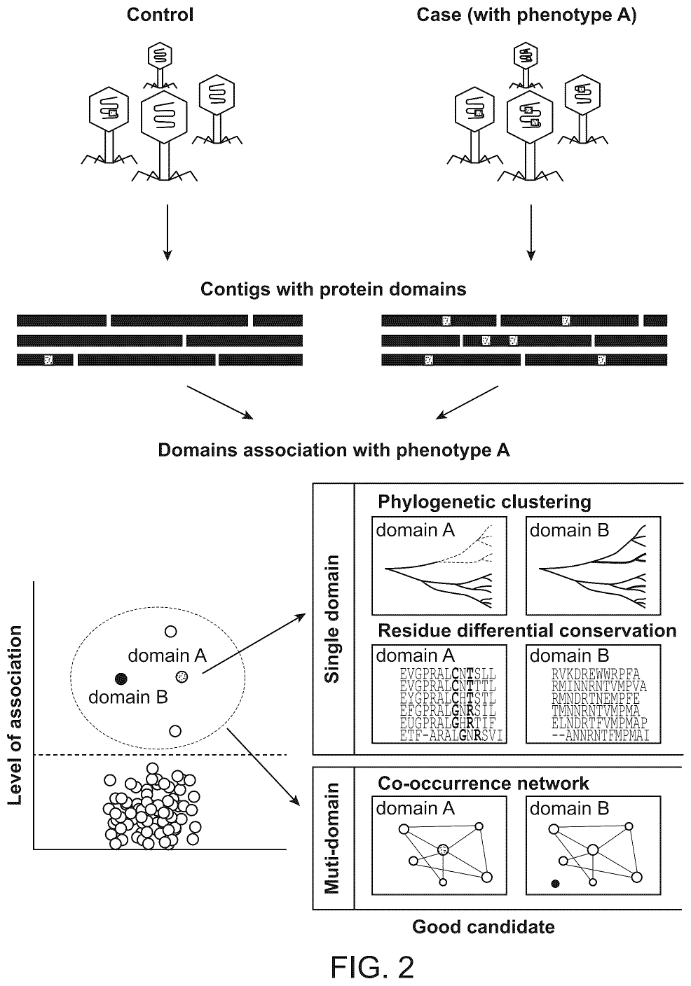

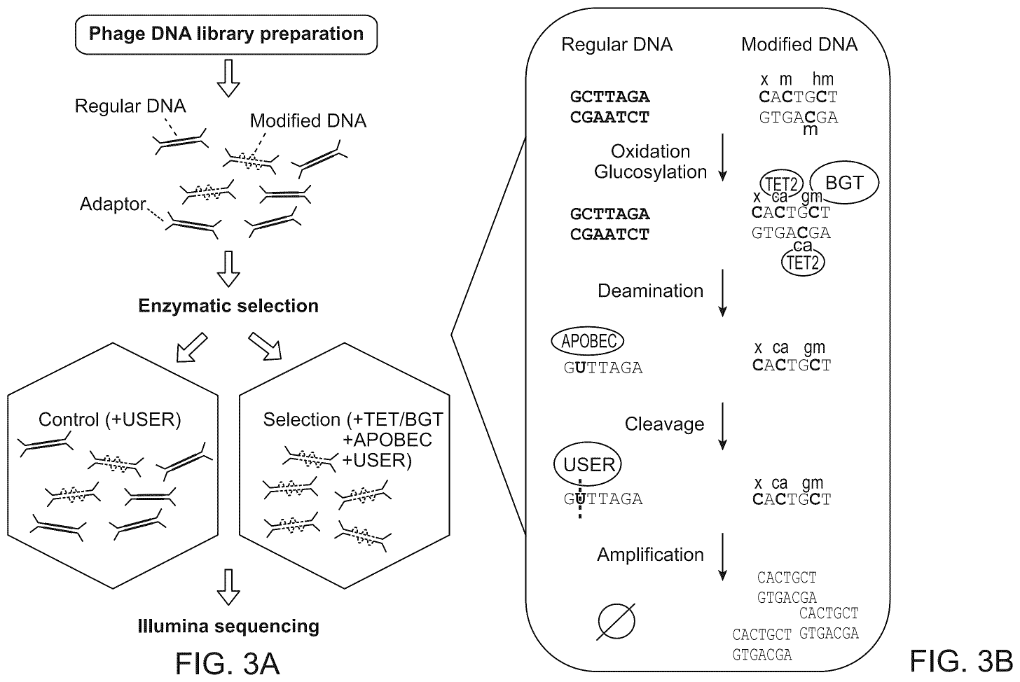

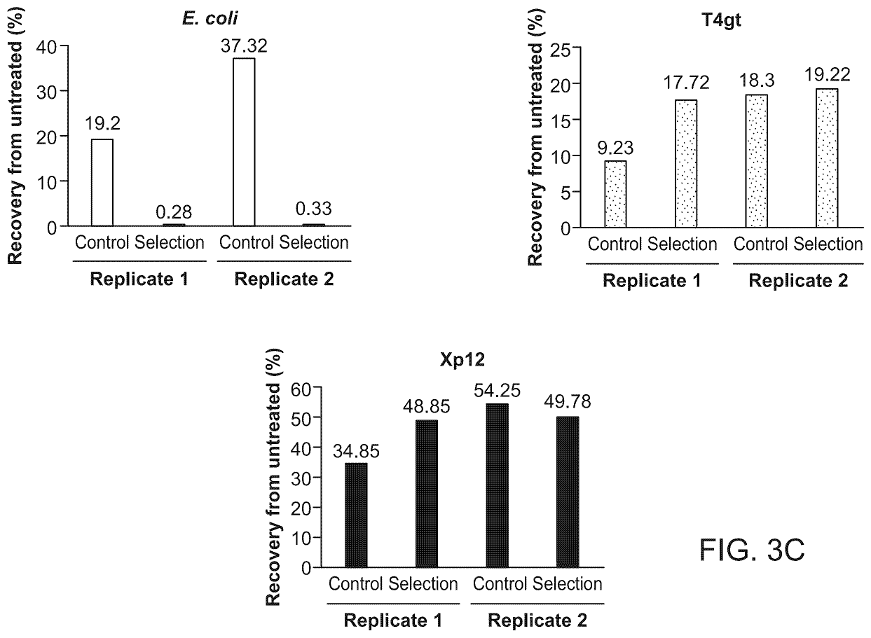

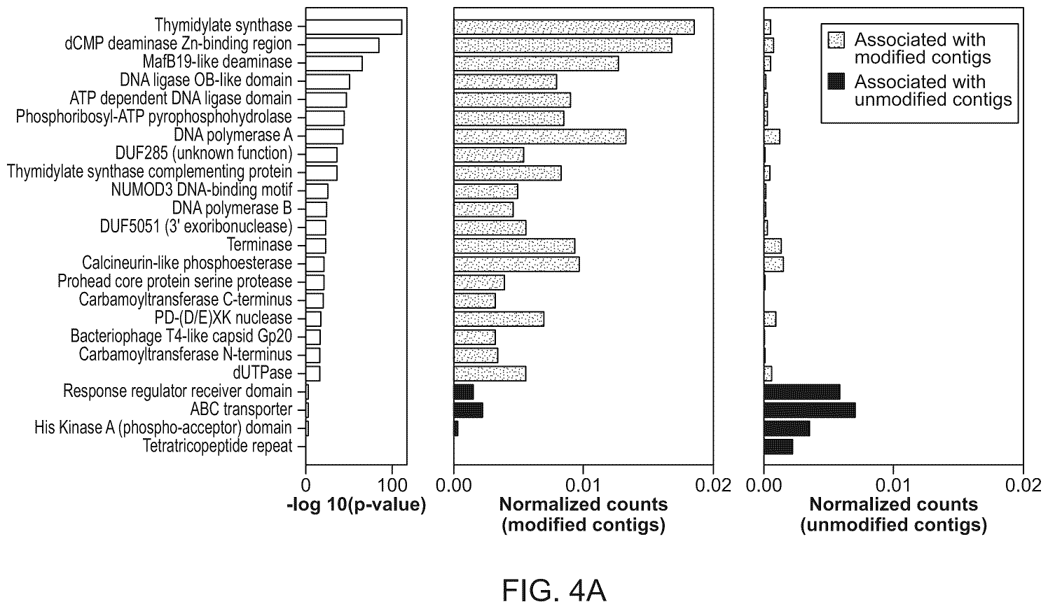

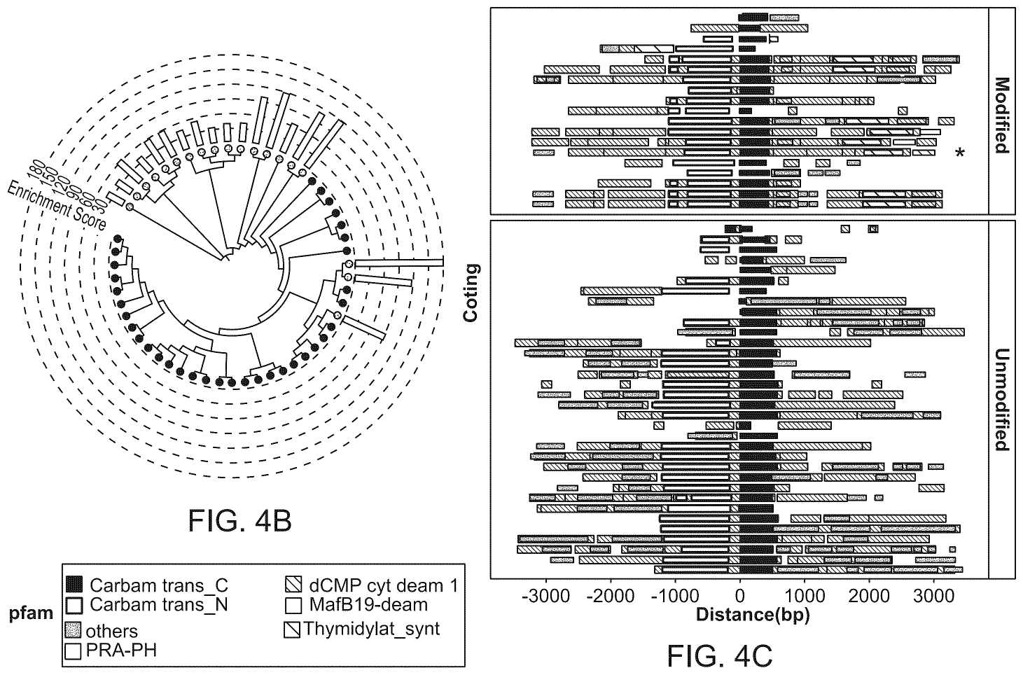

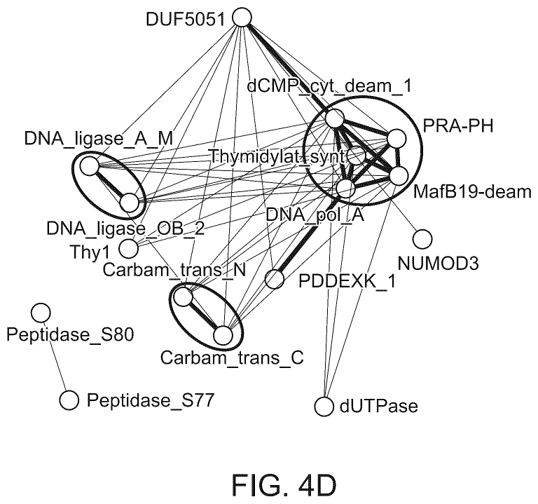

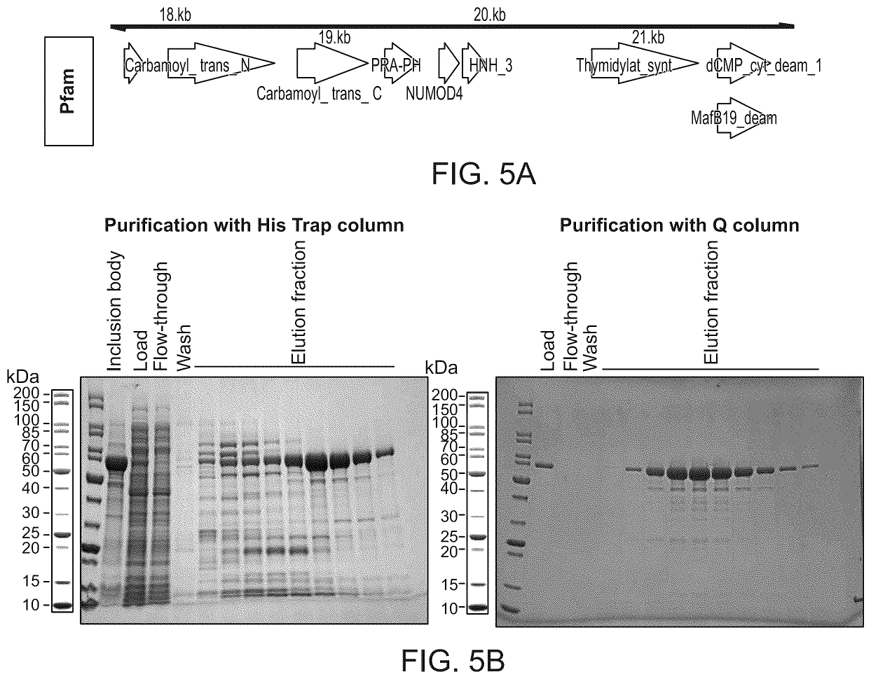

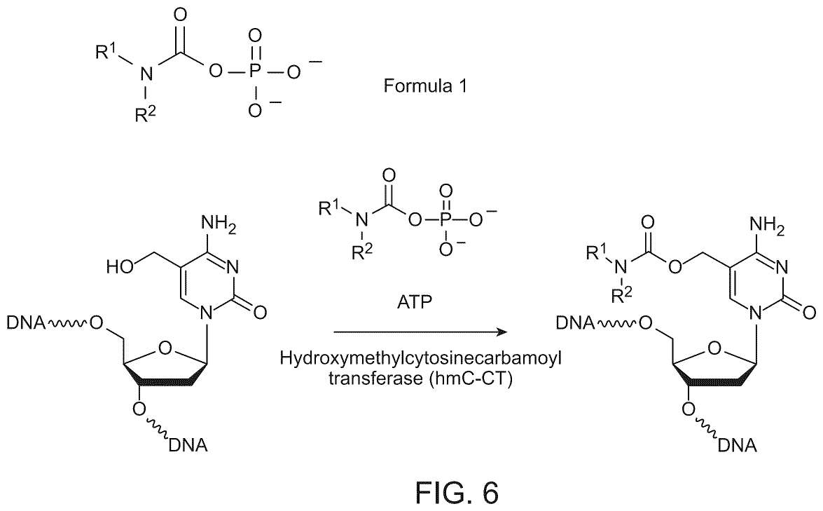

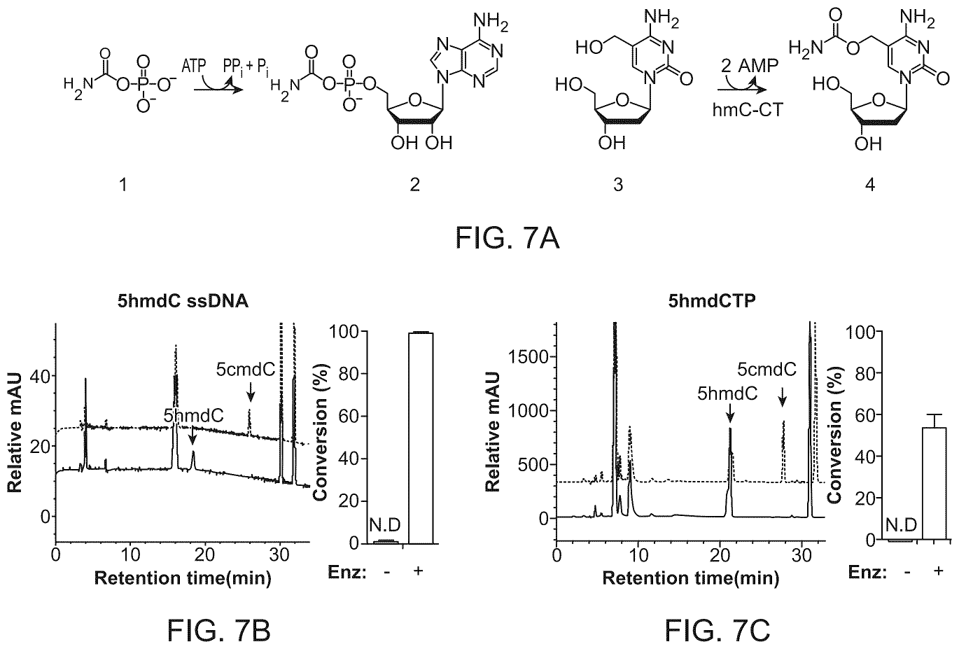

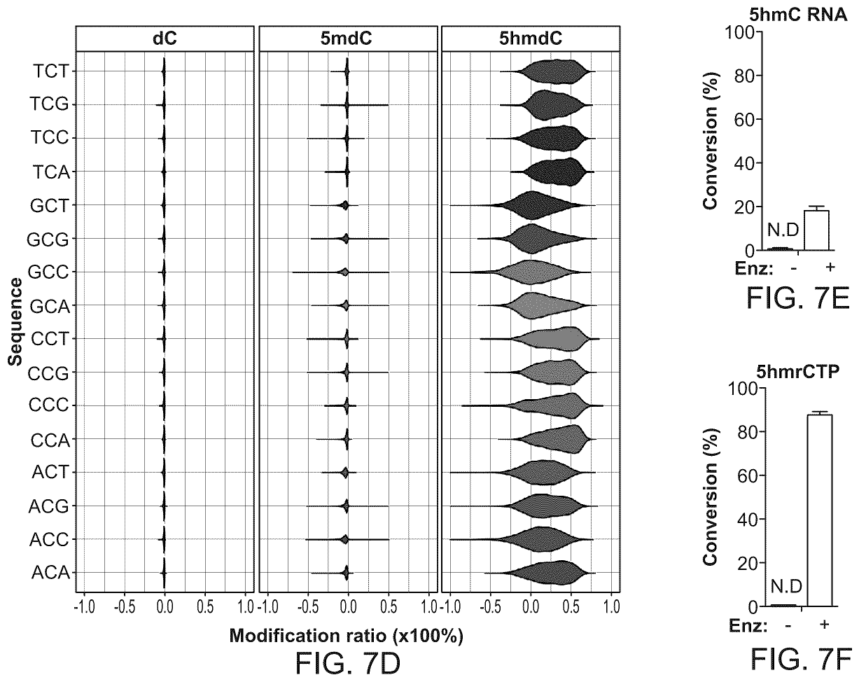

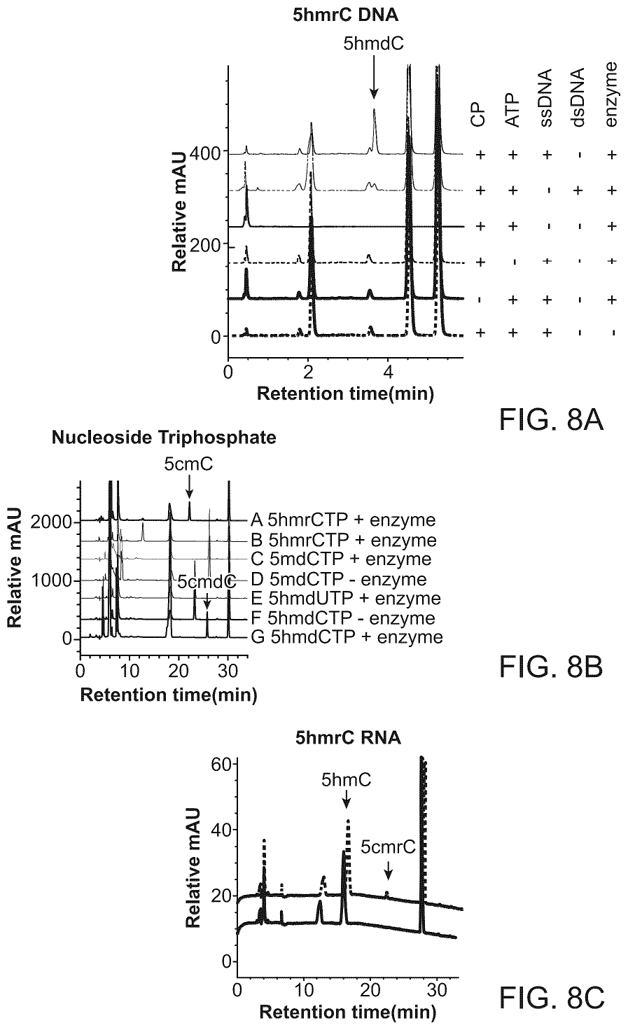

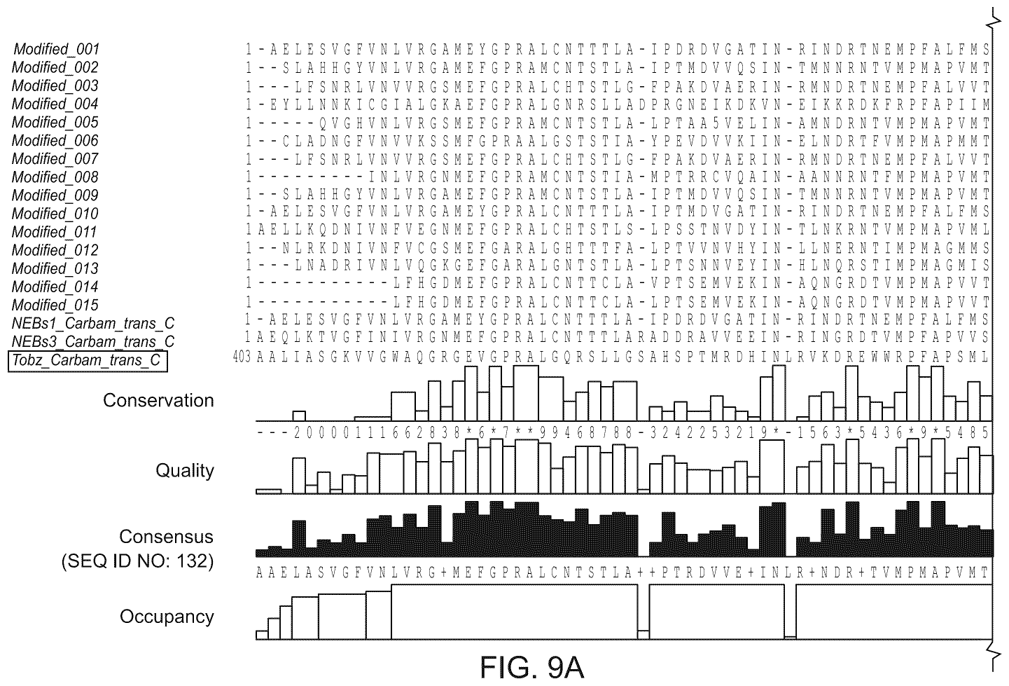

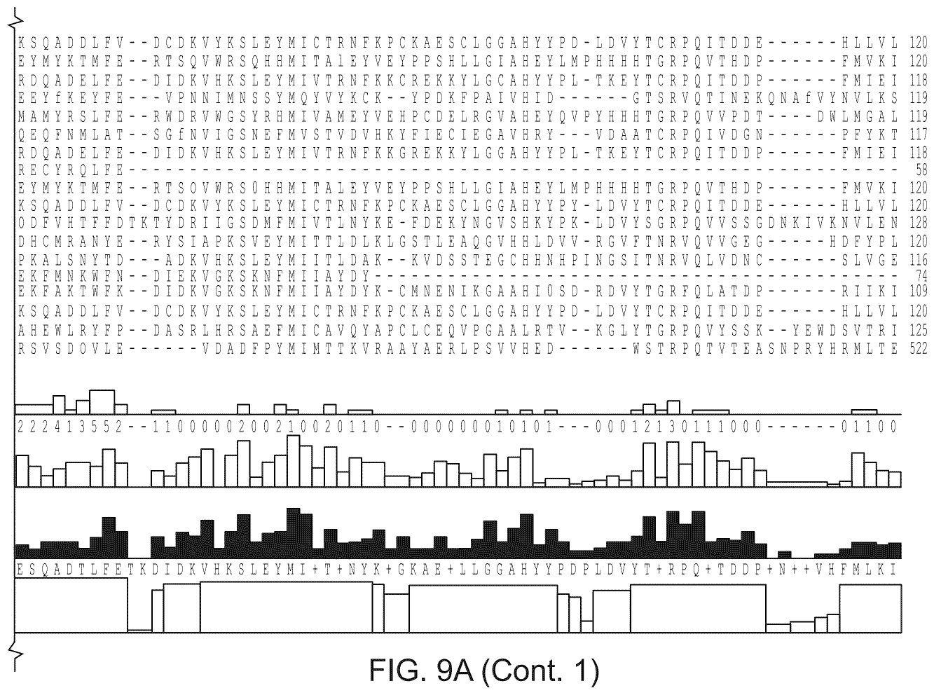

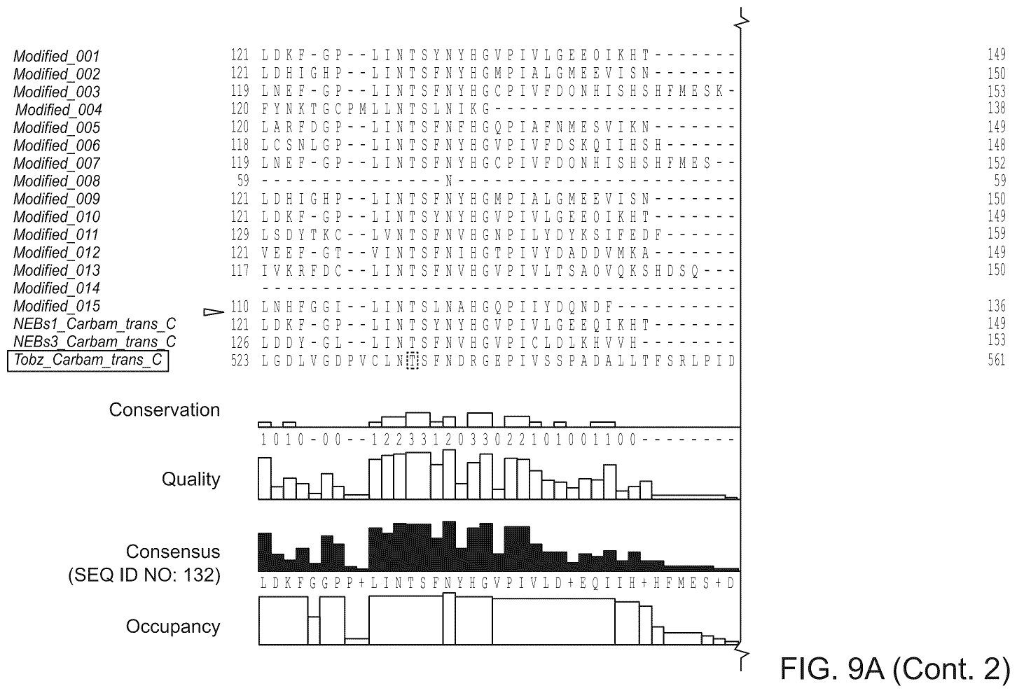

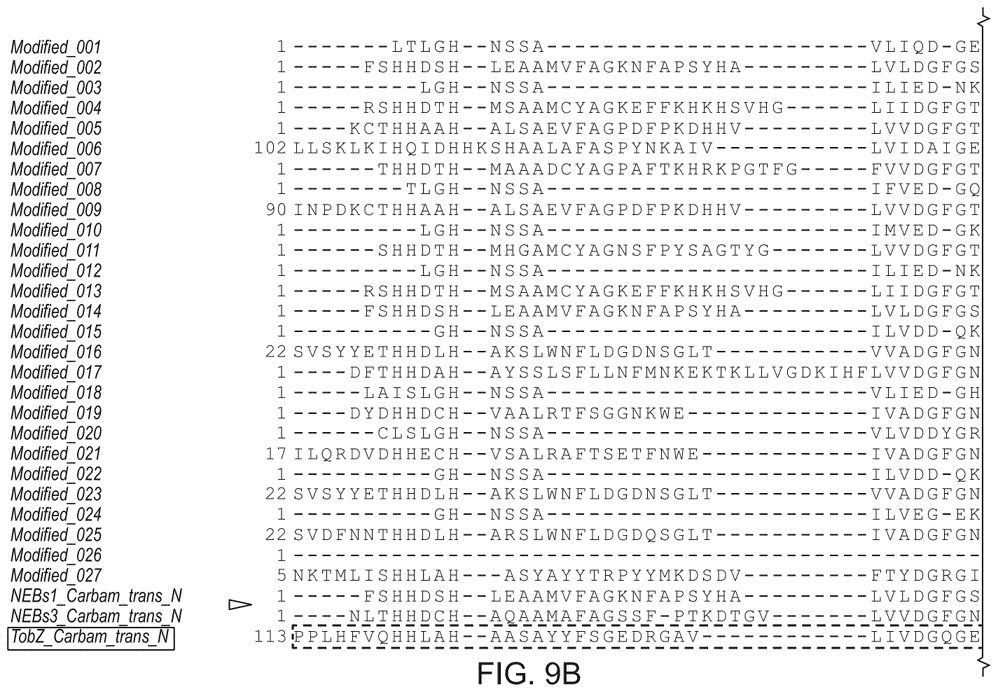

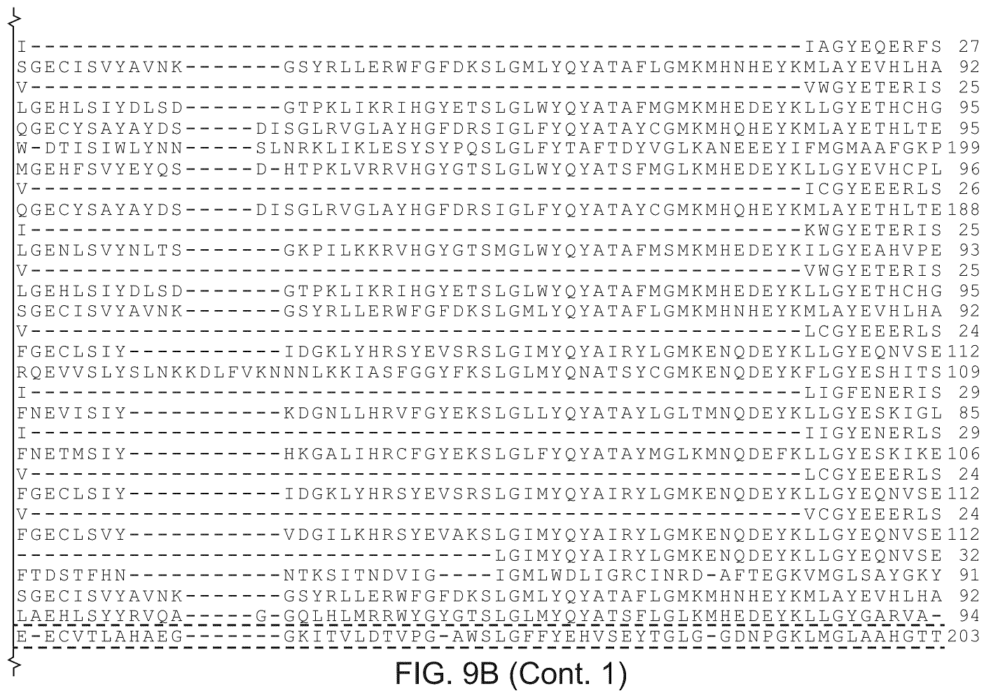

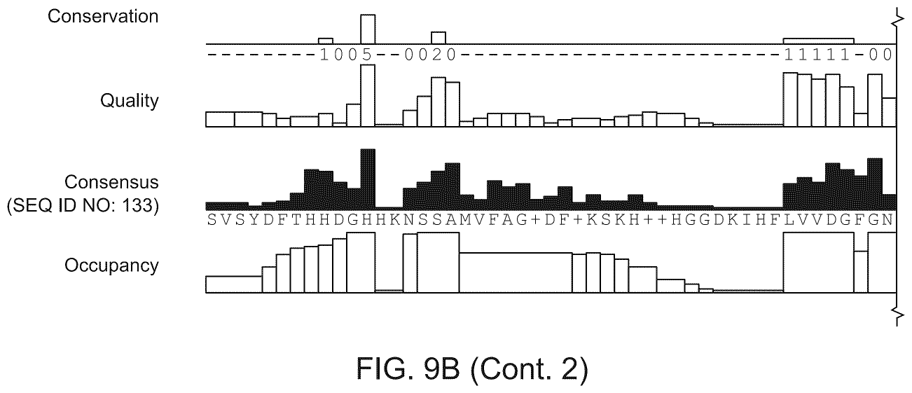

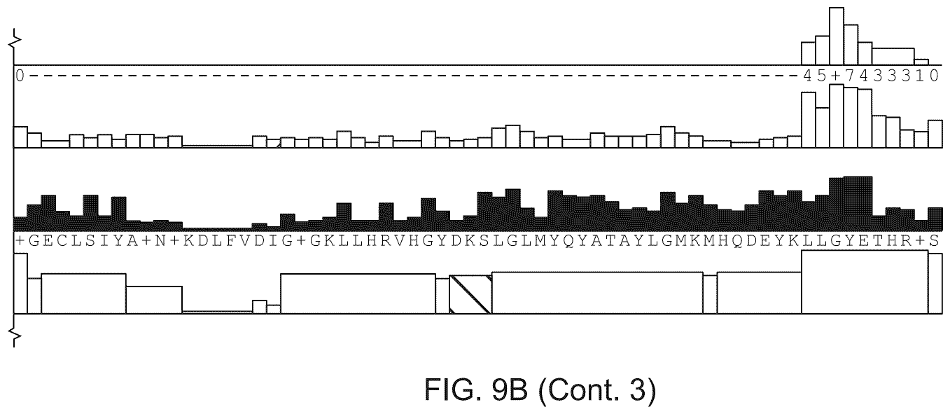

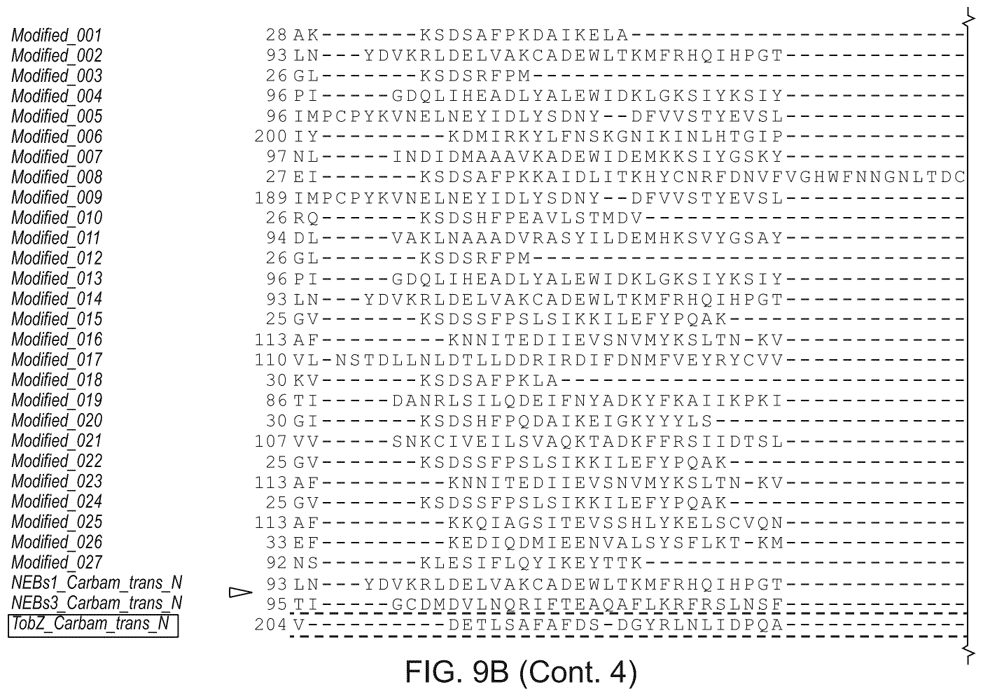

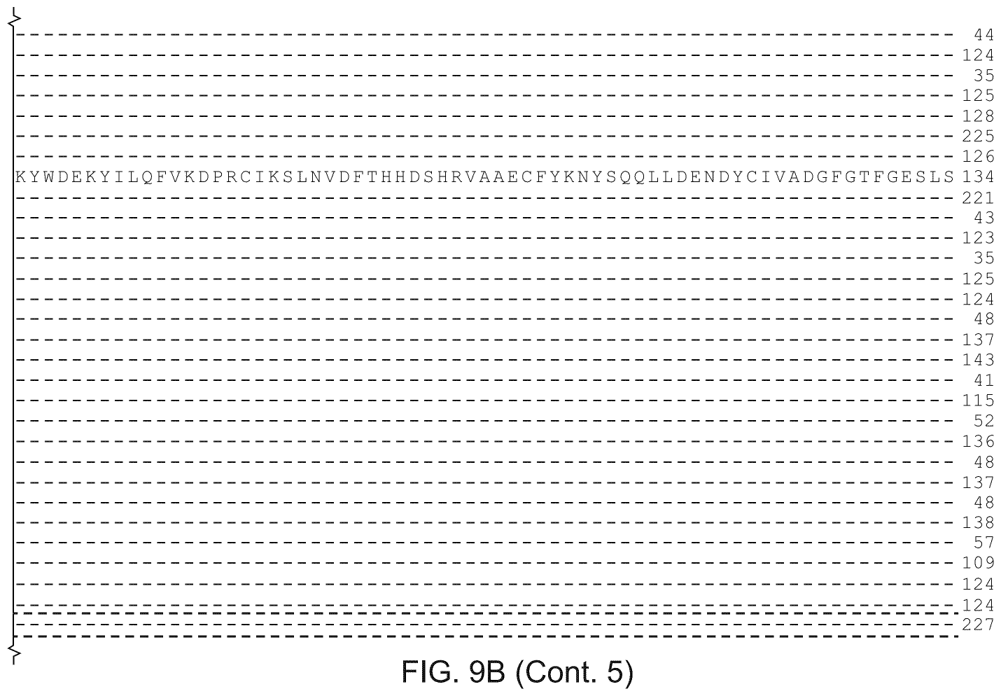

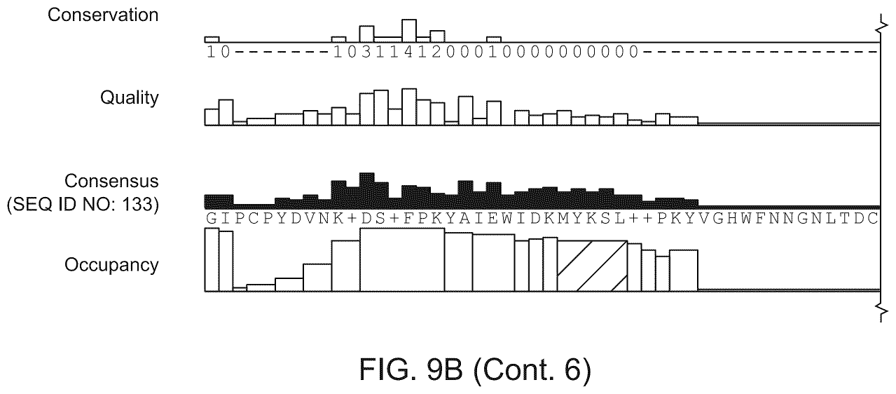

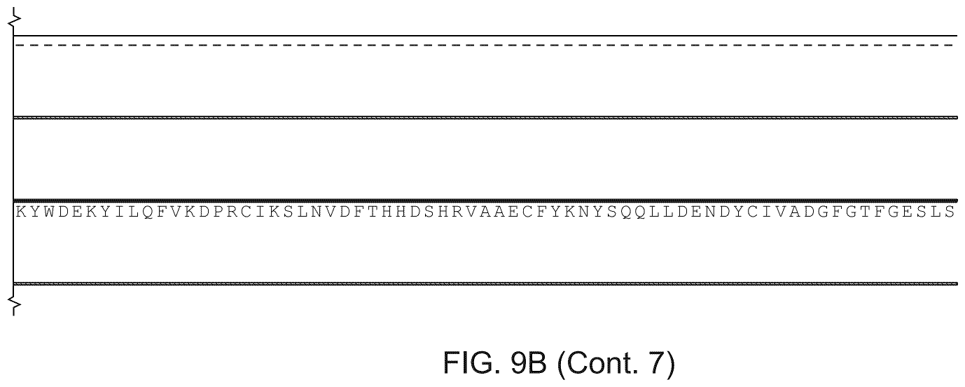

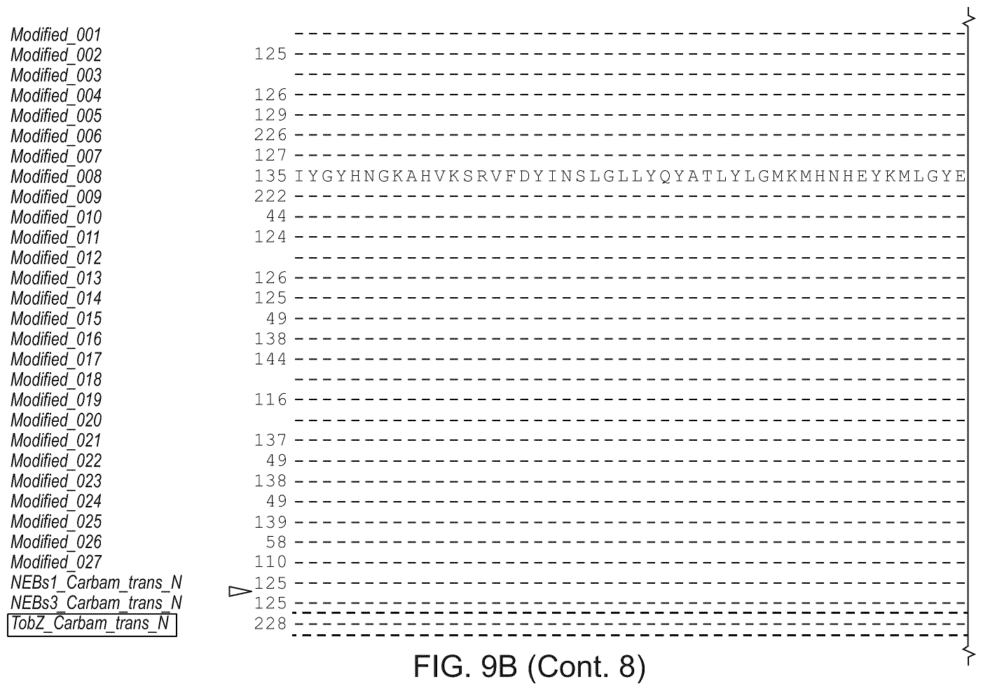

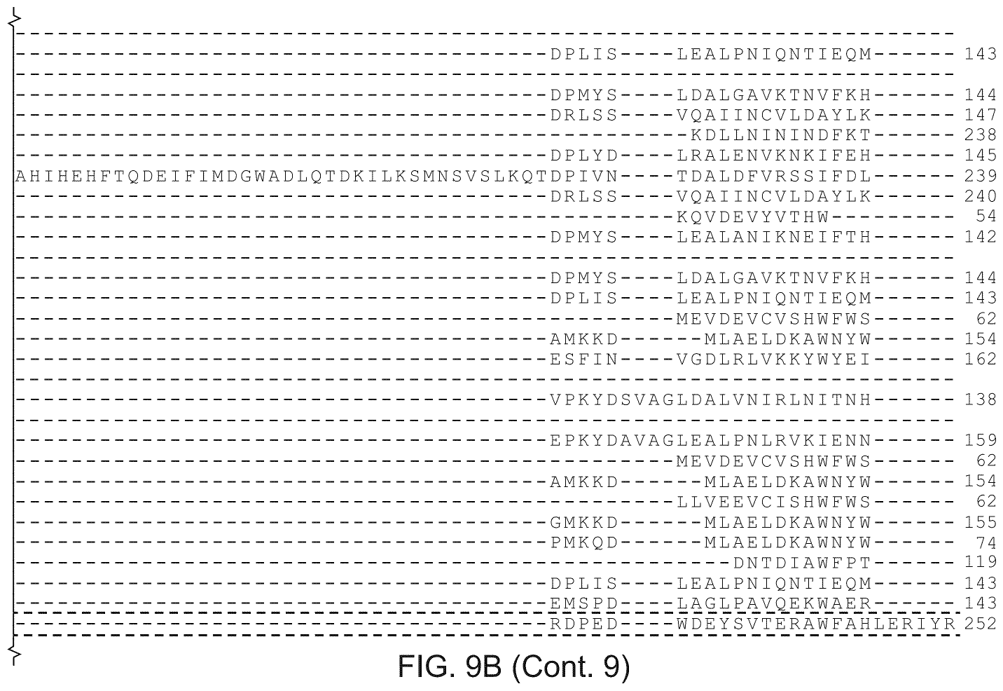

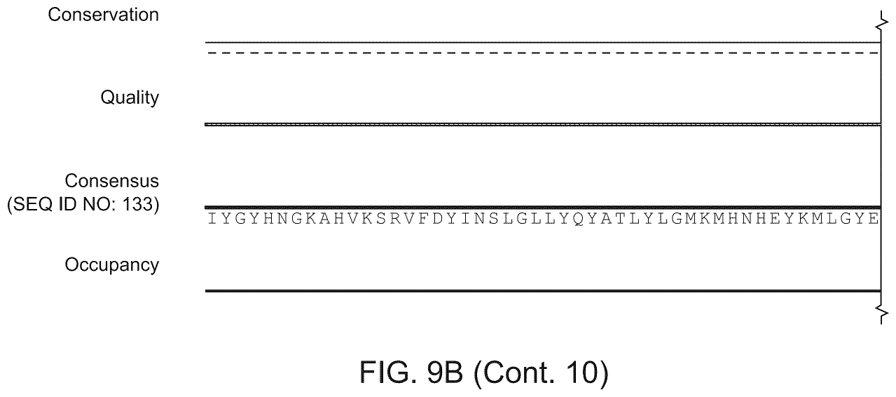

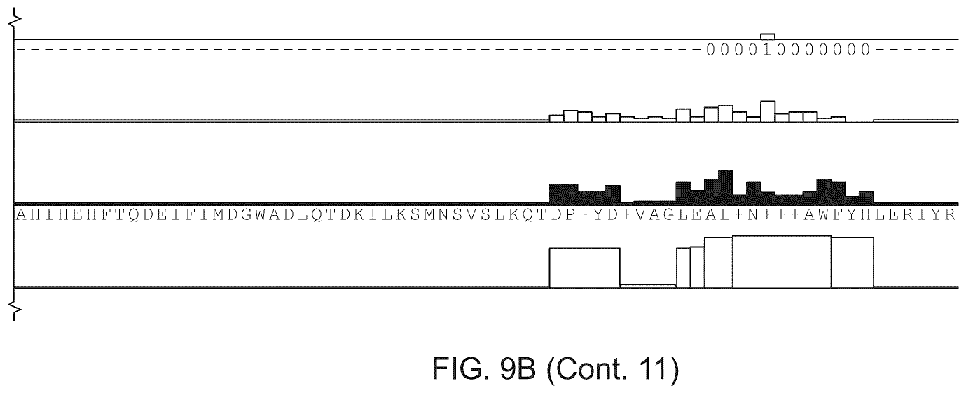

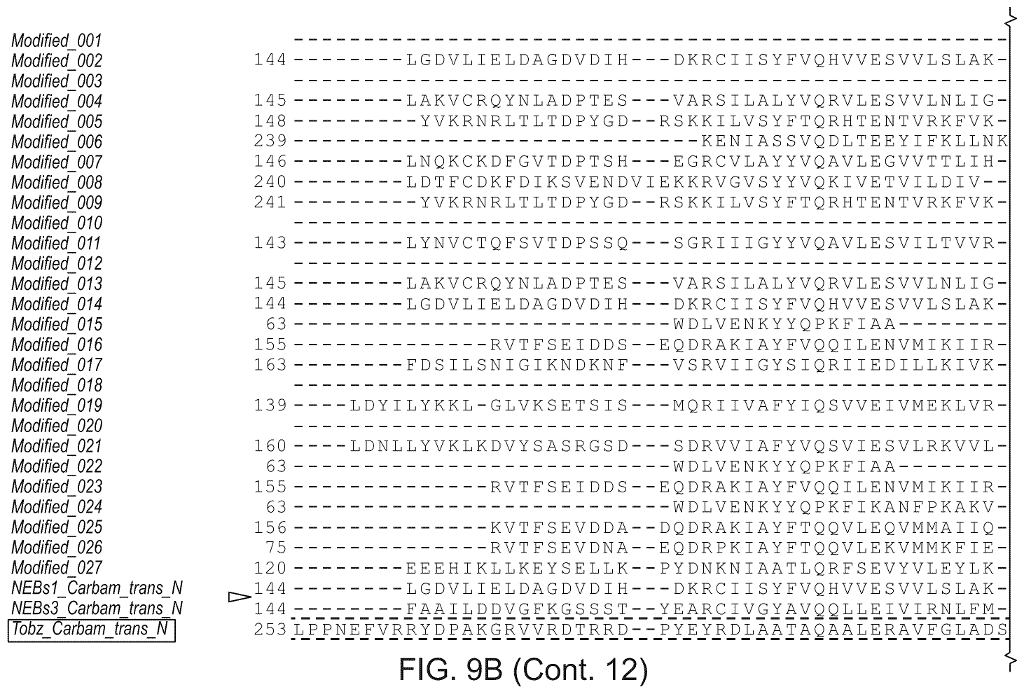

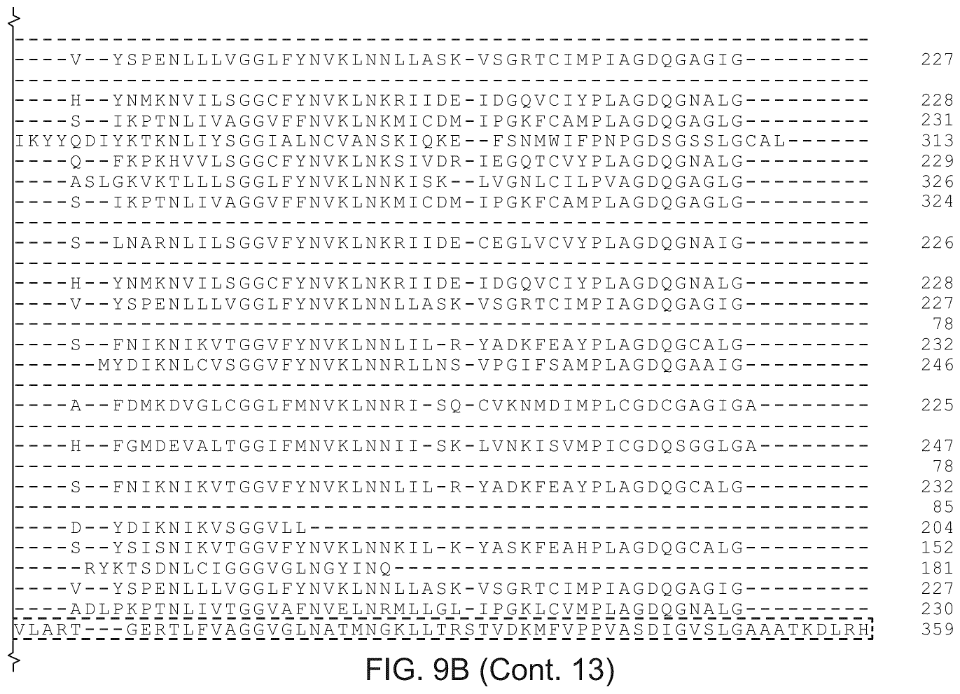

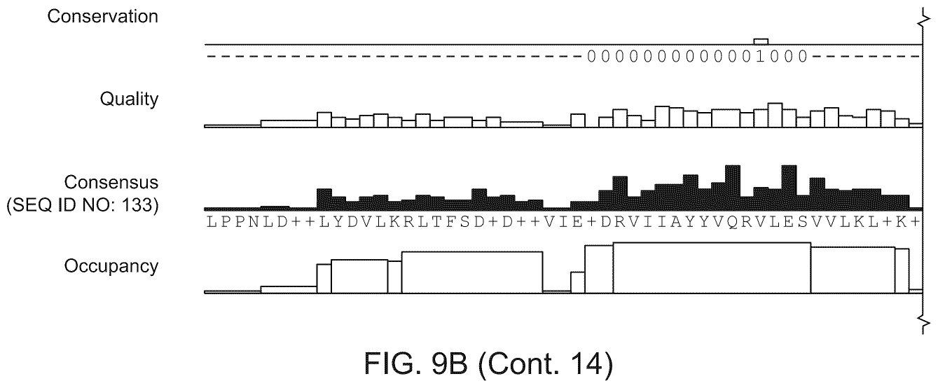

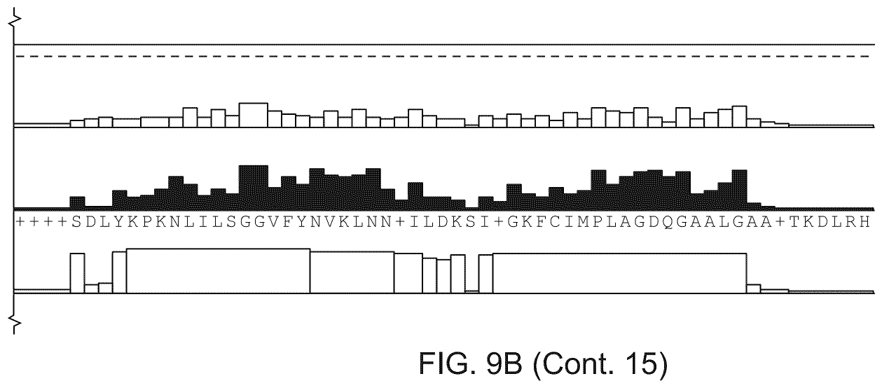

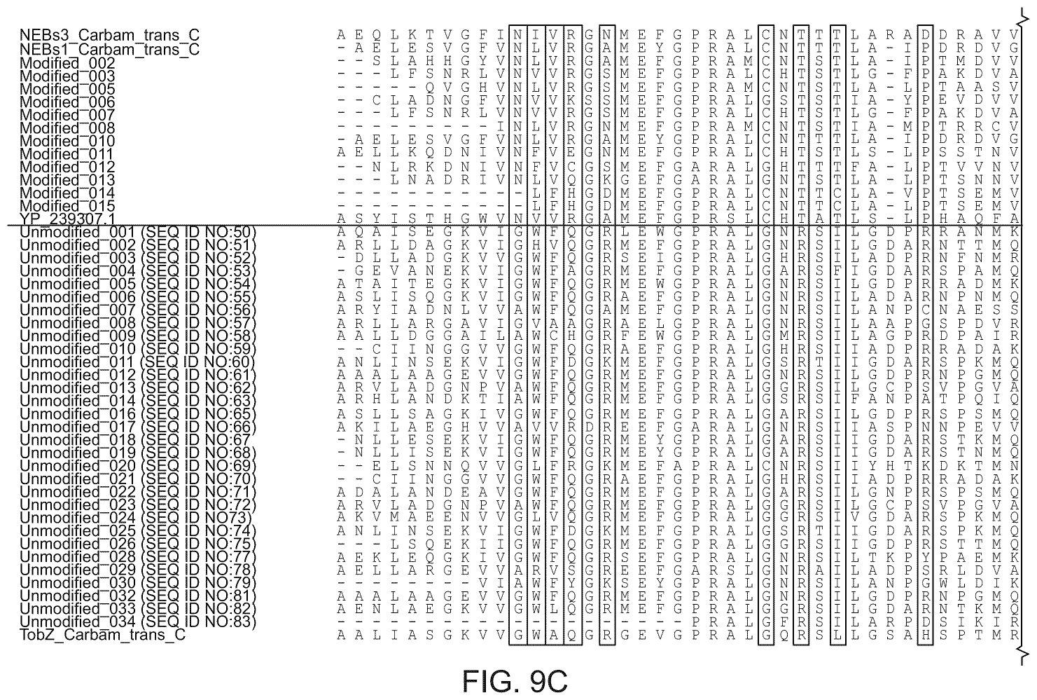

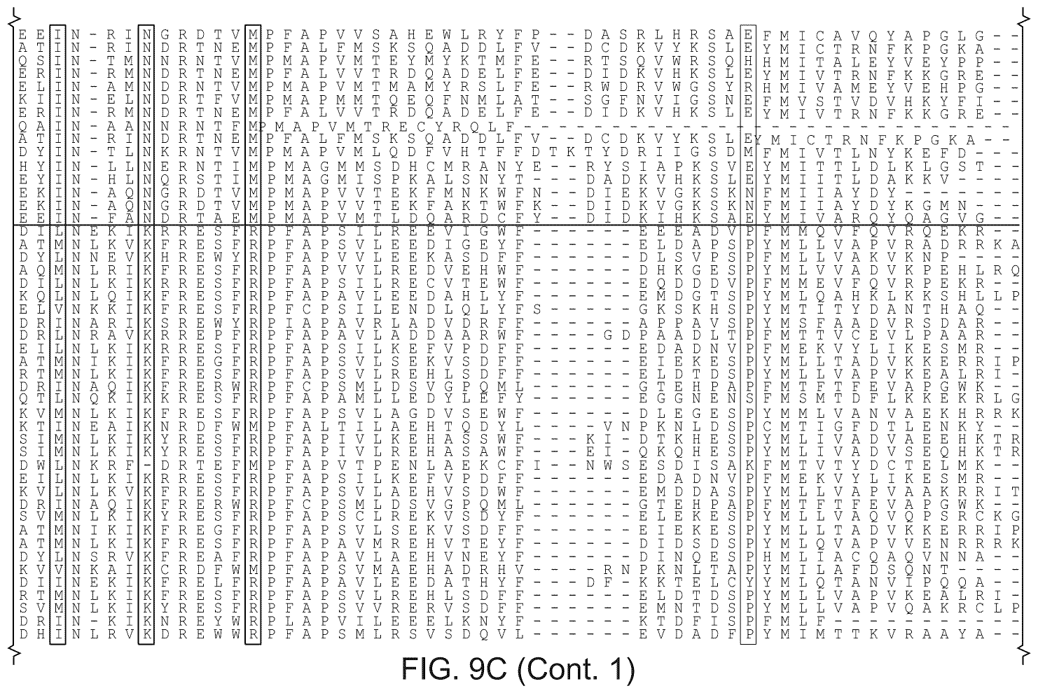

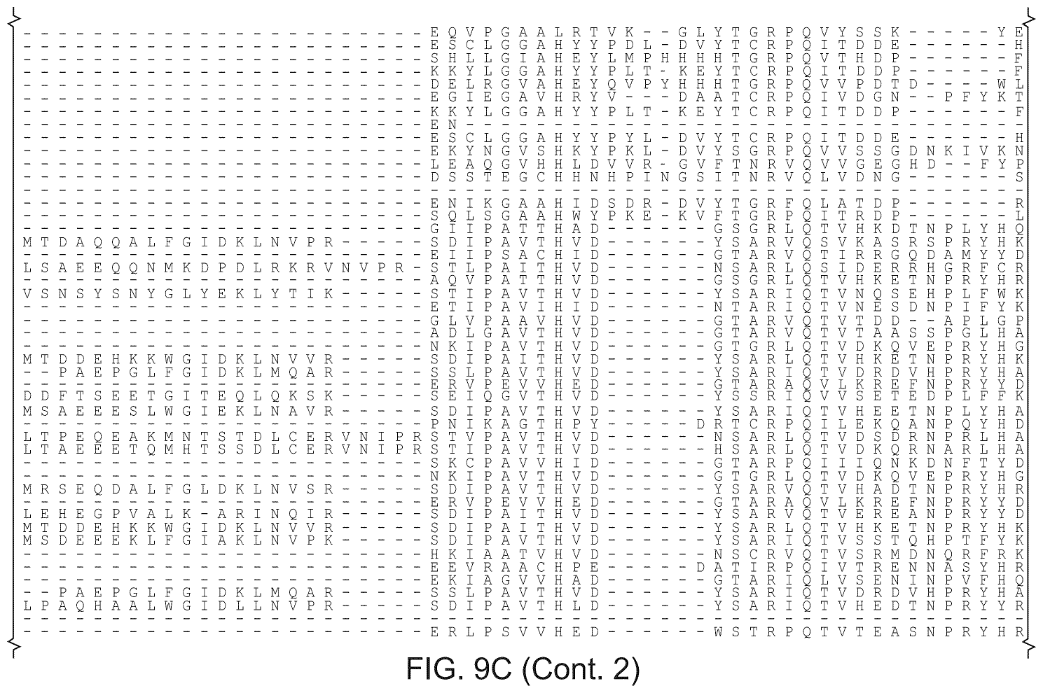

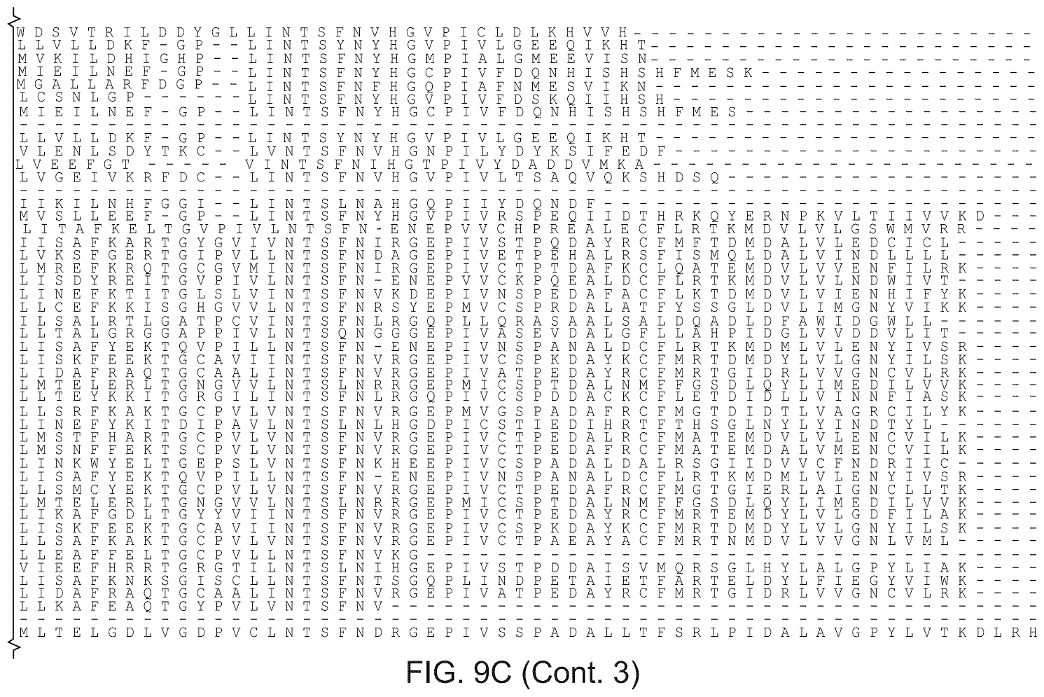

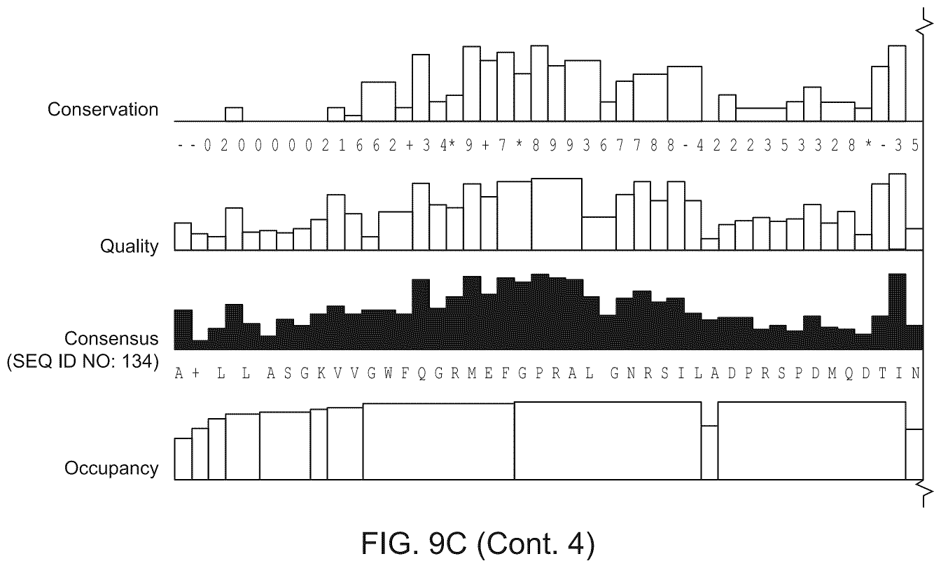

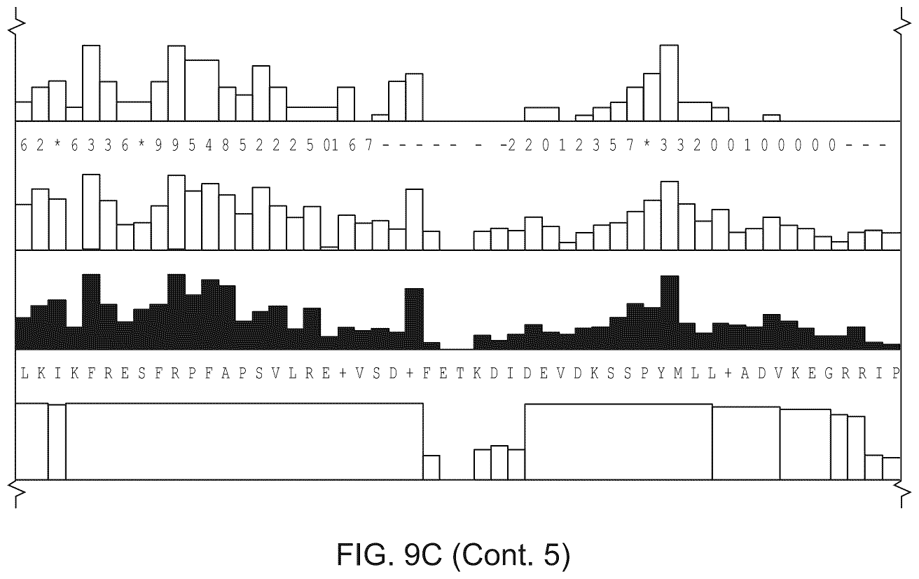

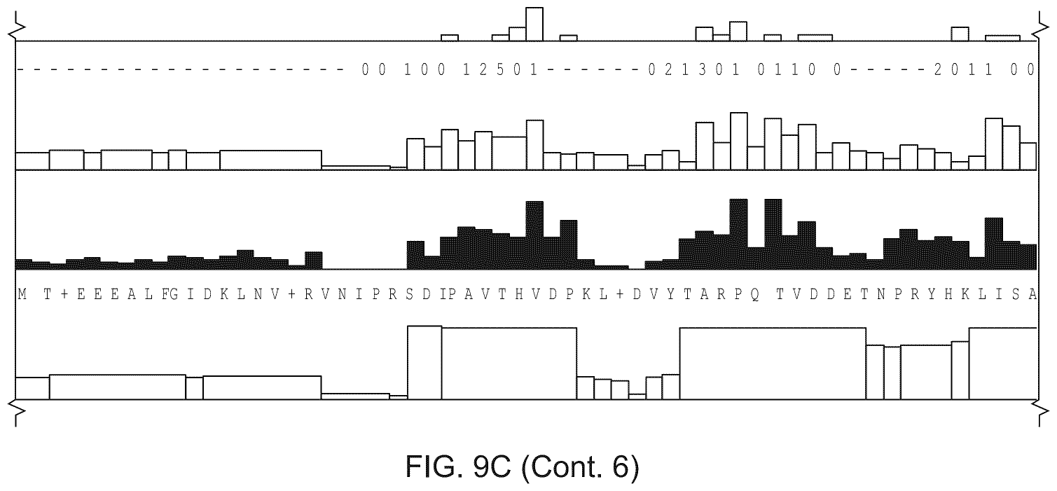

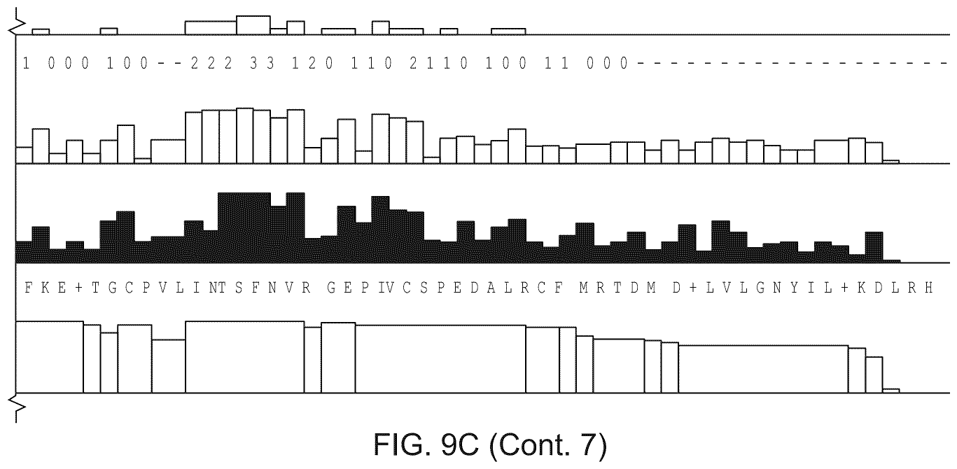

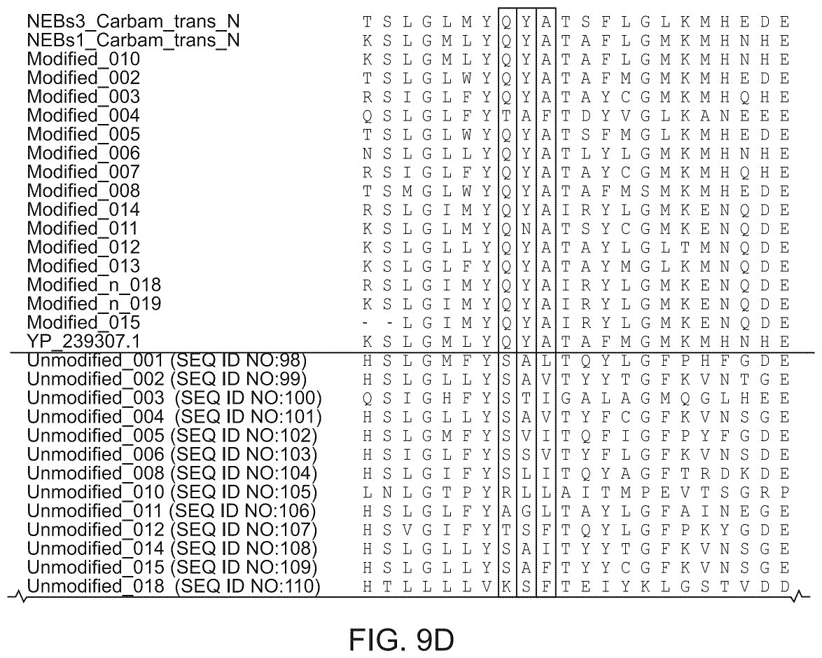

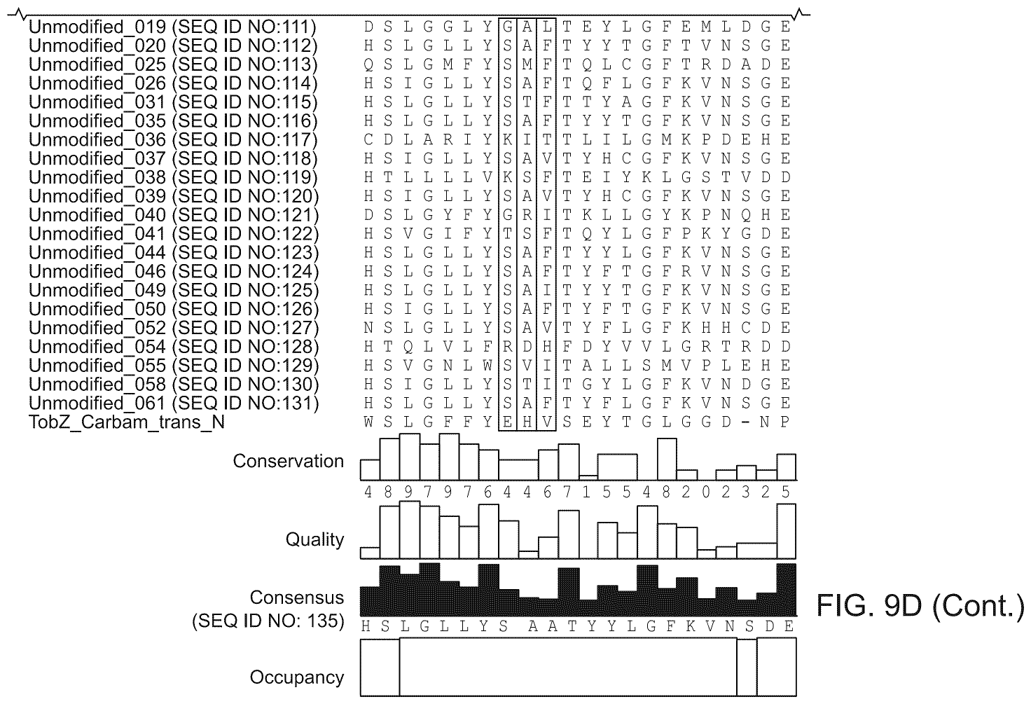

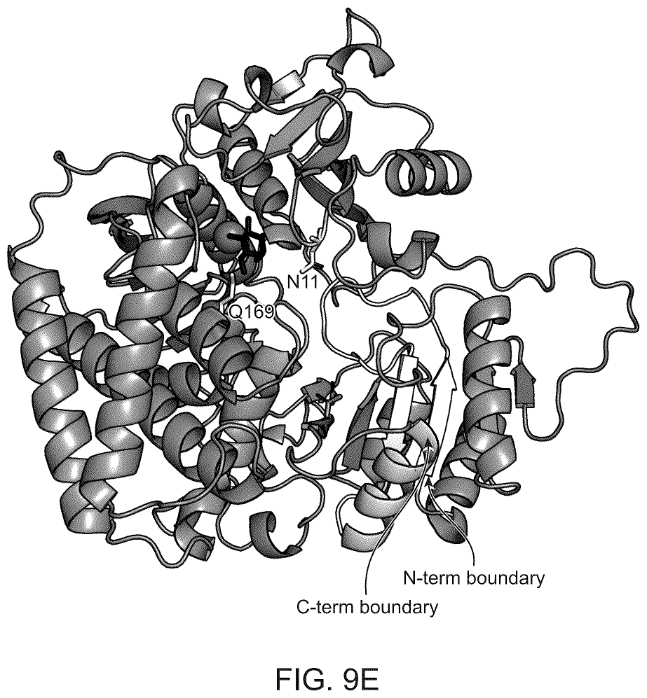

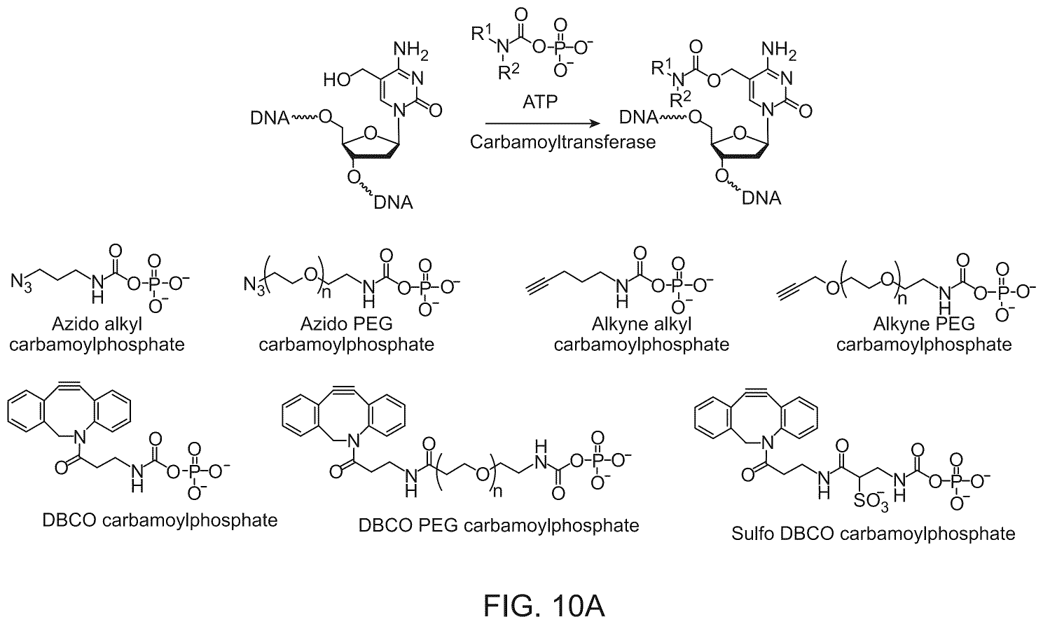

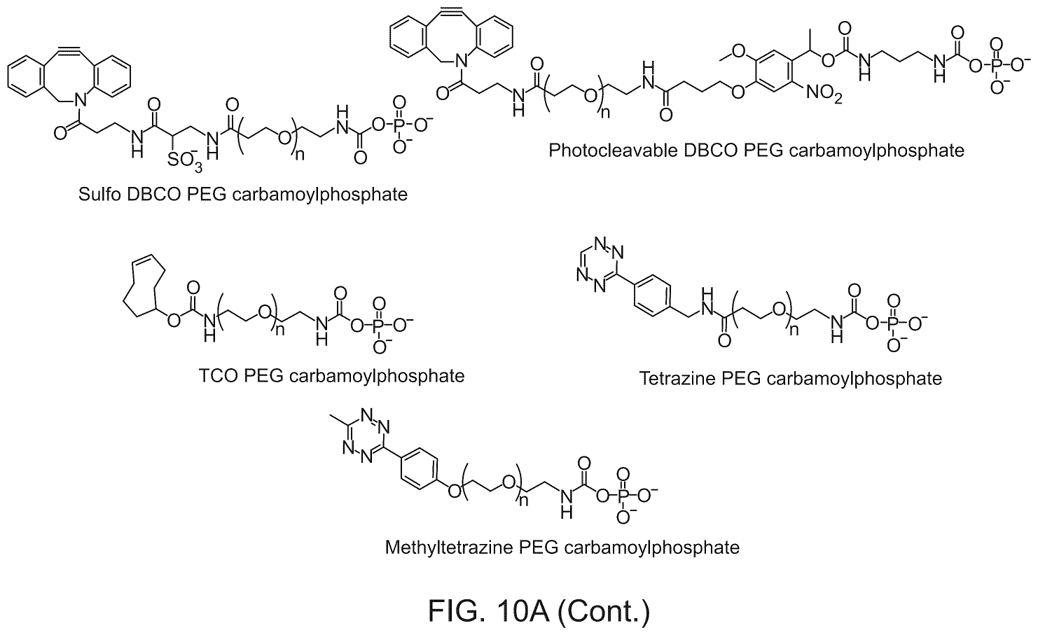

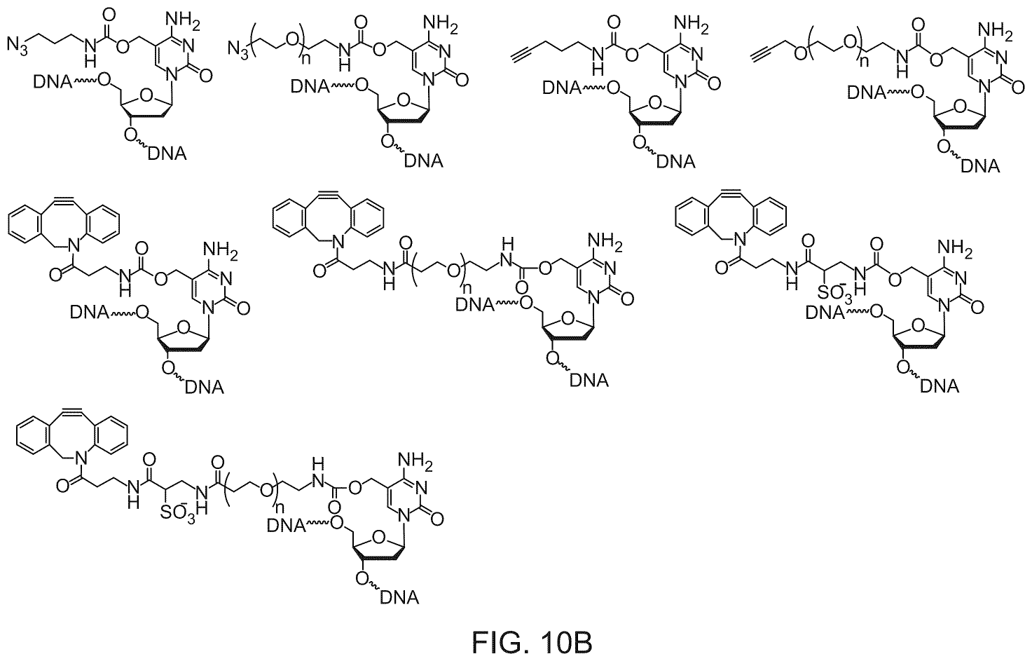

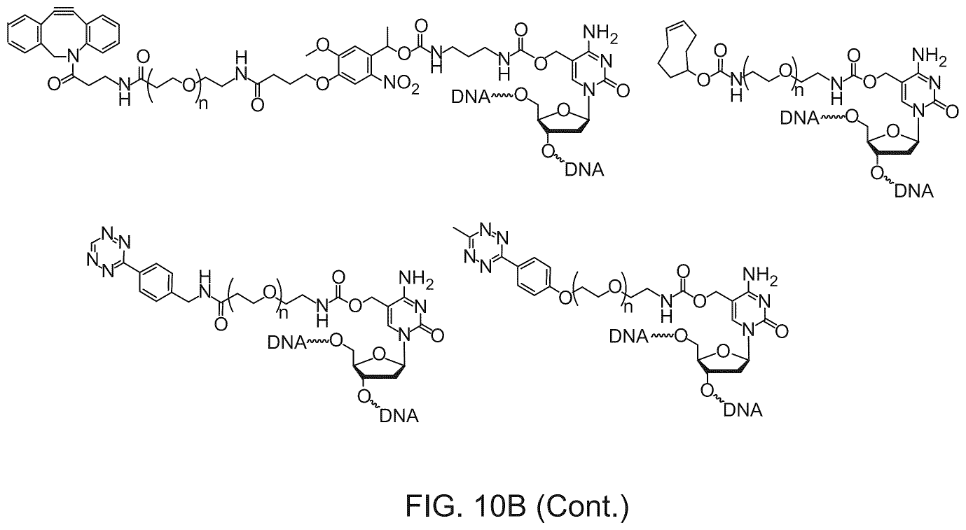

FIG. 2 provides additional explanations for the methodology described in FIG. 1.

Using Meta GPA, functional amino acid sequence units (e.g., Pfam domains) were identified that were significantly associated with DNA modifications (orange bar now black and white speckled boxes). Association analyses at single functional unit and multifunctional-unit levels were performed to discover associations with the selected phenotype (red now speckled circle). The residue differential conservation is shown in the table below.

| Domain A | Domain B |
|---|---|
| EVGPRALCNTSLL | RVKDREWWRPFA |
| SEQ ID NO: 84 | SEQ ID NO: 90 |
| EYGPRALCNTTTL | RMINNRNTVMPVA |
| SEQ ID NO: 85 | SEQ ID NO: 91 |
| EYGPRALCHTSTL | RMNDRTNEMPFE |
| SEQ ID NO: 86 | SEQ ID NO: 92 |
| EFGPRALGNRSIL | TMNNRNTVMPMA |
| SEQ ID NO: 87 | SEQ ID NO: 93 |
| EUGPRALGHRTIF | ELNDRTFVMPMAP |
| SEQ ID NO: 88 | SEQ ID NO: 94 |
| ETF-ARALGNRSVI | --ANNRNTFMPMAI |
| SEQ ID NO: 89 | SEQ ID NO: 95 |

FIG. 3A-3C describes an assay used to discover an enzyme capable of executing a targeted phenotype. In this case, the targeted phenotype is nucleotide modification in phage genomes. The presence of nucleotide modifications were detected following deamination followed by cleavage of uracils with USER® (New England Biolabs, Ipswich, MA).

FIG. 3A shows a mixture of unmodified and modified DNA to which adapters are attached. Enzyme selection is carried out and the sample divided into 2 aliquots, one aliquot being treated with USER, the other with TET/BGT and APOBEC followed by USER. The products of the reactions are then sequenced. Unknown forms of cytosine modification (denoted “x”) were recognized by blocked C-to-U deamination.

FIG. 3B shows the different sequencing outcomes for unmodified DNA (regular DNA with cytosine- GCTTAGA) and variously modified DNA with an unknown modification on cytosine (C and XC), methyl group on C (5-mC) and hydroxymethyl group on C (5-hmC) (modified DNA-^{X}CA^{m}CTG^{hm}CT). Both “modified” and “regular” samples were treated with TET and a GT for converting mC to carboxycytosine (5-^{ca}C) and 5-^{hm}c to 5-^{ghm}c. Deamination of DNA in both samples resulted in the conversion of unmodified C to Uracil (U). Regular DNA and modified DNA can be distinguished readily by treating both samples with USER that cleaves DNA at U as shown.

FIG. 3C shows the results of the sequencing. Three different DNA substrates were used to detect activity of the phage lysate. These were phage T4 containing DNA with hydroxymethylated cytosine having a deletion of the beta-glucosyltransferase (BGT) gene (T4gt), phage Xp12 containing DNA having methylated cytosine and E.coli containing a low amount of methylated cytosine and no hmC. Selection was achieved according to whether USER cleaved DNA using the total population of phage lysate. The Y-axis was labeled: “Recovery from untreated %” meaning recovery of phage nucleotide blocking activity from the total population of phage DNA.

FIG. 4A shows that using the selection of DNA modification, the highest frequency of domains in the library of phage DNAs corresponded to CT and associated enzymes in the pathway used by phage to generate protected DNA.

FIG. 4B shows the enrichment score for libraries made from selected DNA (containing modifications) and from the total library.

FIG. 4C shows the contigs obtained from the libraries of DNA containing modified DNA (modified) compared to the total libraries (unmodified) color coded for protein domains (Pfam) encoded by these contigs.

FIG. 4D shows the network occurrence relationship of the identified protein domains.

FIG. 5A shows how protein domains were found in enriched libraries that related to the pathway in which the identified CT was active. Contigs revealed that the gene encoding CT that protected modified cytosine was adjacent to a DNA region encoding thymidylate synthetase, an enzyme that is involved in reductive methylation of deoxyuridine monophosphate (dUMP) to form deoxythymidine monophosphate (dTMP).

FIG. 5B: Once the DNA encoding the Pfam contigs was identified, it was purified first on a HisTrap column and then with a Qcolumn. This DNA sequence was then cloned, expressed and characterized as a DNA modifying CT.

FIG. 6 shows the activity of the DNA modifying CT and its preferred substrate described by Formula 1.

FIG. 7A-7D shows that DNA modifying carbamoylation preferentially occurs on 5-hmC nucleotides in single stranded DNA, RNA, and in hydroxymethylated nucleoside triphosphates by hmC-CT.

FIG. 7A shows the pathway of carbamoylation by hmC-CT, where the hmC-CT catalyzes the addition of the carbamoyl group onto the pyrimidine.

FIG. 7B shows and HPLC profile for single stranded DNA in which a peak corresponding to 5-cmdC is indicated with an arrow in the sample containing hmC-CT whereas the sample without the hmC-CT shows a distinct peak corresponding to unmodified 5-hmdC.

FIG. 7C shows the HPLC profile of nucleoside triphosphate in which 5-hmdCTP is clearly distinguished from 5-cmdCTP.

FIG. 7D shows substrate specificity for the hmC-CT comparing modification of dC, 5-methyl deoxyribocytosine (5-mdC), and 5-hmdC substrates in different triplet sequences in single stranded DNA showing minimal nucleotide context bias.

FIG. 7E shows conversion percentages for comparison for 5-hmC RNA.

FIG. 7F shows conversion percentages for 5-hydroxymethylated ribocytosine triphosphate (5-hmrCTP)) with 5-hmrCTP substrate being converted at nearly 100%.

FIG. 8A shows that the hmC-CT, ATP and carbamoyl phosphate convert 5-hmdC to 5-cmdC in a single stranded DNA. Omission of one of these reagents or substitution of double stranded DNA for single strand DNA resulted in the absence of observable conversion of 5-hmdC as deduced from peak positions using HPLC.

FIG. 8B confirms that 5-hmrCTP, 5-hmdCTP and 5-hmC RNA are substrates for hmC-CT whereas 5-hydroxymethyl-2′-deoxyuridine triphosphate (5-hmdUTP) and 5-methyl-2′-deoxycytidine triphosphate (5-mdCTP) is not. From top to bottom on the graph, A=5hmrCTP+enzyme, B=5hmrCTP- enzyme, C=5mdCTP+enzyme, D=5mdCTP-enzyme, E=5hmdUTP+enzyme, E-5hmdCTP-enzyme and F=5hmdCTP+enzyme.

FIG. 8C shows that peaks for 5-cmrC and 5-hmrC are observed for 5-hmC RNA substrate under the experimental conditions used.

FIG. 9 shows the sequence properties that distinguish hmC-CTs (each sequence in the alignment labelled “modified”) from other CTs. Sequence homology at various amino acid positions are shown below the alignments. Consensus sequences are also provided below the alignments as indicated

FIG. 9A shows the sequence alignment for 17 sequenced isolates of hmC-CTs from bacteriophage and the bacterial enzyme-TobZ CT which does not have the observed hmC-CT activity.

FIG. 9B shows the results of aligning the N-terminal domain of 28 sequenced isolates.

FIG. 9C shows highly conserved amino acid residues in the c terminal domain of hmC-CT that characterize this family of enzymes. It can be seen from the alignments that the amino acids at the identified positions differ from corresponding positions in CTs that do not modify hmCs and are here labelled “unmodified”.

FIG. 9D shows highly conserved amino acid residues in the N-terminal domain of hmC-CT that characterize this family of enzymes. It can be seen from the alignments that the amino acids at the identified positions differ from corresponding positions in CTs that do not modify hmCs and are here labelled “unmodified”.

FIG. 9E shows the predicted structure of hmC-CT defined by SEQ ID NO: 1 in which the N-terminal domain amino residues are shown as being part of the catalytic domain while the C-terminal domain cluster in a different region of the protein identified by a white ribbon that includes a beta pleated sheet in the right and center of the protein structure. The C-term boundary and the N-term boundary marked on the structure refer to the boundaries of the C-terminal domain shown in FIG. 9A and also in FIG. 9C

FIG. 10A shows examples of tagged carbamoyl phosphate.

FIG. 10B shows examples of tagged cmC.

### DETAILED DESCRIPTION

Nucleotide base modifications are found in genomes and serve various purposes. For example, prokaryotes, modified bases have been described that protect the bacterial genome from its own toxic endonucleases directed toward invading bacteriophage. Bacteriophage encode enzymes that can modify their own genomes to protect against the bacterial host enzymes. Eukaryotes have adopted some of these base modifications for different purposes. For example, 5-methyl cytosine (mC) has been extensively studied in eukaryotic genomes as these modified bases regulate gene expression through transcription. Changes in the pattern of occurrences of these nucleotides in the genome can be correlated with disease.

It has not been easy to differentiate mC from hmC by eukaryotic genome sequencing and improvements in existing methods are desirable. Existing methods either use chemistry (bisulfite sequencing) that significantly damages the DNA or the addition of glucose onto hmC to prevent its oxidation to 5-carboxycytosine (CaC) by the eukaryotic methylcytosine dioxygenase-TET. A significant improvement over bisulfite sequencing has been the additional use of a deaminase that acts on single stranded nucleic acids to convert cytosine and unmodified mC to uracil and thymine respectively (see for example U.S. Pat. Nos. 10,619,200 and 10,260,088). Alternatively, labelled glucose has been transferred onto hmC for direct detection of this modified nucleotide (see for example US 2014/0322707).

An improvement over existing methods would be to find alternatively molecules that can bind to hmC in single strand DNA that could be combined with deaminase in a single reaction to simplify and improve workflow design. Here a new family of enzymes were identified that achieve this desired step. In addition to the above uses, this new family of enzymes have additional advantages in other methods that include methods for stabilization, detection, enrichment and/or sequencing of polynucleotides as outlined below.

The initial step of discovery was to recognize that bacteriophage were likely to encode the enzyme or enzymes responsible for any base modifications that might occur to protect its own genome from toxic bacterial host enzymes. The next step was to search an environment that was sufficiently diverse with respect to phage to provide the opportunity to discover such enzymes and base modifications and to develop an assay that would enable detection of phage nucleic acids that contained modified cytosine that were resistant to deaminase and thereby to detect coding sequences in the nucleic acids for enzymes that could catalyze such modifications. The assay used for initial screening is described in FIG. 3B as part of a detailed description of the methods in FIG. 1, FIG. 2 and FIGS. 3A and 3B.

To discover novel base modifications developed by bacteriophage to overcome bacterial immune systems for use in these methods, a metagenome analysis (Meta GPA) of environmental samples was undertaken. Bacteriophage have proved particularly adept in utilizing base modifications to protect their nucleic acid from destruction by host bacteria. Examples of base modifications include 5-(2-aminoethoxy)methyluridine, 5-(2-aminoethyl)uridine and 7-deazaguanine (Lee,. et al. Proc. Natl. Acad. Sci. U. S. A. 115, E3116-E3125 (2018); Hutinet, et al. Nat. Commun. 10, 5442 (2019)). To achieve such base modifications, bacteriophage genomes encode enzymes that catalyze nucleotide modification reactions of their own genomes.

A Meta GPA workflow (see for example FIGS. 1, 2, 3A-B, 4A-4D) was successfully implemented using environmental DNA. The workflow included linking functional phenotype with genetic information. A family of hmC-CT was surprisingly identified that reacted with carbamoyl phosphate to add a carbamoyl group onto hmC in DNA and RNA preferring single stranded nucleic acids and also hmdCTP and hmrCTP triphosphates to form cmC, The term “CmC” is intended to cover modified nucleoside triphosphates as well as modified bases in a nucleic acid (see for example, FIGS. 5A-5B, 6, 7A-F, 8A-8C, 9A-9D, and 10A-10B). This novel enzyme family is here referred to as hmC-CT. The substrate of hmC-CT is carbamoyl phosphate or derivatives thereof.

The abbreviations of mC, hmC, cmC, hmdC, hmrC, hmdCTP and hmrCTP are used interchangeably with 5-mC, 5-hmC, 5-cmC, 5-hmdC, 5-hmrC, 5-hmdCTP and 5-hmrCTP where the “5” refers to the position on the pyrimidine (in this case, cytosine). However the abbreviations refer to molecules that are not limited to modifications at the “5” position as indicated in the figures but may include other positions on the pyrimidine.

The method used to identify this family of 5-hmC-CT was as follows: Intact phage particles were rescued from microbiomes from sewage or coastal environments. These virus particles were lysed to form a library of total phage DNA. Aliquots of the library of total phage DNA were screened enzymatically in an assay that utilized a deaminase and a nicking agent (USER). The assay involved degradation by USER of “regular” DNA that had unprotected cytosine (see FIG. 3A). This degradation was observed when cytosine was deaminated by APOBEC to form uracil that was subsequently degraded by uracil deglycosylase (UDG). Modified DNA, in which all cytosine was converted to 5-hmC that was protected by a chemical group such as glucose, was not degraded by USER.

When the modified DNA was analyzed and contigs formed, it was found using Pfam analysis of the contigs that various protein domains could be identified using single and multidomain analysis. These protein domains were found to correspond to a carbamoyltransferase (referred to herein and in the figures as hmC-CT or “modified”) that was observed to frequently co-occur with thymidylate synthetase. Thymidylate synthase (TS) homologues can add methyl or hydroxymethyl groups to the pyrimidine ring of a deoxynucleotide monophosphate. The hydroxymethyl groups can serve as sites for further modification (hypermodification) after DNA replication.

When the substrate specificity of the DNA modifying activity of hmC-CT was further explored, it was found that the enzyme favored single stranded DNA and RNA over double stranded DNA for modifying hmC. It was also found that the enzyme required carbamoyl phosphate where the phosphate acted as a leaving group for attaching the carbamoyl group onto the methylated cytosine. Moreover, it was found that relatively little bias occurred in the context of the modified cytosine (see for example, FIG. 7D) for carbamoylation.

The ability of these CTs to carbamoylate hmC had never been described before. Subsequent sequence analysis revealed that these enzymes belonged to a distinct and separate family of enzymes which certain common characteristics. This family is here described as hmC-CT. Certain features of this family differentiate them from CTs that do not have the hmC modification activity.

Distinguishing features of hmC-CT included one or more of the following characteristics:
(a) Transfer of a carbamoyl group onto a hmC that is a deoxyribonucleoside triphosphate, a ribonucleoside triphosphate, or is positioned in a double stranded or single stranded nucleic acid sequence where the nucleic acid is DNA or RNA;
(b) Relatively low sequence bias regarding the sequence context of the hmC;
(c) For wildtype hmC-CT, proximity of the gene encoding this enzyme to a thymidylate synthase gene on the viral genome; for example within 2 kb of the hmC-CT gene;
(d) Characteristic conserved amino acids;
(e) At least 80% sequence identity to an amino acid sequence in the C-terminal domain corresponding to position 393- 434 of SEQ ID NO: 1 and optionally in the N-terminal domain, a glutamine (Q) at a position corresponding to 169 and an alanine (A) at a position corresponding to 171 in SEQ ID NO: 1;
(f) a glutamine (Q) at a position corresponding to 169 and an alanine (A) at a position corresponding to 171 in SEQ ID NO: 1 and optionally at least 80% sequence identity to an amino acid sequence in the C-terminal domain corresponding to position 390-435 of SEQ ID NO: 1; and/or
(g) A preference for modifying hmC in single stranded nucleic acids over double stranded nucleic acids.

Several examples of naturally occurring amino acid sequences for the family of hmC-CT enzymes are provided in FIGS. 9A and 9B. This set is not intended to be limiting but is merely representative of the library derived from the sewage that was sampled. It will be apparent to a person of ordinary skill in the art, the methods utilized herein may be applied to microbiomes from any environmental sample, so as to form DNA libraries and select and clone CTs for the uses described herein.

Consensus amino acid sequences for 5hmC -CT may include:

In the C-terminal domain:
(a)
(SEQ ID NO: 3)
LINTSFNYHGVPIVLD+EQIIH+HFM
In the N-terminal domain:
(b)
(SEQ ID NO: 4)
DRVIIAYYVQRVLESVVLKL+K.
(c)
(SEQ ID NO: 5)
SDLYKPKNLILSGGVFYNVKLNN+ILDK.
(d)
(SEQ ID NO: 6)
MPLAGDQGAALGA

The identified 5-hmC may vary in the region of the consensus sequence but nonetheless retain at least 50%, at least 60%, at least 70%, at least 80%, or at least 90% sequence identity to one or more of these N-terminal and/or C-terminal sequences (SEQ ID NOs: 3-6).

In one embodiment, an hmC-CT is generally at least 80% or 90% identical (e.g., at least 91% , 92%, 93%, 94%, 95%, 96%, 97%, 98% or 99% identical) to SEQ ID NO: 1, 29-47, 49 and 96-97.

In FIG. 9A-9D, conserved amino acids in the C-terminal domain are provided below the sequence comparisons. Accordingly, preferably hmC-CT has the following conserved amino acids:
the position corresponding to position 393 in SEQ ID NO:1 is generally an asparagine (N), not a glycine(G) nor an alanine (A);
the position corresponding to position 394 in SEQ ID NO:1 is generally an isoleucine (I), leucine (L), valine (V) or phenylalanine (F), not a tryptophan (W) or histidine (H); and if the amino acid is a V then it occurs as a triplet of NVV at position 394-396, and if it is an L then it is occurs in a triplet of NLV at position 394-396;
the position corresponding to position 395 in SEQ ID NO: 1 is a V or an F and if it is an F than there is an His position 396, a G in position 397 and an Aspartic acid (D) in position 398;
the position corresponding to position 398 in SEQ ID NO: 1 is generally an N, serine (S), lysine (K) or D but not an arginine (R) nor an A;
the position corresponding to position 407 in SEQ ID NO: 1 is generally a cysteine (C), or a glycine (G) but if it is a G then there is a threonine (T) at positions corresponding to 409 and 411;
the position corresponding to position 409 in SEQ ID NO: 1 is generally a T and not an R;
the position corresponding to position 411 in SEQ ID NO: 1 (position 425 in TobZ) is generally a T or C, not an I or F;
the position corresponding to position 416 in SEQ ID NO: 1 is generally a D or a proline (P) but when it is a D it is adjacent to a D at position 417;
the position corresponding to position 428 in SEQ ID NO: 1 is generally an N and not a K;
the position corresponding to position 434 in SEQ ID NO: 1 is generally an methionine (M) and not an R where the M is proximate to an N at position 428;
and
the position corresponding to position 460 in SEQ ID NO: 1 (477 in Tob Z) is generally not a proline (P), S, K or Y.

Examples of conserved amino acid residues in the N-terminal domain are highlighted in FIG. 9D as follows:
the position corresponding to position 169 in SEQ ID NO: 1 is generally a glutamine (Q); the position corresponding to position 170 in SEQ ID NO:1 is generally a tyrosine (Y), alanine (A) or asparagine (N);
the position corresponding to position 171 in SEQ ID NO:1 is generally an A.

In some embodiments, the hmC-CT may have amino acids specified in 1, 2, 3, 4, 5, 6, 7, 8, 9, 10, 11, 12, 13, 14, or 15 positions described above. These amino acids may also be suitable for targeted mutations to modify or improve the activities of these enzymes.

FIG. 9E describes the predicted structure of SEQ ID NO: 1. This hmC-CT was deduced using alpha fold Al predictions. The N-terminal domain conserved amino acids at 169-171 are positioned in the putative active site of the enzyme while the C-terminal domain containing the 12 conserved amino acids described above is shown by a white ribbon bordered by black lines. In certain embodiments, mutations to the amino acids in the N-terminal domain around the active site or the c-terminal domain that may contribute to the surface properties of the enzyme may be suitable targets for mutation to improve desired properties of these enzymes.

The conserved amino acids are presumed to affect the structure of the family of hmC-CTs to differentiate them from “unmodified” CTs described in SEQ ID NOs 50-83 corresponding to C-terminal domains and N-terminal domains of non-hmC CTs isolated from the same metagenome as the hmC-CT sequences.

CTs have been described in prokaryotes and mammals with varied but substantially different functions. For example, prokaryotic CTs catalyzed the reaction between carbamoyl phosphate (CP) and ornithine (Orn) to form citrulline (Cit) and phosphate (Pi) in the biosynthesis pathway of arginine (see for example, Tuchman et al (2002) Human Mutation, 19 (2): 93-107). Tob Z is an example of an O-carbamoyltransferase in bacteria that adds a carbamoyl group onto the antibiotic tobramycin to form nebramycin. Mammalian CT was also identified in mammals where it was reported to play a significant role in the urea cycle or as a first step in pyrimidine biosynthesis, where I-aspartate and carbamoyl phosphate condense to form N-carbamoyl-L-aspartate and inorganic phosphate.

While not wishing to be limited by theory, it is possible that bacteriophage co-opted a prokaryotic enzyme, namely CT for a different purpose. Instead of pyrimidine biosynthesis, the bacteriophage may have adapted the same enzyme for modification of hmC, hmrCTP and hmdCTP to protects its DNA from cleavage in an infected host bacterial cell. It may be expected therefore that the multiple sequence variants of the hmC-CT found to be encoded in the bacteriophage DNA resulted from the acquisition of this enzyme relatively recently in evolutionary time. Consequently, hmC-CT including derivatives or mutants thereof, found in viruses, would be expected to be interchangeable with the hmC-CT used in the examples below.

Owing to the natural variation of the hmC-CT obtained via Meta GPA analysis described here, it is probable that further variants will be found in the bacterial virus population from other metagenomic libraries. Moreover, it would be expected that this degree of variation could be mimicked in the laboratory without necessarily altering the novel phenotypic properties of this enzyme. However, it is expected that the hmC-CT may be mutated in vitro or in vivo to improve features such as enzyme substrate specificity and/or enzyme kinetics and/or ease of manufacture and/or stability at various temperatures and in various buffers.

The hmC-CT may be modified in vitro by for example fusing part or all of the protein to a protein domain from a non-viral source (for example, fusion to maltose binding protein (MBP); for binding to an affinity substrate, for example, chitin binding domain or MBP etc.). Where the protein is complex with multiple domains, for example a trimer, then individual protein domains may be fused to each other or to non-viral protein domains to facilitate production and purification of the hmC-CT in vitro.

The substrate of hmC-CT is a carbamoyl group, for example, carbamoyl phosphate or tagged carbamoyl phosphate. Carbamoyl phosphate is relatively stable since the carbonyl group is stabilized by the amine. The phosphate acts as a leaving group by reacting with the target of the transferase that receives the carbonyl group releasing the phosphate group.

* As used herein, the term “carbamoyl phosphate substrate” is used to refer to both an “untagged” carbamoyl phosphate shown in Formula 1 and a “tagged” carbamoyl phosphate in which a chemical group is added to R1 or R2 as described below that may comprise in addition to a chemically reactive group, a functional group and./or a linker.

### Substrates for Hydroxymethylcytosine-Carbamoyltransferase

Formula 1 below (also see FIG. 6 and FIG. 10A) is characterized by a carbonyl group and NR₁R₂. The phosphate is a transfer group allowing the O═C—N—R₁R₂ to become attached to the Oxygen of the hydroxyl group on the methyl cytosine. The R1 and R2 groups permit the hmC to be tagged with a chemical reactive group; and optionally a functional group such as a spectroscopic probe, a radioactive probe, an affinity moiety, and a nucleic acid; and/or a linker.

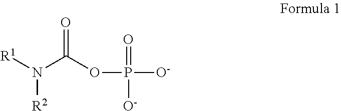

The R1 and R2 in Formula 1 independently of each other may be an H or a tag (T) comprising a chemically reactive group (C) a functional group (F) and/or a linking group (L) where the linking group may be positioned between the carbamoyl group and the chemically reactive group and /or between the chemically reactive group and the functional group.

### The Chemically Reactive Group

Examples of suitable chemically reactive groups at R1 or R2 include a carbonyl; a carboxyl; an active ester, e.g., a succinimidyl ester; a maleimide; an amine; a thiol; an alkyne, an azide; an alkyl halide; an isocyanate; an isothiocyanate; an iodoacetamide; a 2-thiopyridine; a 3-arylproprionitrile; a diazonium salt; an alkoxyamine; a hydrazine; a hydrazide; a phosphine; an alkene; a semicarbazone; an epoxy; a phosphonate; and a tetrazine, for example one of a succinimidyl ester, a maleimide, an amine, a thiol, an alkyne, or an azide. Other examples include a chemical moiety that is capable of (i) crosslinking to other molecules (e.g. benzophenone), (ii) generating hydroxyl radicals upon exposure to H202 and ascorbate (e.g. a tethered metal-chelate), (iii) generating reactive radicals upon irradiation with light (e.g. malachite green), or a molecule possessing a combination of any of the properties listed above.

Examples of chemical reactions with the above reactive groups include reactions between an amine reactive group and an electrophile such an alkyl halide or an N-hydroxysuccinimide ester (NHS ester); between a thiol reactive group and an iodoacetamide or a maleimide; between an azide and an alkyne (azide-alkyne cycloaddition or “Click Chemistry”).

Examples and uses of such chemically reactive groups in biological systems are reviewed in a variety of publications, such as in Sletten, E. M. and Bertozzi C. R. “Bioorthogonal Chemistry: Fishing for Selectivity in a Sea of Functionality” Angewandte Chemie International Edition English 2009, 48(38): 6974-98. When R¹ or R² is an azido or alkyne, a Cu(I)-catalyzed or strain promoted 1,3-dipolar cycloaddition between azide and the alkyne derivative yields the 1,4-substituted triazole. A

lternatively, the azide and a cyano derivative react under Lewis acid catalysis (ZnBr₂) to form tetrazole. A variety of different chemoselective groups may be used. For example, bis-NHS esters and maleimides (which react with amines and thiols, respectively), may be used. In other cases, the chemoselective group on the nucleoside may react with a reactive site on suitable reagent or substrate via click chemistry. In these embodiments, the nucleoside may contain an alkyne or azide group. Click chemistry, including azide-alkyne cycloaddition, is reviewed in a variety of publications including Kolb, et al., Angewandte Chemie International Edition 40: 2004-2021 (2001), Evans, Australian Journal of Chemistry, 60: 384-395 (2007) and Tornoe, Journal of Organic Chemistry, 67: 3057-3064 (2002).

### Functional Groups

In some embodiments, the tag T in R1 or R2 may include a functional group such as a detectable label such as fluorophore, a chromophore, a magnetic label, a contrast reagent, a radioactive label or the like, where these detectable labels may generate signals that can be detected by standard means and may be used in vitro or in vivo. Exemplary detectable labels include optically detectable labels (e.g., fluorescent, chemiluminescent or colorimetric labels), radioactive labels, and spectroscopic labels such as a mass tag. Exemplary optically detectable labels include fluorescent labels such as xanthene dyes, e.g. fluorescein and rhodamine dyes, such as fluorescein isothiocyanate (FITC), 6 carboxyfluorescein (commonly known by the abbreviations FAM and F),6 carboxy-2′,4′,7′,4,7-hexachlorofluorescein (HEX), 6 carboxy 4′, 5′ dichloro 2′, 7′ dimethoxyfluorescein (JOE or J), N,N,N′,N′ tetramethyl 6 carboxyrhodamine (TAMRA or T), 6 carboxy X rhodamine (ROX or R), 5 carboxyrhodamine 6G (R6G5 or G5), 6 carboxyrhodamine 6G (R6G6 or G6), and rhodamine 110; cyanine dyes, e.g. Cy3, Cy5 and Cy7 dyes; coumarins, e.g. umbelliferone; benzimide dyes, e.g. Hoechst 33258; phenanthridine dyes, e.g. Texas Red; ethidium dyes; acridine dyes; carbazole dyes; phenoxazine dyes; porphyrin dyes; polymethine dyes, e.g. cyanine dyes such as Cy3, Cy5, etc .; BODIPY dyes and quinoline dyes. Specific fluorophores of interest that are commonly used in some applications include: pyrene, coumarin, diethylaminocoumarin, FAM, fluorescein chlorotriazinyl, R110, eosin, JOE, R6G, tetramethylrhodamine, TAMRA, lissamine, ROX, napthofluorescein, Texas red, napthofluorescein, Cy3, Cy5, and FRET labels, etc. The label can be detected directly or indirectly. Indirect detection means that the label is detected after interaction or reaction with another substrate or reagent. For example, through chemical conjugation, affinity partner binding, epitope binding with an antibody, substrate cleavage by an enzyme, donor-acceptor energy transmission (e.g., FRET), etc. Label combinations for tandem affinity purification found in the literature was summarized in Li, Biotechnol. Appl. Biochem, 55:73-83 (2010).

In some embodiments, the tag T in R1 or R2 may include a functional group such as an affinity label moiety. In such embodiments, the affinity tag may be used to enrich for DNA comprising the affinity tag-labeled carbamoyl cytidine using an affinity matrix that binds to the affinity tag. In any embodiment, this method may further comprise chemically cleaving a cleavable linker between the affinity moiety and the carbamoyl cytidine, thereby releasing the enriched DNA from the affinity matrix. Affinity labels are moieties that can be used to separate a molecule to which the affinity label is attached from other molecules that do not contain the affinity label. In many cases, an affinity label is a member of a specific binding pair, i.e., two molecules where one of the molecules through chemical or physical means specifically binds to the other molecule. The complementary member of the specific binding pair, which can be referred to herein as a “capture agent” may be immobilized (e.g., to a chromatography support, a bead or a planar surface) to produce an affinity chromatography support that specifically binds the affinity tag. In other words, an “affinity label” may bind to a “capture agent”, where the affinity label specifically binds to the capture agent, thereby facilitating the separation of the molecule to which the affinity tag is attached from other molecules that do not contain the affinity label. Exemplary affinity tags include, but are not limited to, a biotin moiety (where the term “biotin moiety” is intended to refer to biotin and biotin analogs such as desthiobiotin, oxybiotin, 2′-iminobiotin, diaminobiotin, biotin sulfoxide, biocytin, etc., that are able to bind to streptavidin with an affinity of at least 10-8 M), avidin, streptavidin, protein A, maltose-binding protein, chitin binding domain, SNAP-tag poly-histidine, HA-tag, c-myc tag, FLAG-tag, GST, an epitope binding molecule such as an antibody, and polynucleotides that are capable of hybridizing to a substrate but excludes an alkyl group.

Moieties combinations for tandem affinity purification found in the literature was summarized in Li, Biotechnol. Appl. Biochem, 55:73-83 (2010). The table on page 74 of Li included the following where affinity tag/sequence or size (KDa)/Affinity matrix/Elution strategy is presented:

TABLE 2

| Affinity | | Affinity | Elution |
|---|---|---|---|
| tag | Sequence or size (Kda) | matrix | strategy |
| Z domain* | VDNKFNKEQQNAFYEILHLPNLNEEQRNAFIQSLK | IgG | IgG or low pH |
| | DDPSQSANLLAEAKKLNDAQAPK (SEQ ID NO: 7) | | |
| CBP | KRRWKKNFIAVSAANRFKKISSSGAL (SEQ ID | Calmodulin | 2 mM EGTA |
| | NO: 8) | | |
| His-tag | HHHHHH (SEQ ID NO: 9) | Ni2+, Co2+ | 150-500 mM imidazole |
| FLAG | DYKDDDDK (SEQ ID NO: 10) | Antibody | FLAG peptide or low pH |
| HA | YPYDVPDYA (SEQ ID NO: 11) | Antibody | HA peptide or low pH |
| Myc | EQKLISEEDL (SEQ ID NO: 12) | Antibody | Low pH |
| V5 | GKPIPNPLLGLDST (SEQ ID NO: 13) | Antibody | V5 peptide or low pH |
| Strep II | WSHPQFEK (SEQ ID NO: 14) | StrepTactin | 2.5-5 mM desthiobiotin |
| SBP | MDEKTTGWRGGHVVEGLAGELEQLRARLEH | Streptavidin | 2 mM biotin |
| | HPQGQREP (SEQ ID NO: 15) | | |
| S-peptide | KETAAAKFERQHMDS (SEQ ID NO: 16) | S-protein | Denaturant or low pH |
| CBD | TNPGVSAWQVNTAYTAGQLVTYNGKTYKCLQPH | Chitin | Thiol reagents or pH and |
| | TSLAGWEPSNVPALWQLQ (SEQ ID NO: 17) | | temperature shift (when |
| | | | fused with intein) |
| GST | 26 | Glutathione | 10 mM reduced |
| | | | glutathione |
| MBP | 40 | Maltose | 10 mM maltose |

*Z domain is a synthetic Fc-region-binding domain derived from the B domain of ProtA.

An advantageous feature of a desthiobiotin label is that it binds streptavidin less tightly than biotin and can be displaced by biotin ensuring that elution of enriched DNA is readily achieved.

In some embodiments, the tag T in R1 or R2 may include a functional group that is an oligoribonucleotide or an oligodeoxyribonucleotide, attached to the linker in either a 5′ to 3′ or a 3′ to 5′ orientation, a peptide nucleic acid (PNA), a lock nucleic acid (LNA), an unlock nucleic acid (UNA), a triazole nucleic acid, or a combination thereof.

In some embodiments, the tag T in R1 or R2 may be include a functional group such a lipid or other hydrophobic molecule with membrane-inserting properties, a benzylguanine, a benzylcytosine, a saccharide, an OH group, a cyano group, a trifluoromethyl group, a nitro group, a lower alkyl group (e.g. methyl, ethyl), a lower alkoxy group (e.g. methoxy), a lower acyloxy group (e.g. acetoxy), a lower acylamine group (e.g. acetamide), an aryl group (e.g. phenyl, benzyl), a cycloalkyl group, or an heterocyclylyl group (e.g., triazolyl).

In some embodiments, the tag T in R1 or R2 permit any variety of subsequent analysis of the labeled DNAs, including and without limitation isolation, purification, immobilization, identification, localization, amplification, and other such procedures known in the art.

### Linker Group

In some embodiments, the tag T in R1 or R2 may be separated from the carbamoyl core by a linker L. The linker L may be a flexible and may serve as steric spacers but do not necessarily have to be of defined length. Examples of suitable linkers may be selected from any of the hetero-bifunctional cross linking molecules described by Hermanson, Bioconjugate Techniques, 2nd Ed; Academic Press: London, Bioconjugate Reagents, pp 276-335 (2008), incorporated by reference.

The linker L can also increase the solubility of the compound in the appropriate solvent. The linkers used are chemically stable under the conditions of the actual application. The linker does not interfere with CT reaction nor with the detection of the labels but may be constructed such as to be cleaved at some point in time after the transferase reaction. The linker L may be a straight or branched chain alkylene group with 1 to 300 carbon atoms, wherein optionally:
(a) one or more carbon atoms are replaced by oxygen, in particular wherein every third carbon atom is replaced by oxygen, e.g., a polyethyleneoxy group with 1 to 100 ethyleneoxy units;
(b) one or more carbon atoms are replaced by nitrogen carrying a hydrogen atom, and the adjacent carbon atoms are substituted by oxo, representing an amide function —NH—CO—;
(c) one or more carbon atoms are replaced by oxygen, and the adjacent carbon atoms are substituted by oxo, representing an ester function —O—CO—;
(d) the bond between two adjacent carbon atoms is a double or a triple bond, representing a function —CH═CH— or —C═C—;
(e) one or more carbon atoms are replaced by a phenylene, a saturated or unsaturated cycloalkylene, a saturated or unsaturated bicycloalkylene, a divalent heteroaromatic or a divalent saturated or unsaturated heterocyclyl group;
(f) two adjacent carbon atoms are replaced by a disulfide linkage —S—S—; or a combination of two or more, especially two or three, alkylene and/or modified alkylene groups as defined under (a) to (f) hereinbefore, optionally containing substituents.

A linker L may be a straight chain alkylene group with 1 to 25 carbon atoms or a straight chain polyethylene glycol group with 4 to 100 ethyleneoxy units, optionally attached to a —CH═CH— or —C≡C— group. Further preferred is a straight chain alkylene group with 1 to 25 carbon atoms wherein carbon atoms are optionally replaced by an amide function —NH—CO—, and optionally carrying a photocleavable subunit, e.g., o-nitrophenyl. Further preferred are branched linkers comprising a polyethylene glycol group of 3 to 6 ethylene glycol units and alkylene groups wherein carbon atoms are replaced by amide bonds, and further carrying substituted amino and hydroxy functions. Other preferred branched linkers have dendritic (tree-like) structures wherein amine, carboxamide and/or ether functions replace carbon atoms of an alkylene group.

In one embodiment, any functionalized polyethylene glycol derivative may be used as a linker such as any of the pegylation products described in catalogs of Nanocs, Inc., Fisher Scientific, or VWR, Sigma-Aldrich Chemical, all of which are incorporated herein by reference.

A linker L may be a straight chain alkylene group of 2 to 40 carbon atoms optionally substituted by oxo wherein one or two carbon atoms are replaced by nitrogen and 0 to 12 carbon atoms are replaced by oxygen. For example, the linker R is a straight chain alkylene group of 2 to 10 carbon atoms wherein one or two carbon atoms are replaced by nitrogen and one or two adjacent carbon atom are substituted by oxo, for example a linker —CH₂—NH(C═O)— or —CH₂—NH(C═O)—(CH₂)5—NH—.

Substituents considered are e.g., lower alkyl, e.g., methyl, lower alkoxy, e.g., methoxy, lower acyloxy, e.g., acetoxy, or halogenyl, e.g., chloro.

Further substituents considered are e.g., those obtained when an a-amino acid, in particular a naturally occurring α-amino acid, is incorporated in the linker wherein carbon atoms are replaced by amide functions —NH—CO— as defined in (b) above. In such a linker, part of the carbon chain of the alkylene group is replaced by a group —(NH—CHX—CO)n— wherein n is between 1 and 100 and X represents a varying residue of an a-amino acid.

A further substituent is one which leads to a photocleavable linker, e.g., an o-nitrophenyl group. In particular this substituent o-nitrophenyl is located at a carbon atom adjacent to an amide bond, e.g., in a group —NH—CO—CH₂—CH(o-nitrophenyl)—NH—CO—, or as a substituent in a polyethylene glycol chain, e.g., in a group —O—CH₂—CH(o-nitro-phenyl)—O—. Other photocleavable linkers considered are e.g., diazobenzene, phenacyl, alkoxybenzoin, benzylthioether and pivaloyl glycol derivatives.

A phenylene group replacing carbon atoms as defined under (e) above is e.g., 1,2-, 1,3-, or preferably 1,4-phenylene. In a particular embodiment, the phenylene group is further substituted by a nitro group, and, combined with other replacements as mentioned above under (a), (b), (c), (d), and (f), represents a photocleavable group, and is e.g. 4-nitro-1,3-phenylene, such as in —CO—NH—CH₂—(4-nitro-) 1,3-phenylene-CH(CH₃)—O—CO—, or 2-methoxy-5-nitro-1,4-phenylene, such as in —CH₂—O—(2-methoxy-5-nitro-)1,4-phenylene CH(CH₃)—O—, or 2-nitro-1,4-phenylene, such as in —CO—O—CH₂—(2-nitro-)1,4-phenylene —CO—NH—. Other particular embodiments representing photocleavable linkers are e.g. - 1,4-phenylene-CO—CH₂—O—CO—CH₂—(a phenacyl group), -1,4-phenylene-CH(OR)—CO—1,4- phenylene- (an alkoxybenzoin), or -3,5-dimethoxy-1,4-phenylene—CH₂—O— (a dimethoxybenzyl moiety). A saturated or unsaturated cycloalkylene group replacing carbon atoms as defined under (e) hereinbefore may be derived from cycloalkyl with 3 to 7 carbon atoms, preferably from cyclopentyl or cyclohexyl, and is e.g., 1,2- or 1,3-cyclopentylene, 1,2-, 1,3-, or preferably 1,4-cyclohexylene, or also 1,4-cyclohexylene being unsaturated e.g., in 1- or in 2-position.

A saturated or unsaturated bicycloalkylene group replacing carbon atoms as defined under (e) hereinbefore is derived from bicycloalkyl with 7 or 8 carbon atoms, and is e.g., bicycle [2.2.1] heptylene or bicyclo [2.2.2]octylene, preferably 1,4-bicyclo[2.2.1]-heptylene optionally unsaturated in 2-position or doubly unsaturated in 2- and 5-position, and 1,4-bicyclo[2.2.2]octylene optionally unsaturated in 2-position or doubly unsaturated in 2- and 5-position.

A divalent heteroaromatic group replacing carbon atoms as defined under (e) hereinbefore may, for example, include 1,2,3-triazole moiety, preferably 1,4-divalent 1,2,3-triazole. A divalent heteroaromatic group replacing carbon atoms as defined under (e) hereinbefore is e.g., triazolidene, preferably 1,4-triazolidene, or isoxazolidene, preferably 3,5-isoxazolidene. A divalent saturated or unsaturated heterocyclyl group replacing carbon atoms as defined under (e) hereinbefore is e.g. derived from an unsaturated heterocyclyl group, e.g. isoxazolidinene, preferably 3,5-isoxazolidinene, or a fully saturated heterocyclyl group with 3 to 12 atoms, 1 to 3 of which are heteroatoms selected from nitrogen, oxygen and sulfur, e.g. pyrrolidinediyl, piperidinediyl, tetrahydrofuranediyl, dioxanediyl, morpholinediyl or tetrahydrothiophenediyl, preferably 2,5-tetrahydrofuranediyl or 2,5-dioxanediyl. A particular heterocyclyl group considered is a saccharide moiety, e.g., an α- or B-furanosyl or α- or β-pyranosyl moiety.

The extension “-ylene” as opposed to “-yl” in for example “alkylene” as opposed to “alkyl” indicates that said for example “alkylene” is a divalent moiety connecting two moieties via two covalent bonds as opposed to being a monovalent group connected to one moiety via one covalent single bond in said for example “alkyl”. The term “alkylene” therefore refers to a straight chain or branched, saturated or unsaturated hydrocarbon moiety; the term “heteroalkylene” as used herein refers to a straight chain or branched, saturated or unsaturated hydrocarbon moiety in which at least one carbon is replaced by a heteroatom; the term “arylene” as used herein refers to a carbocyclic aromatic moiety, which may consist of 1 or more rings fused together; the term “heteroarylene” as used herein refers to a carbocyclic aromatic moiety, which may consist of 1 or more rings fused together and wherein at least one carbon in one of the rings is replaced by a heteroatom; the term “cycloalkylene” as used herein refers to a saturated or unsaturated non-aromatic carbocycle moiety, which may consist of 1 or more rings fused together; the term “heterocycloalkylene” as used herein refers to a non-aromatic cyclic hydrocarbon moiety which may consist of 1 or more rings fused together and wherein at least one carbon in one of the rings is replaced by a heteroatom. Exemplary multivalent moieties include those examples given for the monovalent groups hereinabove in which one or more hydrogen atoms are removed.

Cyclic substructures in a linker reduce the molecular flexibility as measured by the number of rotatable bonds, which leads to a better membrane permeation rate, important for all in vivo cell culture labeling applications.

### Substrate Specificity of hmC-CT for Modified Cytosines in Nucleic Acids

The hmC-CT was shown to preferentially reacts with the hydroxyl group on 5-hmC on single stranded DNA, RNA or free nucleoside triphosphates in vitro to form a cmC (see for example, FIG. 7A-7F).

Relatively little carbamoyl conversion of 5-hmC in double stranded DNA was observed. In contrast, more than 60%, 70%, 80% or 90% of 5-hmC in single stranded DNA was converted into 5-cmdC in the denatured T4gt genomic DNA (see for example FIG. 7B). hmC-CT was also able to modify free deoxynucleoside triphosphate to form 5-hmdCTP with greater than 50% efficiency. hmrC in RNA could also be carbamoylated as could 5-hmrCTP (see for example, FIGS. 7E-7F and FIGS. 8A-8C).

### HmC-CT does not have a Significant Preference for Particular Sequence Contexts

All combinations of NCN motif containing 5-hmdC displayed comparable modification ratios and no significantly preferred motifs were observed, suggesting a general binding mechanism by hmC-CT.

As illustrated by FIG. 7D, carbamoylation protects cytosine derivative from deamination by APOBEC in the 16 different triplet sequence contexts tested in the denatured T4gT genome (5-hmdC) where the difference in deamination rate between control and treated libraries was indicative of carbomylation (see also Example 3).

### Uses of hmC-CT and Variants Thereof for Adding a Carbamoyl Group onto hmC or hmCTPs

There are many uses for using hmC-CT to add a carbamoyl group on to hmC either as a nucleoside triphosphate or in a nucleic acid. These uses generally fall into two categories. The first includes methods for modifying existing nucleic acids while the second category is for in vitro or in vivo synthesis of modified nucleic acids de novo. In some embodiments, the hmC is carbamoylated with carbamoyl phosphate. In other embodiments, the carbamoyl phosphate may be tagged with a chemically reactive group or may be tagged with a functional group attached directly or through the chemically reactive group either via a linker or directly. Where the carbamoyl phosphate contains an additional chemically reactive group only prior to carbamoylation to the hmC, the opportunity exists to add a functional group of choice after carbamoylation. This may be preferred for methods of synthesis of modified nucleic acids de novo.

Where an hmC is labelled in a nucleic acid, it may be desirable to use a carbamoyl phosphate substrate with hmC-CT to easily enable downstream manipulation of the nucleic acid.

Tagged carbamoyl phosphate for modification of nucleic acids or nucleoside triphosphates having a functional group may be especially useful for enriching, stabilizing, detecting or sequencing target molecules.

### Detecting Modified Bases in Eukaryotic Derived Nucleic Acids

As described above, carbamoyl phosphate can readily be combined with a chemically reactive groups used in click chemistry before or after its use as a substrate for the hmC-CT and its attachment to hmC via the phosphate group. These compounds enable the attachment of functional groups, for example, a fluorescent group for visualization of the cmC. Alternatively or in addition, an affinity binding domain such as biotin can be added to the carbamoyl group for attaching the nucleic acid to a solid substrate for purposes of enrichment. Bulky functional groups may be selected to facilitate sequencing methods used on various sequencing platforms such as the Pacific Biosystems whole genome sequencing platform or other nanopore sequencing methods where a bulky group on the hmC can trigger an enhanced signal that can unambiguously record the presence of the hmC by the sequencing platform. This may assist in the sequencing of smaller amounts of nucleic acid than might otherwise be possible. Other functional groups may include RNA stabilizing ligands for use in RNA therapeutics and vaccines where RNA stability is a desirable feature.

FIG. 10A shows examples of commercial compounds used for Click chemistry have been transferred onto a carbamoyl phosphate and 10B shows the same molecules linked through an oxymethylcytosine. The examples in FIGS. 10A-10B include azido or alkyne groups on alkyl or PEG linkages that are linked directly to R1 or R2 of the carbamoyl phosphate. Examples shown are also provided for various DBCO side groups that are cyclo-octines containing a reactive triple bond. These DBCO reactive groups may be linked via a linkage group (in this case PEG) to the carbamoyl phosphate at the R1 or R2 position. A sulfo group may be added to enhance solubility of the complex. Accordingly some of the compounds shown in FIG. 10B have a sulfite group as shown (see for example, sullfo DBCO PEG carbamoyl phosphate.

Where carbamoyl phosphate is used for enrichment of nucleic acids with modified cytosine, it may be useful to include a photocleavable linkage to release the enriched nucleic acid from a substrate. An example of a photocleavable linkage is also provided on DBCO in FIGS. 10A and 10B.

Tetrazine, methyl tertazine and TCO are commercial chemical compounds also used in Click chemistry that are shown here to be linked via PEG to carbamoyl phosphate (FIG. 10A) or via the carbamoyl group to cytosine (FIG. 10B). hmC-CT can be used in molecular biology workflows to generate cmC in DNA or RNA and nucleotide triphosphates. This has one or more of the following applications:

(a) Detection of hmC in a nucleic acid:

Detection of modified nucleotides in large genomic fragments or RNAs is facilitated by carbamoylation of hmC with a carbamoyl phosphate substrate. Additionally, a tag can be added to the carbamoyl phosphate substrate prior to carbamoylation resulting in a tagged cmC in the nucleic acid. Sequencing platforms such as Pacific Biosystems sequencers and nanopore sequencers (such as the Oxford nanopore sequencer) may more readily detect cmC or tagged cmC than unreacted hmC in a nucleic acid sequence thereby facilitating sequencing of DNA optionally without an amplification step.

Nucleic acids that have been released from a prokaryotic or eukaryotic cell or viruses that contain hmC can similarly be carbamoylated in vitro or can be carbamoylated in situ in a cell or particle for histological analysis using tagged carbamoyl phosphate reagents with the hmC-CT. In these circumstances, the tag on the carbamoyl phosphate may be a colorimetric or fluorescent dye that enables modified nucleotides to be visualized in the cells or particles under a microscope.

(b) Immobilization of carbamoylated nucleic acids

The addition of an affinity binding moiety through R1 and/or R2 on a carbamoyl phosphate shown in Formula 1 enables a carbamoylated nucleic acid to become bound to an affinity substrate. This has advantages for enrichment of nucleic acid molecules containing nucleic acid modifications. If desired, nucleic acids with different numbers of nucleotide modifications may be separated from each other by altering binding conditions such that nucleic acids with fewer modifications over a defined length of a nucleic acid will be eluted while nucleic acids with a greater number of modifications will remain bound (see for example US 8,980,553 and US 9,145,580 for enrichment of methylated double stranded DNA using a methyl-binding domain). In one embodiment, the more common methylated nucleotides in an isolated target nucleic acid may be oxidized with a mC dioxygenase such as a TET enzyme, and subsequently denatured, carbamoylated and immobilized on an affinity column (see section above on R1 and R2 modifications). In another embodiment, single stranded DNA and/or RNA that may circulate in a body fluid such as blood or is part of an in vitro or in vivo diagnostic workflow, may be reacted with the mC dioxygenase that oxidize single stranded DNA and RNA, and with hmC-CT and carbamoyl phosphate linked to an affinity binding moiety or reactive with an affinity binding moiety resulting in the addition of the affinity binding moiety to hmC.

In one embodiment, an affinity binding molecule may be added to the cmC or the carbamoyl phosphate prior to its reaction with hmC in a DNA or RNA present for example in extracellular fluid from a mammalian subject to enrich the sample containing hmC.

(c) Stabilizing a nucleoside triphosphate and/or stabilizing a single stranded nucleic acid:

Single strand nucleic acids including oligonucleotides are used in a plethora of different contexts. Improvements in stabilizing single strand nucleic acids is desirable. For example, RNA now forms a significant part of treatment options for infectious diseases exemplified by COVID vaccine production and this requires that the RNA is stable. Other examples of single stranded nucleic acids and oligonucleotides in workflows include: oligonucleotides that reversibly inhibit enzyme, oligonucleotides that can stabilize lyophilization of Taq polymerase, oligonucleotides that act as splints for analyzing microRNAs, oligonucleotides that act as primers, probes, or adaptors, oligonucleotides in arrays for sequencing, oligonucleotides that act as guides for cleavage enzymes (e.g. CRISPR) or as activator molecules for restriction endonucleases (such as MspJl or PaqCl), oligonucleotides that can serve as a leader sequence in Oxford nanopore sequencing where a carbamoylated nucleotide can be placed at the terminal nucleotide of the leader sequence marking the end of the artificial sequence and the beginning of the nucleic acid sequence of interest, etc. In one embodiment, it is desirable to stabilize these nucleic acid or oligonucleotide reagents for storage at suitable temperatures such as room temperature and to improve the shelf life profile of the reagents by carbamoylation with a carbamoyl phosphate or tagged carbamoyl phosphate where the tag is selected from those listed herein.

(d) Mapping methylated and hydroxymethylated nucleotides in nucleic acids in a single sequencing event

In one embodiment, detecting methylated and hydroxymethylated cytosine in nucleic acids may be achieved by initially labeling hmC in a double stranded nucleic acid by adding a glucose or derivative thereof with a GT such as BGT to form glucosylated hydroxymethylcytosine (ghmC) and in a second aliquot converting mC to unlabeled hmC with TET before denaturation into single stranded DNA, and labeling the hmC with a carbamoyl group. A deaminase can be used to convert cytosine to uracil and any mC to thymine for comparative purposes.

It is also possible to label an aliquot of the nucleic acid with carbamoyl phosphate or a tagged carbamoyl phosphate and a second aliquot, combining TET with BGT to label hmC in the nucleic acid with a glucose or derivative thereof via a GT and comparing the sequences of the 2 aliquots.

Using a large molecule sequencer such as PacBio or Oxford Nanopore, ghmC and cmC can be mapped by direct sequencing.

### Method of use of hmC-CT and Carbamoyl Phosphate or Tagged Carbamoyl Phosphate Substrates in the de novo Synthesis of Nucleic Acids with Modified Cytosine

The nucleic acid may include one or more modified nucleotides including unnatural nucleotides. Chemical modification of nucleic acids is a widely used strategy for optimization of their biological activity and potency, such as target binding affinity, duplex conformation, hydrophobicity, stability, nuclease resistance, and immunostimulatory properties. Chemical modification can confer unique properties to oligonucleotides or oligonucleotide conjugates. Some chemically modified nucleotides can be incorporated into oligonucleotides to crosslink them to DNA, RNA or proteins upon exposure to UV light (e.g., 5-bromo-dU). Some chemically modified nucleotides are duplex-stabilizing modifications and can be incorporated into oligonucleotides to increase the oligonucleotide Tm (e.g., Super T). Some nucleobase modifications confer additional fluorescent properties oligonucleotides. (e.g., 2-aminopurine). Some modified nucleobases, also known as universal bases, do not favor any particular base-pairing and enable random incorporation of any specific base during amplification (e.g., 5-nitroindole). Modifications of the 2′-sugar position (e.g., 2′-methyl and 2′-methoxyethyl) promote the A-form or RNA-like conformation in oligonucleotides, considerably increasing their binding affinity to RNA, and having enhanced nuclease resistance. The 2′-modification can reduce oligonucleotide immunostimulatory and off-target effects. Some modified nucleotides can trigger RNAse H activity (e.g., oxepane nucleic acids, ONA). Oligonucleotides comprising bridged rings (also known as bridged nucleic acids, e.g., Locked nucleic acids, LNAs) lock the base in the C3′-endo position, favoring RNA A-type helix duplex geometry, increasing Tm and nuclease resistance. Modifications of the oligonucleotide backbone (e.g., a phosphororothioate linkage) have been used to increase the resistant oligonucleotides to exo-and endonucleases. Oligonucleotides comprising backbone modifications have been widely used as antisense reagents or in synthetic siRNA for the control of gene expression. Examples and uses of oligonucleotide chemical modifications are reviewed in a variety of publications, such as in Deleavey, et al, Chemistry & Biology 2012, 19(8): 937-54.

Nucleic acids may be synthesized that contain carbamoylated mC by methods that include (a) synthesizing the nucleic acid chemically or enzymatically from a pool of nucleotides that include cmC; or (b) synthesizing nucleic acids containing hmC and then reaction the hmC with hmC-CT to transfer a carbamoyl group onto the mC via the hydroxyl group (Reese, Organic & Biomolecular Chemistry. 3 (21): 3851-68 (2005)).

The carbamoyl group is relatively stable and is not degraded or substantially affected by the chemical synthesis reaction. Hence carbamoylated precursors behave just like another nucleotide in chemical synthesis. Methods of chemical synthesis of oligonucleotides are well established.

Oligonucleotide synthesis is commonly carried out by a stepwise addition of nucleotide residues to the 5′-terminus of the growing chain until the desired sequence is assembled.

For enzymatic synthesis, a DNA polymerase, RNA polymerase or reverse transcriptase can be used to incorporate the carbamoylated dNTP or rNTP into nucleic acid, The carbamoyl modification at the 5-position of cytosine does not affect Watson-Crick base pairing and therefore does not substantially affect the ability of polymerases to incorporate the modified nucleotide.

Synthesis of nucleic acids that include carbamoylated mC can be facilitated by tags that may be bound to the carbamoylated mC that may facilitate enrichment of the desired nucleic acid through affinity binding of the tag to a suitable substrate. Carbamoylated mC in the synthesized nucleic acids may aid in visualizing the progress of synthesis and in quality control in terms of sequence integrity of the synthesized nucleic acids.

Synthesized nucleic acids containing carbamoylated mC that are optionally tagged have a number of uses such as (a) for aptamers to enhance stability of the nucleic acids used for example in inhibiting enzyme activity of various enzymes such as polymerases or nucleases at non-reaction temperatures; (b) for guide nucleic acids used in directed cleavage of genomic DNA in combination with CrisPR associated proteins (Cas), (c) for primers and adapters where these may be tagged to adhere or become linked to a solid substrate such as a bead or form an array, for use in linkers for circularizing DNA or RNA prior to amplification and/or sequencing.

In certain embodiments, it may not be necessary or desirable to carbamoylate every cytosine in a nucleic acid molecule in which case the extent of carbamoylation may be regulated by the amount of hmdCTP or hmrCTP ratio to dCTP or rCTP in the nucleotide pool prior to a nucleic acid synthesis reaction.

In other embodiments, it may be desirable to have a plurality of different tags in a synthesized nucleic acid. Accordingly a mixture of different tagged carbamoyl phosphate substrates may be combined with the hmC-CT to react with the pool of hmdCTP, or hmrCTP prior or during synthesis of the nucleic acid.

hmC-CT and carbamoyl substrates may be used for pulse chasing in Eukaryotic cells. For example, changes in methylation or hydroxymethylation in a genome may be tracked using this enzyme and substrate.

TABLE 3
Sequence positions for sequences listed in FIG. 9A-9D and in
the full sequence listing for the SEQ ID NO as indicated.

| SEQ ID NO | Positions |
|---|---|
| SEQ ID NO: 1 | FIG. 9A |
| | NEBs3_Carbam_trans_C |
| | Position 383-535 |
| | FIG. 9B |
| | NEBs3_Carbam_trans_N |
| | Position 101-330 |
| | FIG. 9C |
| | NEBs3_Carbam_trans_C |
| | Position 383-535 |
| | FIG. 9D |
| | NEBs3_Carbam_trans_N |
| | Position 162-183 |
| SEQ ID NO: 29 | FIG. 10C - Whole Sequence |
| | FIG. 9A |
| | Modified_001 |
| | Position 373-522 |
| | FIG. 9B |
| | NEBs1_Carbam_trans_N |
| | Position 101-325 |
| | FIG. 9C |
| | NEBs1_Carbam_trans_C |
| | Position 373-520 |
| | FIG. 9D |
| | NEBs1_Carbam_trans_N |
| | Position 160-178 |
| SEQ ID NO: 30 | FIG. 9A |
| | Modified_002 |
| | Position 382-531 |
| | FIG. 9B |
| | Modified_002 |
| | Position 22-56 |
| | Modified_002 |
| | Position 108-335 |
| | FIG. 9C |
| | Modified_002 |
| | Position 382-530 |
| | FIG. 9D |
| | Modified_002 |
| | Position 170-190 |
| SEQ ID NO: 31 | FIG. 9A |
| | Modified_003 |
| | Position 340-490 |
| | FIG. 9B |
| | Modified_003 |
| | Position 67-194 |
| | FIG. 9C |
| | Modified_003 |
| | Position 340-492 |
| | FIG. 9D |
| | Modified_003 |
| | Position 128-149 |
| SEQ ID NO: 32 | FIG. 9A |
| | Modified 004 |
| | Position 402-539 |
| | FIG. 9B |
| | Modified_004 |
| | Position 161-283 |
| | FIG. 9D |
| | Modified_004 |
| | Position 225-245 |
| SEQ ID NO: 33 | FIG. 9A |
| | Modified_005 |
| | Position 385-533 |
| | FIG. 9B |
| | Modified_005 |
| | Position 107-232 |
| | FIG. 9C |
| | Modified_005 |
| | Position 385-533 |
| | FIG. 9D |
| | Modified_005 |
| | Position 169-190 |
| SEQ ID NO: 34 | FIG. 9A |
| | Modified_008 |
| | Position 373-430 |
| | FIG. 9B |
| | Modified_008 |
| | Position 8-61 |
| | Modified_008 |
| | Position 109-318 |
| | FIG. 9C |
| | Modified_008 |
| | Position 373-431 |
| | FIG. 9D |
| | Modified_008 |
| | Position 152-173 |
| SEQ ID NO: 35 | FIG. 9A |
| | Modified_010 |
| | Position 277-425 |
| | FIG. 9B |
| | Modified_010 |
| | Position 5-128 |
| | FIG. 9C |
| | Modified_010 |
| | Position 277-425 |
| | FIG. 9D |
| | Modified_010 |
| | Position 64-84 |
| SEQ ID NO: 36 | FIG. 9A |
| | Modified_006 |
| | Position 375-522 |
| | FIG. 9B |
| | Modified_006 |
| | Position 1-326 |
| | FIG. 9C |
| | Modified_006 |
| | Position 375-522 |
| | FIG. 9D |
| | Modified_006/154-175 |
| | Position 154-175 |
| SEQ ID NO: 37 | FIG. 9A |
| | Modified_007 |
| | Position 373-524 |
| | FIG. 9B |
| | Modified_007 |
| | Position 94-330 |
| | FIG. 9C |
| | Modified_007 |
| | Position 373-524 |
| | FIG. 9D |
| | Modified_007 |
| | Position 161-182 |
| SEQ ID NO: 38 | FIG. 9B |
| | Modified_n_018 |
| | Position 17-93 |
| | Modified_n_018 |
| | Position 102-312 |
| | FIG. 9D |
| | Modified_n_018 |
| | Position 159-180 |
| SEQ ID NO: 39 | FIG. 9A |
| | Modified_014 |
| | Position 348-421 |
| | FIG. 9B |
| | Modified_014 |
| | Position 61-93 |
| | Modified_014 |
| | Position 102-312 |
| | FIG. 9C |
| | Position |
| | Modified_014 |
| | FIG. 9D |
| | Modified_014 |
| | Position 159-180 |
| SEQ ID NO: 40 | FIG. 9B |
| | Modified_n_019 |
| | Position 17-101 |
| | Modified_n_019 |
| | Position 103-285 |
| | FIG. 9C |
| | Modified_n_019 |
| | Position 142-163 |
| | FIG. 9D |
| | Modified_n_019 |
| | Position 160-180 |
| SEQ ID NO: 41 | FIG. 9A |
| | Modified_013 |
| | Position 375-524 |
| | FIG. 9B |
| | Modified_013 |
| | Position 8-59 |
| | Modified_013 |
| | Position 86-316 |
| | FIG. 9C |
| | Modified_013 |
| | Position 375-524 |
| | FIG. 9D |
| | Modified_013 |
| | Position 152-173 |
| SEQ ID NO: 42 | FIG. 9A |
| | Modified_015 |
| | Position 189-322 |
| | FIG. 9B |
| | Modified 015 |
| | Position 1-152 |
| | FIG. 9C |
| | Modified_015 |
| | Position 189-324 |
| | FIG. 9D |
| | Modified_015 |
| | Position 1-20 |
| SEQ ID NO: 43 | |
| SEQ ID NO: 44 | Modified_n_020 |
| | Position 106-282 |
| SEQ ID NO: 45 | |
| SEQ ID NO: 46 | FIG. 9A |
| | Modified_011 |
| | Position 396-554 |
| | FIG. 9B |
| | Modified_011 |
| | Position 101-346 |
| | FIG. 9C |
| | Modified_011 |
| | Position |
| | FIG. 9D |
| | Modified_011 |
| | Position 176-197 |
| SEQ ID NO: 47 | FIG. 9A |
| | Modified_012 |
| | Position 373-521 |
| | FIG. 9B |
| | Modified_012 |
| | Position 11-51 |
| | Modified_012 |
| | Position 103-327 |
| | FIG. 9C |
| | Modified_012 |
| | Position 373-521 |
| | FIG. 9D |
| | Modified_012 |
| | Position 154-175 |
| SEQ ID NO: 48 | FIG. 9A |
| | TobZ_Carbam_trans_C |
| | Position 403-561 |
| | FIG. 9B |
| | TobZ_Carbam_trans_N |
| | Position 113-354 |
| | FIG. 9C |
| | TobZ_Carbam_trans_C |
| | Position 403-576 |
| | FIG. 9D |
| | TobZ_Carbam_trans_N |
| | Position 171-191 |
| SEQ ID NO: 49 | FIG. 9C |
| | YP_239307.1 |
| | Position 379-547 |
| | FIG. 9D |
| | YP_239307.1 |
| | Position 159-180 |

Unmodified 001 corresponds to SEQ ID NO: 50, Unmodified 002 corresponds to SEQ ID NO: 51,
Unmodified 003 corresponds to SEQ ID NO: 52, Unmodified 004 corresponds to SEQ ID NO: 53,
Unmodified 005 corresponds to SEQ ID NO: 54, Unmodified 006 corresponds to SEQ ID NO: 55,
Unmodified 007 corresponds to SEQ ID NO: 56, Unmodified 008 corresponds to SEQ ID NO: 57,
Unmodified 009 corresponds to SEQ ID NO: 58, Unmodified 010 corresponds to SEQ ID NO: 59,
Unmodified 011 corresponds to SEQ ID NO: 60, Unmodified 012 corresponds to SEQ ID NO: 61,
Unmodified 013 corresponds to SEQ ID NO: 62, Unmodified 014 corresponds to SEQ ID NO: 63,
Unmodified 015 corresponds to SEQ ID NO: 64, Unmodified 016 corresponds to SEQ ID NO: 65,
Unmodified 017 corresponds to SEQ ID NO: 66, Unmodified 018 corresponds to SEQ ID NO: 67,
Unmodified 019 corresponds to SEQ ID NO: 68, Unmodified 020 corresponds to SEQ ID NO: 69,
Unmodified 021 corresponds to SEQ ID NO: 70, Unmodified 022 corresponds to SEQ ID NO: 71,
Unmodified 023 corresponds to SEQ ID NO: 72, Unmodified 024 corresponds to SEQ ID NO: 73,
Unmodified 025 corresponds to SEQ ID NO: 74, Unmodified 026 corresponds to SEQ ID NO: 75,
Unmodified 027 corresponds to SEQ ID NO: 76, Unmodified 028 corresponds to SEQ ID NO: 77,
Unmodified 029 corresponds to SEQ ID NO: 78, Unmodified 030 corresponds to SEQ ID NO: 79,
Unmodified 031 corresponds to SEQ ID NO: 80, Unmodified 032 corresponds to SEQ ID NO: 81,
Unmodified 033 corresponds to SEQ ID NO: 82, Unmodified 034 corresponds to SEQ ID NO: 83.

### General CONSIDERATIONS

Unless otherwise defined, all technical and scientific terms used herein have the same meaning as commonly understood by one of ordinary skill in the art to which this disclosure belongs. Still, certain terms are defined herein with respect to embodiments of the disclosure and for the sake of clarity and ease of reference.

Sources of commonly understood terms and symbols may include: standard treatises and texts such as Kornberg and Baker, DNA Replication, Second Edition (W.H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Eckstein, editor, Oligonucleotides and Analogs: A Practical Approach (Oxford University Press, New York, 1991); Gait, editor, Oligonucleotide Synthesis: A Practical Approach (IRL Press, Oxford, 1984); Singleton, et al., Dictionary of Microbiology and Molecular biology, 2d ed., John Wiley and Sons, New York (1994), and Hale & Markham, the Harper Collins Dictionary of Biology, Harper Perennial, N.Y. (1991) and the like.

As used herein and in the appended claims, the singular forms “a”, “an”, and “the” include plural referents unless the context clearly dictates otherwise. For example, the term “a protein” refers to one or more proteins, i.e., a single protein and multiple proteins. The claims can be drafted to exclude any optional element when exclusive terminology is used such as “solely,” “only” are used in connection with the recitation of claim elements or when a negative limitation is specified.

Aspects of the present disclosure can be further understood in light of the embodiments, section headings, figures, descriptions and examples, none of which should be construed as limiting the entire scope of the present disclosure in any way. Accordingly, the claims set forth below should be construed in view of the full breadth and spirit of the disclosure.

Each of the individual embodiments described and illustrated herein has discrete components and features which may be readily separated from or combined with the features of any of the other several embodiments without departing from the scope or spirit of the present teachings. Any recited method can be carried out in the order of events recited or in any other order which is logically possible. Numeric ranges are inclusive of the numbers defining the range. All numbers should be understood to encompass the midpoint of the integer above and below the integer i.e., the number 2 encompasses 1.5-2.5. The number 2.5 encompasses 2.45-2.55 etc. When sample numerical values are provided, each alone may represent an intermediate value in a range of values and together may represent the extremes of a range unless specified.

In the context of the present disclosure, “non-naturally occurring” refers to a polynucleotide, polypeptide, carbohydrate, lipid, or composition that does not exist in nature. Such a polynucleotide, polypeptide, carbohydrate, lipid, or composition may differ from naturally occurring polynucleotides polypeptides, carbohydrates, lipids, or compositions in one or more respects. For example, a polymer (e.g., a polynucleotide, polypeptide, or carbohydrate) may differ in the kind and arrangement of the component building blocks (e.g., nucleotide sequence, amino acid sequence, or sugar molecules). A polymer may differ from a naturally occurring polymer with respect to the molecule(s) to which it is linked. For example, a “non-naturally occurring” protein may differ from naturally occurring proteins in its secondary, tertiary, or quaternary structure, by having a chemical bond (e.g., a covalent bond including a peptide bond, a phosphate bond, a disulfide bond, an ester bond, and ether bond, and others) to a polypeptide (e.g., a fusion protein), a lipid, a carbohydrate, or any other molecule. Similarly, a “non-naturally occurring” polynucleotide or nucleic acid may contain one or more other modifications (e.g., an added label or other moiety) to the 5′- end, the 3′ end, and/or between the 5′- and 3′-ends (e.g., methylation) of the nucleic acid. A “non-naturally occurring” composition may differ from naturally occurring compositions in one or more of the following respects: (a) having components that are not combined in nature, (b) having components in concentrations not found in nature, (c) omitting one or components otherwise found in naturally occurring compositions, (d) having a form not found in nature, e.g., dried, freeze dried, crystalline, aqueous, and (e) having one or more additional components beyond those found in nature (e.g., buffering agents, a detergent, a dye, a solvent or a preservative).

All publications, patents, and patent applications mentioned in this specification are herein incorporated by reference to the same extent as if each individual publication, patent, or patent application was specifically and individually indicated to be incorporated by reference, including U.S. Provisional Ser. No. 63/151,378 filed Feb. 19, 2021, and U.S. Provisional Application Ser. No. 63/151,400 filed Feb. 19, 2021.

### EMBODIMENTS

Embodiment 1. A kit comprising hydroxymethylcytosine carbamoyltransferase (hmC-CT), and at least one of carbamoyl phosphate, and in the same or separate containers, one or more reagents selected from carbamoyl phosphate, a TET family enzyme or mutant thereof, a glucosyltransferase (GT), a deaminase, and a helicase.

Embodiment 2. A composition comprising a fusion protein, wherein one portion of the fusion protein is a portion of a hmC-CT and a second portion of the fusion is an affinity binding domain or a DNA binding protein.

Embodiment 3. The composition according to embodiment 2, wherein the affinity binding domain is selected from the group consisting of biotin or desthiobiotin, maltose binding protein, methyl binding protein, chitin binding protein, SNAP-tag, antibody or fragment thereof, and Proteinase K or variant thereof.

Embodiment 4. The composition according to embodiment 2 or 3, wherein the fusion protein is immobilized on a matrix.

Embodiment 5. The composition according to embodiment 4, wherein the matrix is a magnetic bead.

Embodiment 6. A composition comprising lyophilized hmC-CT.

Embodiment 7. A composition comprising hmC-CT In a storage buffer containing at least 30%, 40% or 50% glycerol.

Embodiment 8. The composition according to any of embodiments 2-7, further comprising an oligonucleotide for enhancing or depressing the activity of the hmC-CT in the presence of carbamoyl phosphate and a substrate nucleic acid or altering its specificity for modifying nucleotides in the substrate nucleic acid.

Embodiment 9. The composition according to any of embodiments 2-8, wherein the hmC-CT has at least 80% or 90% sequence identity to SEQ ID NO:1.

Embodiment 10. A composition comprising a modified carbamoyl phosphate, wherein the modification is selected from one or more moieties consisting of a linker, a detectable moiety, an isolation tag, a blocking moiety, and a functional moiety.

Embodiment 11. The composition according to embodiment 10, further comprising a hmC-CT.

Embodiment 12. A method for distinguishing 5-hydroxymethylcytosine (5-hmC) from 5-methylcytosine (5-mC) in a nucleic acid molecule comprising:
(a) placing in a reaction mixture: the target nucleic acid molecule; a hmC-CT (hmC-CT) and carbamoyl phosphate (CP); and
(b) modifying hmC in the nucleic acid molecule to form a 5-carbamoyloxymethylcytosine (5-cmC).

Embodiment 13. The method of embodiment 12, further comprising: detecting 5-hydroxymethylated deoxycytosine (5-cmdC) or 5-hydroxymethylated ribocytosine (5-cmrC) in the nucleic acid molecule.

Embodiment 14. The method according to embodiment 12, wherein the carbamoyl phosphate comprises one or more moieties selected from the group consisting of: a linker, a detectable moiety, an isolation tag, a blocking moiety, and a functional moiety.

Embodiment 15. The method according to embodiment 12, further comprising: enriching for the nucleic acid having 5-carbamoyloxymethylcytosine (5-cmC) by means of an affinity tag on one of: the carbamoyl phosphate, hmC-CT, or nucleic acid substrate.

Embodiment 16. The method according to embodiment 15, wherein the nucleic acid in the reaction mixture is enriched by immobilization on a matrix.

Embodiment 17. The method according to embodiment 10, wherein the nucleic acid is single stranded.

Embodiment 18. The method according to embodiment 17, wherein the nucleic acid is chromosomal DNA and/or mRNA and optionally using dye tagged carbamoyl phosphate to detect the location of 5-hydroxymethylcytosine (5-hmC) in vivo or in vitro.

Embodiment 19. The method according to embodiment 18, wherein the dye is selected from a fluorescent dye or a color dye.

Embodiment 20. The method according to any of embodiments 12-19, further comprising (c) amplifying the nucleic acid.

Embodiment 21. The method according to any of embodiments 12-20, further comprising sequencing the nucleic acid.

Embodiment 22. A method for obtaining nucleic acid modifying enzymes, comprising:
(a) obtaining phage nucleic acid from an environmental sample from which phage particles have been enriched;
(b) identifying whether the phage nucleic acid has modified nucleotides;
(c) performing a contig analysis of the phage nucleic acid for sequences encoding enzymes capable of modifying the phage nucleic acid; and

Embodiment 23. A method for determining the presence of nucleic acid modifications in low input nucleic acid samples obtained from a biological fluid or a cell lysate, wherein the method comprises:
(a) adding a carbamoyl group to hydroxymethylcytosines (hmCs); and
(b) detecting the presence of carbamoyl methylcytosine (cmC) in the nucleic acid. Embodiment 24. The method according to embodiment 23, wherein (a) further comprises: combining the nucleic acid from the low input sample with carbamoyl phosphate and hmC-CT. Embodiment 25. The method according to any of embodiments 23 and 24, wherein the biological fluid is selected from the group consisting of: blood, urine, sputum, mucous, feces, and spinal fluid of human patients.

Embodiment 26. The method according to embodiment 25, wherein the biological fluid is blood and low input nucleic acids is from exosomes.

Embodiment 27. The method according to embodiment 25, wherein the biological fluid is blood and the low input nucleic is maternal and fetal nucleic acids.

Embodiment 28. The method according to any of embodiments 23-27, wherein (a) further comprises enriching the low input nucleic in the biological fluid or cell lysate by immobilizing the nucleic acids on a matrix before or after adding the carbamoyl group to the hmC.

Embodiment 29. The method according to embodiment 28, wherein the matrix is a bead, a multi-well plastic dish or a paper.

Embodiment 30. The method according to any of embodiments 23-29, further comprising amplifying and/or sequencing the nucleic acids for detecting the presence of the cmC. Embodiment 31. The method of embodiment 23, wherein the 5-carbamoyloxymethyldeoxyribocytosine (5-cmdC) is detectable by means of liquid chromatography-mass spectrometry.

Embodiment 32. The method of any of embodiments 23-31, further comprising determining a phenotype from the detected 5-carbamoyloxymethyldeoxyribocytosine (5-cmdC).

Embodiment 33. A method, comprising:
(a) obtaining single stranded nucleic acid from a biological sample;
(b) adding a carbamoyl blocking group to some or all 5-hydroxymethylcytosine (5-hmC) in the single strand nucleic acid sample; and
(c) oxidizing the 5-methylcytosine (5-mC) in the sample to 5-hydroxymethylcytosine (5-hmC) and repeating (b).

Embodiment 34. The method according to embodiment 33, wherein the single stranded nucleic acid from the biological sample is a low input DNA sample.

Embodiment 35. The method according to embodiment 34, wherein the low input DNA is less than 100 ng, 10 ng, 1 ng or 100 pg.

Embodiment 36. The method according to embodiment 33, wherein the single stranded nucleic acid from the biological sample is fragmented and denatured double stranded DNA.

Embodiment 37. The method according to embodiment 33, further comprising one or more of the following steps selected from the group consisting of: (i) adding a linking group to the carbamoyl phosphate for forming 5-carbamoyloxymethyldeoxyribocytosine (5-cmdC) or 5-carbamoyloxymethylribocytosine (5-cmrC) in (b); (ii) ligating DNA adapters to the nucleic acid sample before (a), before or after (b) or before or after (c); (iii) adding an affinity tag to the linking group; enriching for the affinity tagged nucleic acid by affinity purification; (iv) amplifying the enriched DNA; and (v) sequencing the carbamoylated nucleic acid.

Embodiment 38. The method of embodiment 37, wherein one or more of the DNA adapters contain a unique molecular index sequence.

Embodiment 39. A method comprising: reacting a nucleic acid in a sample sequentially or in parallel with a first and second blocking group such that 5-hydroxymethylcytosine (5-hmC) is converted to a modified 5-hmC using one blocking group and 5-methylcytosine (5-mC) is modified with another blocking group so that both 5-mC and 5-hmC can be detected from a single sequence reaction. Embodiment 40. The method according to embodiment 39, wherein one blocking group is a carbamoyl group and another blocking group is glucose.

Embodiment 41. A method for determining the location of modified cytosines (C) in a nucleic acid in a sample, comprising:
(a) reacting an aliquot of the sample containing double stranded nucleic acid with (i) a GT for adding a sugar to 5-hydroxymethylcytosine (5-hmC), followed by (ii) a TET protein for oxidation of 5-methylcytosine (5-mC) and (iii) denaturing the nucleic acid into single strands and reacting the single stranded nucleic acid with a carbamoyltransferase (hmC-CT) in the presence of a carbamoyl salt; and
(b) sequencing the glucosylated and carbamoylated single strand nucleic acid to determine which cytosines in the initial nucleic acid are unmodified or modified by a methyl or hydroxymethyl group.

Embodiment 42. The method according to embodiment 41, further comprising performing (a) in a single tube.

Embodiment 43. The method according to embodiment 41, wherein the hmC-CT is immobilized on a matrix for facilitating separation of the hmC-CT from the nucleic acid prior to addition of TET. Embodiment 44. The method according to any of embodiments 41-43, wherein an inhibitor of the hmC-CT is added prior to the addition of TET.

Embodiment 45. A method for determining the location of modified cytosines in a nucleic acid in a sample, comprising:
(a) reacting an aliquot of the sample in which the nucleic acid is single stranded with a hmC-CT;
(b) permitting any methylated cytosines in the nucleic acid sample to be oxidized by adding TET protein;
(c) reacting the oxidized carbamoyl nucleic acid with a complementary single strand nucleic acid to form a double stranded DNA for reacting with GT; and
(d) performing whole genome sequencing on double stranded nucleic acid to determine the location of 5-methylcytosine (5-mC) and 5-hydroxymethylcytosine (5-hmC) in the nucleic acid. Embodiment 46. The method according to embodiment 45, further comprising performing (a) in a single tube.

Embodiment 47. A method for determining the location of modified cytosines (C) in a nucleic acid in a sample, comprising:
(a) reacting an aliquot of the sample in which the nucleic acid is single stranded with a carbamoyltransferase;
(b) permitting the single stranded carbamoylated nucleic acid to reanneal to form double stranded nucleic acid and adding TET protein to oxidize any methylated cytosines in the nucleic acid sample;
(c) reacting the oxidized carbamoyl nucleic acid with a hmC-CT; and
(d) performing whole genome sequencing on double stranded nucleic acid to determine the location of the glucosylated nucleotides and the carbamoyl nucleotides in the nucleic acid sequence.

Embodiment 48. A synthetic oligonucleotide containing one or more carbamoylated methylcytosines (cmC).

Embodiment 49. The synthetic oligonucleotide according to embodiment 48, wherein the oligonucleotide is an aptamer.

Embodiment 50. The synthetic oligonucleotide according to embodiment 49, wherein the aptamer reversibly inhibits enzyme activity of a target enzyme.

Embodiment 51. The synthetic oligonucleotide according to embodiment 48, wherein the oligonucleotide is selected from one or more of: splint ligation of a single stranded DNA or RNA fragments; a guide RNA for directing a cleavage of a nucleic acid by means of an enzyme and a guide or activator oligonucleotide; a leader sequence for RNA sequencing; an RNA or single strand DNA in a particle formulated for a vaccine; or a member of a sequencing array.

### EXAMPLES

### Example 1: Methods used for embodiments of the invention

Genomic DNA. The E. coli, XP12 (5-mC) and T4gt (5-hmC) genomic DNA used in this study were obtained from New England Biolabs, Ipswich, MA.

Environmental phage collection. For each batch, 2 ˜ 4 liters of sewage or coastal seawater were used for phage collection. Large debris and bacterial cells were pelleted and removed by centrifuging at 5,000 xg for 30 minutes. Phage particles in the supernatant were precipitated by adding PEG8000 to 10% (w/v) and NaCl to 1 M and let stand at 4° C. overnight. Aggregates of phage particles were pelleted at 10,000 xg for 30 minutes, washed with 10% PEG8000 and 1 M NaCl solution, and resuspended in 2˜4 mL of phage dilution buffer (10 mM Tris-HCl at pH 8.0, 10 mM MgCl₂, 75 mM NaCl). The crude phage particle suspension was stored at 4° C. for subsequent phenol-chloroform DNA extraction.

Phenol-chloroform DNA extraction. 2˜4 mL of crude phage suspension was divided in 400 μL aliquots. For each aliquot, phage particles were lysed at 56° C. for 2 hours in 550 μL of lysis buffer (100 mM Tris-HCl at pH 8.0, 27.3 mM EDTA, 2% SDS, ˜1.6 U Proteinase K (New England Biolabs, Ipswich, MA). After lysis, RNase A was added to 10 μg/mL and incubated at 37° C. for 30 minutes. 1× volume (˜550 μL) of phenol-chloroform (Tris-HCl buffered at pH 8.0) was mixed with the lysis solution and vortexed vigorously for ˜1 minute and centrifuged at 10,000× g for 5 minutes for phase separation. The top aqueous layer (˜500 μL) was collected and mixed with 1× volume of chloroform, vortex vigorously, and centrifuged for phase separation. The top aqueous layer (˜450 μL) was collected. 1× volume of isopropanol was slowly added on top of the aqueous solution. Phage DNA was “spooled” with a glass capillary by swirling and mixing isopropanol with the aqueous solution. The spooled DNA was washed in 70% ethanol, dried at room temperature for ˜30 minutes, and dissolved in ˜600-800 μL of TE buffer (10 mM Tris pH 7.5, 1 mM EDTA).

The phage DNA solution was further purified by ethanol precipitation. Briefly, DNA was precipitated by adding 0.1X volume of 3 M sodium acetate and 2.5× volume of ethanol and incubated at −20° C. overnight. Precipitated DNA was pelleted at 16,000× g for 20 minutes, washed twice with 1 mL of 70% ethanol, dried at room temperature, and finally dissolved in 200 μL of TE buffer for storage at −20° C. On average more than 20 μg of DNA was extracted in each batch.

Illumina library preparation. For each library, 1 μg of phage metagenomic DNA was sheared to 300 bp in 130 μL of TE buffer (10 mM Tris pH 7.5, 1 mM EDTA) using Covaris S2 Focused Ultrasonicator (Covaris, Woburn, MA). 1.3 μL of 10 mg/mL RNase A (Qiagen, Germantown, MD) was added and incubated at 37ºC for 30 minutes to remove RNA. To remove EDTA, the sheared DNA was purified with Zymo Oligo Clean & Concentrator™ Kit (Zymo Research, Irvine, CA) and eluted in 50 μL of 1 mM Tris buffer (pH 7.5).

One reaction of NEBNext® Ultra™ II DNA Library Prep Kit for Illumina® (New England Biolabs, Ipswich, MA) was used for 1 μg of input DNA, with the following modification to the standard protocol: Pyrrolo-dC Y-shaped Illumina adaptors were used to protect the adaptor from subsequent enzymatic treatment. The DNA library was purified with 1× volume of NEBNext® Sample Purification Beads (New England Biolabs, Ipswich, MA) and eluted with 40 μL of 1 mM Tris buffer (pH 7.5).

For the two sewage DNA samples, each one contained two pairs of replicate libraries subjected to enzymatic selection or control respectively, The coastal sample generated only one pair: one library for enzymatic selection and one for control.

Enzymatic selection protocol. For each prepared library sample, 100 ng spiked-in genomic DNA mixture (E. coli:XP12:T4gt =1:1:1 by molarity) were added before being subjected to enzymatic selection. 1 L TET2 (New England Biolabs, Ipswich, MA) and 1 μL T4-BGT (New England Biolabs, Ipswich, MA) were added to the 50 μL reaction mixture containing 1× TET2 reaction buffer, 40 uM UDP-Glucose and 40 μM iron(ii) sulfate hexahydrate. After 60 minutes incubation at 37° C., Proteinase K was added at 0.4 mg/ml to inactivate the enzymes. Products were purified with Zymo Oligo Clean & Concentrator Kit and eluted in 16 μL water. To denature double stranded DNA, 4 μL formamide (Sigma-Aldrich, St. Louis, MO) was added. The 20 μL mixture was then incubated at 95° C. for 10 minutes and immediately transferred to an ice bath. One uL APOBEC (New England Biolabs, Ipswich, MA) was added directly to the reaction with 10 μL of 10x APOBEC reaction buffer and the reaction volume was brought up to 100 μL with water. APOBEC-mediated deamination was conducted at 37° C. for 3 hours. Purification was performed using Zymo Oligo Clean & Concentrator Kit and elution with 43 μL of water. In the final step, the library was incubated with 2 μL of USER (New England Biolabs, Ipswich, MA) in 1× CutSmart® Buffer (New England Biolabs, Ipswich, MA) at 37° C. for 15 minutes before final purification with Zymo Oligo Clean & Concentrator Kit.

Quantitative PCR. The qPCR reactions were performed with enzymatic selection or control samples using Luna® Universal qPCR Master Mix (New England Biolabs, Ipswich, MA) on a Bio-Rad CFX96™ Real-Time PCR Detection System (Hercules, CA). Two uL of purified DNA were added per reaction. Primers used in the experiments were the following: E. coli F: 5′-TTGCTGAGTTTCACGCTTGC (SEQ ID NO:18), E. coli R: 5′-AAAACCGCTTGTGGATTGCC (SEQ ID NO:19) , T4gt F: 5′-TCGCGAAACGGTTTTCCAAG (SEQ ID NO:20), T4gt R: 5′-AAAGCGCTTGACCCAACAAC (SEQ ID NO:21), XP12 F: 5′-TGCGATGTTGGATTCGTTGG (SEQ ID NO:22), and XP12 R: 5′-ACAACCCGCCATAATGGAAC (SEQ ID NO:23). Recovery was normalized to control using the delta-delta Ct method.

Illumina sequencing. Libraries were indexed, amplified using NEBNext® Ultra™ II Q5® Master Mix (New England Biolabs, Ipswich, MA) (6 cycles for control library and 12 cycles for selection library) and pooled for sequencing on an Illumina NextSeq® instrument (Illumina, San Diego, CA) with paired end reads of 75 bp.

Sequencing data processing. Paired-end reads were downloaded as FASTQ files and trimmed with Trim Galore v0.6.4 (https://www.bioinformatics.babraham.ac.uk/projects/trim_galore/) using the—paired option. K-mer counting from reads was done with JELLYFISH v2.2.10 and 16-mer was chosen based on best resolution. De novo assembly of contigs for each sample was performed with SPAdes v3.13.0 with the—meta option. We selectively reported contigs longer or equal to 1000 bp. To remove redundant contigs between selection and control pairs from each experiment, we used CD-HIT v4.8.1 nucleotide mode cd-hit-est with sequence identity threshold set to 0.95. Other options used were -n 10 -d 0 -M 0 -T 4. The remaining non-redundant contigs were annotated with HMM-based Pfam entries (Pfam-A) using HMMER v3.3. Mapping of reads onto contigs was done with BOWTIE2 v2.3.5.1 together with SAMTOOLS v1.9 to generate, sort and index bam files for later analysis.

Contig enrichment score calculation. The enrichment score for each contig was calculated using the normalized mapped reads (reads per kb per million, RPKM) from selection and control as follows: enrichment score=RPKM_{(selection)}/RPKM_{(control)}. The mapped reads counts were generated with Multicov using BEDTOOLS v2.29.2. Contigs with higher enrichment score represent more mapped reads in selection library relative to control library, therefore, are more likely to be associated with modification. We considered contigs with an enrichment score greater or equal to 3 to be modified and the rest unmodified. The calculation was done individually for three independent experiments.

Fisher's exact test and correction. The information including the number and type of Pfams on each contig was obtained with hmmsearch in the annotation step. We then re-organized the data and counted the number of contigs containing each type of Pfam in control or selection group. To avoid redundant counting, Pfams occurred multiple times on the same contig was counted only once. Fisher's exact test was performed for each Pfam to identify if the count difference between the selection and control group is significant. Because large-scale multiple testing was conducted for each Pfam, we did the Bonferroni correction to adjust the p-value. Both tests were performed in python with SciPy or Statsmodels modules.

Phylogenetic analysis. For each Pfam of interest, the protein sequences from contigs containing the Pfam were aligned with MUSCLE v3.8.1551. The resulting aligned fasta files were subjected to construct phylogenetic trees using the maximum likelihood method in the phylogenetic analysis program RAxML v8.2.12. We chose the -f a option to do rapid bootstrap analysis and the -m PROTGAMMAAUTO model to automatically determine the best protein substitution model to be used for the dataset. The parsimony trees were built with random seeds 1237. The online tool iTOL (https://itol.embl.de/) was used to visualize trees.

Co-occurrence network analysis. The presence-absence matrix with rows being the Pfams and columns being the contigs was generated with annotation output file from the previous step. We specifically performed co-occurrence analysis in the R package coocur v1.3 for the top 20 Pfams associated with modified contigs. Significant positive correlations (p-value <0.05) were exported and the network was visualized in Cytoscape v3.8.0 with prefuse force directed layout.

Differential conservation score. Protein sequences were assigned to two groups according to whether they were encoded on modified or unmodified DNA. After multiple sequence alignment, positions that have less than 50% residues present were ignored. Differential conservation score was calculated at each aligned position. For each position in the alignment, intra-group similarity scores were calculated by the average of all possible “within-group” pairwise similarities, while the inter-group similarity score was calculated from all possible “across-group” pairwise similarities using the BLOSUM80 matrix. For a given multiple sequence alignment column, let N₁ and N₂ be the number of residues for the modified and unmodified groups, respectively, the two intra-group similarity scores (I_{modified} and I_{unmodified}) were defined as

$$\\ {I}_{modified} = {\sum }_{i = 1}^{{N}_{1}} {\sum }_{j > i}^{{N}_{1}} M ( {a}_{i} , {a}_{j} ) \times \frac{2}{{N}_{1} ( {N}_{1} - 1 )}$$

$$\\ {I}_{unmodified} = {\sum }_{i = 1}^{{N}_{2}} {\sum }_{j > i}^{{N}_{2}} M ( {a}_{i} , {a}_{j} ) \times \frac{2}{{N}_{2} ( {N}_{2} - 1 )}$$

where M (aᵢ, aⱼ) is the value of amino acid pair aᵢ and aⱼ in the BLOSUM80 matrix. The inter-group similarity score (J) was defined as

$$\\ J = {\sum }_{i = 1}^{{N}_{1}} {\sum }_{j = 1}^{{N}_{2}} M ( {a}_{i} , {a}_{j} ) \times \frac{1}{{N}_{1} {N}_{2}}$$

The differential conservation score (S) was defined as the average of two intra-group similarity scores subtracted by the inter-group similarity score.

$$\\ S = \frac{{I}_{modified} + {I}_{unmodified}}{2} - J$$

Expression and purification of CT. The CT sequence was extracted from de novo assembled contigs. The expression plasmid was synthesized from GenScript (Piscataway, NJ). Two 6× His-tags were co-expressed at both the N-terminus and the C-terminus of the recombinant protein using T7 Express Competent E. coli (New England Biolabs, Ipswich, MA). Cells were cultured in LB media until an OD600 of 0.6 and induced with 0.4 mM IPTG (Growcells, Irvine, CA) for protein expression. One μM Iron (II) was also added to facilitate folding. The induced cultures were maintained at 16° C. in a shaker at 200 rpm for 23 hours. Cells were harvested by spinning down cell pellets at 3500 rpm at 4° C. for 30 minutes. Cell pellets from 4 L culture were resuspended in 160 mL buffer A containing 20 mM Tris pH 7.5, 500 mM NaCl, 0.05% Tween-20, 20 mM imidazole and sonicated using a Misonix® S-4000 Sonicator (Misonix, Farmingdale, NY) with 20 seconds on and 20 seconds off cycles until an OD260 plateau was reached. Cell lysates were spinned down at 13,000 rpm for 30 minutes in a pre-chilled centrifuge at 4° C. The supernatant was separated and combined with 0.2 mM PMSF(Sigma #78830). 50 mL of supernatant was loaded on AKTA™ (GE Healthcare, Chicago, IL) with 1 mL HisTrap™ column (GE Healthcare, Chicago, IL) pre-equilibrated with buffer A. The column was washed with 50 mL buffer A and eluted with a gradient of buffer B containing 20 mM Tris pH 7.5, 500 mM NaCl, 0.05% Tween-20, and 500 mM imidazole. Aliquots containing concentrated proteins were pooled and diluted 1:1 with 20 mM Tris pH 7.5, 5% glycerol and 0.05% Tween-20. The diluent was reloaded on AKTA with 5 mL HisTrap Q HP column, followed by a wash with 35 mL buffer containing 20 mM Tris pH 7.5, 100 mM NaCl, 5% glycerol, and 0.05% Tween-20 and eluted with gradient of a buffer containing 20 mM Tris pH 7.5, 1 M NaCl, 5% glycerol, and 0.05% Tween-20. Finally, collected fractions with concentrated proteins were pooled and mixed with equal volume glycerol for storage at -20° C.

CT enzyme assay. For enzyme assay using T4gt genomic DNA as substrate, 10 min incubation at 95 ° C.was performed to denature double stranded DNA. Then 0.38 nM denatured DNA was used for each 50 μL reaction with 1× NEBuffer 2.1 (New England Biolabs, Ipswich, MA), freshly prepared 10 uM Iron(II) sulfate hexahydrate (Sigma-Aldrich, St. Louis, MO), freshly prepared 10 mM carbamoyl phosphate and 5 mM ATP. CT was added to the reaction at 7.2 μM. The reaction mixture was incubated at 30° C. for 3 hours before adding 2 μL Proteinase K to inactivate the enzyme. After 30 minutes incubation at 37° C. with Proteinase K, DNA was purified with Zymo Oligo Clean & Concentrator Kit. For assays with synthesized single-stranded DNA oligos containing 5-hmdC, the heat-denaturing step was omitted. Oligos were added at 1.6 μM per 50 μl reaction with the same concentration of CT and other components added as listed before. Purification was performed using Oligo Clean-up and Concentrator Kit (Norgen Biotek, Ontario, Canada). For assays with free nucleotides, 0.5 mM of the corresponding nucleotide was used per reaction. For assays with synthesized RNA oligos, 1.57 μM RNA was added per reaction.

LC-MS and fragmentation analysis. Genomic DNA and synthetic oligonucleotides were digested to nucleosides by treatment with the Nucleoside Digestion Mix (New England Biolabs, Ipswich, MA) at 37° C. for 3 hours. The resulting nucleoside mixtures were directly analyzed by reversed-phase LC/MS or LC-MS/MS without further purification Nucleoside and Nucleotide analyses were performed on an LC/MS System 1200 Series instrument (Agilent Technologies, Santa Clara, CA) equipped with a G1315D diode array detector and a 6120 Single Quadrupole Mass Detector operating in positive (+ESI) and negative (-ESI) electrospray ionization modes. LC was carried out on a Atlantis T3 Column (Waters Corporation, Milford, MA)(4.6 mm×150 mm, 3 μm) at a flow rate of 0.5 mL/min with a gradient mobile phase consisting of 10 mM aqueous ammonium acetate (pH 4.5) and methanol. MS data acquisition was recorded in total ion chromatogram (TIC) mode. LC-MS/MS was performed on an Agilent 1290 UHPLC (Agilent Technologies, Santa Clara, CA) equipped with a G4212A diode array detector and a 6490A triple quadrupole mass detector operating in the positive electrospray ionization mode (+ESI). UHPLC was performed on a XSelect® HSS T3 XP column (Waters Corporation, Milford, MA) (2.1×100 mm, 2.5 μm particle size) at a flow rate of 0.6 mL/min with a binary with a gradient mobile phase consisting of 10 mM aqueous ammonium formate (pH 4.4) and methanol. MS/MS fragmentation spectra were obtained by collision-induced dissociation (CID) in the positive product ion mode with the following parameters: gas temperature 230° C., gas flow 13 L/min, nebulizer 40 psi, sheath gas temperature 400 ° C., sheath gas flow 12 L/min, capillary voltage 3 kV, nozzle voltage 0 kV, and collision energy 5-65 V.

Sequence preference of CT. Library preparation was performed as described above. For each library, 1 μg genomic DNA mixture (Lambda:XP12:T4gt=1:1:1 by molarity) was used. Libraries were subjected to CT treatment as described. Purified DNA samples were heated at 90° C. with formamide to generate single-stranded fragments before the deamination reaction. One μL APOBEC was added per reaction to both CT-treated or control (untreated) samples. The reaction mixture was incubated at 37° C. overnight. Samples were purified using Zymo Clean & Concentrator Kit and pair-end sequenced (75 bp x2) with Illumina MiSeq® (Illumina, San Diego, CA). Raw reads were trimmed with TrimGalore. Methylation was analyzed with Bismark v0.22.3 and plotted with RStudio v3.6.3.

Synthesis of 5-hmC RNA oligonucleotide. Forward and reverse DNA templates were annealed at 95° C. for 4 minutes and slowly cooled for 20 minutes. RNA synthesis was performed with HiScribe™ T7 High Yield RNA Synthesis Kit (New England Biolabs, Ipswich, MA). One ug of annealed DNA template was used per reaction with 1.5 μL T7 RNA Polymerase Mix. 5-hydroxymethylated triphosphate (5-hmCTP) was used with the other three nucleotides ATP, UTP and GTP at 7.5 mM each. The reaction was incubated at 37° C. for 4 hours. Two ul Nuclease-free DNase I were added to each reaction to digest DNA templates, followed by incubation at 37° C. for 15 minutes. Synthesized RNA was purified with Norgen Biotek Oligo and Concentrator kit and stored at −80° C.

Nucleotides and synthesized oligos. Single-stranded DNA oligos used in enzymatic assays were purchased from IDT. The sequences are as follows:

5-hmdC-1:
(SEQ ID NO: 24)
5'-TGTCCGATAGACT{5-hmdC}TACGCA;
5-hmdC-2:
(SEQ ID NO: 25)
5'-AACTCGCCGAGGATTT{5-hmdC}TAC;
5-hmdC-3:
(SEQ ID NO: 26)
5'-{Fam-AmC6}ACACCCATCACATTTACAC{5-hmdC}
GGGAAAGAGTTGAATGTAGAGTTGG.

The DNA templates for synthesizing RNA were purchased from IDT as follows (T7 promoter sequence was underlined):

Forward:
(SEQ ID NO: 27)
5'-GACCTAATACGACTCACTATAGGGAGTGA
GAAGATGGTCTAGGTGTTTATTGGTGATGAA;
ComRev:
(SEQ ID NO: 28)
5'-TTCATCACCAATAAACACCTAGACCATC
TTCTCACTCCCTATAGTGAGTCGTATTAGGTC.

5-hmdCTP (D1045) and 5mdCTP (D1035) were purchased from Zymo Research (Irvine, CA). 5-hmdUTP (N-2059) and 5-hmCTP (N-1087) were purchased from Trilink Biotechnologies (San Diego, CA). Code availability. Custom-built bioinformatics pipelines are available at https://github.com/linyc74/Meta GPA.

### Example 2: Metagenomic Analysis of a Human Microbiome from Sewage (Meta GPA)

The phage fraction of the microbiomes was obtained to increase the prospect of finding novel base modifications in particular, modified cytosines. An enzymatic selection was carried out too distinguish between known and unknown forms of DNA modification and DNA containing unmodified cytosine was removed. Enzymatic selection consists in a three-step treatment of the library as illustrated in FIG. 2A. The first and second steps were analogous to the EM-seq protocol that identify methylated cytosines. The third step utilized Uracil-Specific Excision Reagent (USER) that recognized and fragmented DNA containing uracil so that these are depleted from the library so that the remaining DNA contained mostly modified cytosines. Using the premise that many forms of cytosine modification, including those unknown to date, were naturally protected from deamination by APOBEC, the selection method described herein was designed to enrich for such nucleic acid modifications.

Genomic DNA from E. coli (containing unmodified cytosine, dC) and T4gT phage (containing 5-hmdC which fully replaced dC) were sheared and libraries formed and assayed in order to determine whether modified DNA resulted from phage encoded modifying enzymes could be detected. Samples were split into two groups with or without enzymatic selection respectively and quantification of DNA was performed using qPCR. Substantially, complete removal of DNA containing unmodified cytosine resulted in less than 0.5% recovery of unmodified DNA. Conversely, 40-50% of library DNA was recovered with modified cytosine following the same treatment. To test the sensitivity and efficiency of this method, we serially diluted modified DNA with spiked in unmodified DNA at 1:3, 1:10, 1:100 and 1:1000 molar ratio and carried out the enzymatic selection. Recovery rates were calculated and compared to no-enzyme treatment control. Even at 1:1000 level, an average of 48.6% modified DNA was retained relative to no-enzyme control. This result showed the capability of present methods to concentrate trace amounts (picogram-level) of modified DNA from a complex sample.

The phage fraction of each sample was precipitated with polyethylene glycol (PEG) followed by DNA extraction using phenol/chloroform (see Materials and Methods). Sheared DNA was ligated to Y-shaped adaptors containing pyrrolo-dC (to protect adaptors from enzymatic degradation). Library pairs were subjected to either enzymatic selection or control (FIG. 3A). Additionally, spiked-in genomic DNA mixture of E. coli, XP12 (containing modified 5-mC, 5-mdC, which fully replaced dC) and T4gt were added to each sample after library preparation. Recovery of spiked-in modified DNAs was detected as expected (FIG. 3A). We observed consistency of k-mer composition between replicates, demonstrating that our enzymatic selection for modified DNA is reliable and the data is reproducible (FIG. 3B). Normalized k-mer frequency plots showed diversity of k-mer composition from different sources/samples, while highlighting a small portion of k-mers that were either specific or highly enriched in the selection libraries (FIG. 3B). To translate and study the biological entities from the dataset, we separately assembled the sequencing reads from the selected and control datasets from the three samples into contigs and removed contigs that were either too short (less than 1000 bp) or redundant (Methods). Then, the ratio between the normalized coverage in the selection library (RPKM_{(selection)}) and the normalized coverage in the control library (RPKM_{(control)}) defines the enrichment score for each contig (Methods). A high enrichment score (>=3) suggests that the contig is derived from DNA containing modified cytosine (modified contig). In total, about 4000 modified contigs were identified from three DNA samples.

To study the functional units coded in each contig, annotations using Pfam protein families database were performed. For each Pfam domain present, we conducted Fisher's exact test, and corrected the p-value to identify the subset of Pfam domains that were significantly associated with modified contigs. Interestingly, there was a high degree of overlap of the top associated Pfams among different samples, suggesting that a group of universal protein families for DNA modification may exist. The results from these individual DNA samples were consistent. As a result, the three datasets were pooled to achieve higher statistical power. The resulting top associations (see FIG. 4A) contained a number of Pfam domains found in enzymes involved in DNA synthesis/modification, for example thymidylate synthase homologs (PF00303.20) producing hydroxymethylpyrimidines, DNA ligase (PF14743.7, PF01068.22), and cytidine and deoxycytidylate deaminase zinc-binding region (PF00383.24) (FIG. 4A). Meanwhile, our analysis demonstrated a group of Pfams that were not previously known for a function in DNA modification and thus may be novel DNA modifying enzymes or critical regulators. To refine the Pfam domain candidates, we conducted phylogenetic analysis for each Pfam significantly associated with modified contigs. Towards this end, all instances of a particular Pfam domain were aligned and a maximum likelihood model was used to associate phylogenetic relatedness with the status of the contig of origin (modified/unmodified) (see for example, FIGS. 4B-4D). Particularly, several Pfams, including CT N-terminus (PF02543.16) and C-terminus (PF16861.6), exhibited a clustered pattern in which sequences from modified were clustered separately from unmodified contigs (FIG. 4B). This clustering pattern of modified contigs restated the association of the Pfam-of-interest with a potential differentiated phenotype of modification. Moreover, this can serve as evidence for refined taxonomy and may suggest a subfamily with specific functions.

We extended the analysis to study co-occurrence of Pfam domains associated with modification (Methods). Surprisingly, we found several mutually correlated Pfams (FIG. 4C). For example, the most frequently co-occurring Pfams with CT C-terminus (PF16861.6) were CT N-terminus (PF02543.16), thymidylate synthase (PF00303.20), phosphoribosyl-ATP pyrophosphohydrolase (PF01503.18), dCMP deaminase Zn-binding region (PF00383.24), and MafB19-like deaminase (PF14437.7) (FIG. 4C). Congruously, thymidylate synthase also co-occurred with CT N-terminus, phosphoribosyl-ATP pyrophosphohydrolase, dCMP deaminase Zn-binding region, and MafB19-like deaminase. These co-occurrences were found to be specific to modified contigs. For example, the CT N and C terminal domains were found in the same genomic context as the thymidylate synthase genes only in the modified contigs (FIG. 4C). In the unmodified contigs, CT N and C terminal domains were flanked by genes with unrelated functions such as glycosyltransferases group 1 or tRNA N6-adenosine threonylcarbamoyltransferase domains.

The CT open reading frame was cloned from a modified contig originally sequenced in sewage #2 containing both the thymidylate synthase and CT sequences into pET28b vector, expressed and purified the 63 kDa enzyme product. The predicted reaction was tested by enzymatic assays and results showed that each component, namely carbamoyl phosphate, ATP, 5-hmdC from genomic T4gT DNA and the enzyme, was indispensable for the reaction. The expected product was detected by liquid chromatography-mass spectrometry (LC-MS) and confirmed with corresponding fragmentation patterns (see for example, FIGS. 7B and 7C). Nearly 70% of 5-hmdC were converted into 5-cmdC in the denatured T4gt genomic DNA. Interestingly, our CT was active only on denatured single-stranded, but not double stranded DNA. When using synthesized single-stranded DNA oligo containing an internal 5-hmdC site as substrate, the conversion rate was nearly 100%. CT was tested to determine if it could react with free deoxynucleoside triphosphate. LC-MS results demonstrated about 60% conversion of 5-hmdCTP. No activity was shown for 5mdCTP or 5-hmdUTP, indicating the CT is specific to 5-hmdCTP and the reaction could take place before the nucleotide is incorporated into DNA (FIGS. 7BB-7C).

NEBs3
(SEQ ID NO: 1)
MSDLLLTLGHNASAIAISVGDDGAAKVENAYELERLTGKK
SDSAFPIDAIIALKERGMDKIDRVYVSHWSPTGRVDDLKA
KYWDRSIFPPHVPVITQESMNLTHHDCHAQAAMAFAGSSF
PTKDTGVLVVDGFGNLAEHLSYYRVQAGGQLHLMRRWYGY
GTSLGLMYQYATSFLGLKMHEDEYKLLGYGARVATIGCDM
DVLNQRIFTEAQAFLKRFRSLNSFEMSPDLAGLPAVQEKW
AERFAAILDDVGFKGSSSTYEARCIVGYAVQQLLEIVIRN
LFMADLPKPTNLIVTGGVAFNVELNRMLLGLIPGKLCVMP
LAGDQGNALGLWAFSNRRAKLDFGDLCWGRREMTLGEPGP
DTPLPDGMIVVEHDTPAVYEAIAEQLKTVGFINIVRGNME
FGPRALCNTTTLARADDRAVVEEINRINGRDTVMPFAPVV
SAHEWLRYFPDASRLHRSAEFMICAVQYAPGLGEQVPGAA
LRTVKGLYTGRPQVYSSKYEWDSVTRILDDYGLLINTSFN
VHGVPICLDLKHVVHSHQFQRERNPNVRTIVIAN*
SEQ ID NO: 2
atgagcgacctgctgctgaccctgggtcacaacgcgagcg
cgatcgcgattagcgttggtgacgatggcgcggcgaaggt
ggagaacgcgtacgagctggaacgtctgaccggcaagaaa
agcgacagcgcgttcccgatcgatgcgatcattgcgctga
aggaacgtggtatggacaaaattgatcgtgtgtacgttag
ccattggagcccgaccggtcgtgttgacgatctgaaggcg
aaatattgggaccgtagcatctttccgccgcacgtgccgg
ttattacccaggagagcatgaacctgacccaccacgattg
ccatgcgcaagcggcgatggcgtttgcgggtagcagcttt
ccgaccaaagacaccggcgttctggtggttgatggtttcg
gcaacctggcggaacacctgagctactatcgtgtgcaggc
gggtggccaactgcacctgatgcgtcgttggtacggttat
ggcaccagcctgggtctgatgtaccagtatgcgaccagct
ttctgggcctgaagatgcacgaggacgaatacaaactgct
gggttatggcgcgcgtgttgcgaccatcggttgcgacatg
gatgtgctgaaccagcgtattttcaccgaggcgcaagcgt
tcctgaagcgttttcgtagcctgaacagctttgaaatgag
cccggatctggcgggcctgccggcggttcaagagaagtgg
gcggaacgtttcgcggcgatcctggacgatgtgggtttta
aaggtagcagcagcacctacgaggcgcgttgcattgtggg
ttatgcggttcagcaactgctggaaatcgttattcgtaac
ctgttcatggcggacctgccgaaaccgaccaacctgatcg
tgaccggtggcgttgcgtttaacgtggagctgaaccgtat
gctgctgggtctgattccgggcaagctgtgcgtgatgccg
ctggcgggtgatcagggtaacgcgctgggtctgtgggcgt
tcagcaaccgtcgtgcgaaactggactttggtgatctgtg
ctggggccgtcgtgagatgaccctgggtgaaccgggtccg
gacaccccgctgccggatggtatgatcgtggttgagcacg
acaccccggcggtgtatgaggcgattgcggaacaactgaa
gaccgttggcttcatcaacattgtgcgtggtaacatggaa
tttggtccgcgtgcgctgtgcaacaccaccaccctggcgc
gtgcggatgatcgtgcggtggttgaggaaatcaaccgtat
taacggtcgtgacaccgttatgccgtttgcgccggtggtt
agcgcgcacgaatggctgcgttactttccggatgcgagcc
gtctgcaccgtagcgcggagtttatgatctgcgcggtgca
gtatgcgccgggtctgggtgaacaagtgccgggtgcggcg
ctgcgtaccgtgaagggtctgtacaccggccgtccgcagg
tttacagcagcaaatatgagtgggacagcgtgacccgtat
cctggacgattacggtctgctgattaacaccagcttcaac
gtgcacggcgttccgatctgcctggatctgaaacacgtgg
ttcacagccaccagtttcaacgtgaacgtaacccgaacgt
tcgtaccatcgtgattgcgaactaa
NEBs1
(SEQ ID NO: 29)
MKTALALTLGHNSSAVLIQDGEIIAGYEQERFSAKKSDSA
FPKDAIKELALRYDLPSNTTVCVGHWFLDHQLPPVANKYW
DPDHIREMFPDGEVLSLDAQFSHHDSHLEAAMVFAGKNFA
PSYHALVLDGFGSSGECISVYAVNKGSYRLLERWFGFDKS
LGMLYQYATAFLGMKMHNHEYKMLAYEVHLHALNYDVKRL
DELVAKCADEWLTKMFRHQIHPGTDPLISLEALPNIQNTI
EQMLGDVLIELDAGDVDIHDKRCIISYFVQHVVESVVLSL
AKVYSPENLLLVGGLFYNVKLNNLLASKVSGRTCIMPIAG
DQGAGIGVYQRYFGDLKWPEHLFWGYRNLDFQSDDESMVI
VDTMEDALPLLAAELESVGFVNLVRGAMEYGPRALCNTTT
LAIPDRDVGATINRINDRTNEMPFALFMSKSQADDLFVDC
DKVYKSLEYMICTRNFKPGKAESCLGGAHYYPDLDVYTCR
PQITDDEHLLVLLDKFGPLINTSYNYHGVPIVLGEEQIKH
THKMQRQTAPDVAFKTIIVRK*
Modified_002
(SEQ ID NO: 30)
VQPKLNGLNNGISGMKALLLTLGHNSSAILIEDNKVVWGY
ETERISGLKSDSRFPMPFIEKYLGKRPEFDVVYVTHWATD
GVLSSMSAKHWDPSYFDGKPIRTLSIDRSHHDTHMSAAMC
YAGKEFFKHKHSVHGLIIDGFGTLGEHLSIYDLSDGTPKL
IKRIHGYETSLGLWYQYATAFMGMKMHEDEYKLLGYETHC
HGPIGDQLIHEADLYALEWIDKLGKSIYKSIYDPMYSLDA
LGAVKTNVFKHLAKVCRQYNLADPTESVARSILALYVQRV
LESVVLNLIGHYNMKNVILSGGCFYNVKLNKRIIDEIDGQ
VCIYPLAGDQGNALGLYAMDHPEFIFPDNLNWGIRKLKNV
GEIRGIFYADSYDEATEMITSSLAHHGYVNLVRGAMEEGP
RAMCNTSTLAIPTMDVVQSINTMNNRNTVMPMAPVMTEYM
YKTMFERTSQVWRSQHHMITALEYVEYPPSHLLGIAHEYL
MPHHHHTGRPQVTHDPFMVKILDHIGHPLINTSFNYHGMP
IALGMEEVISNHRMQQQREPSISTVVITNA*
Modified_003
(SEQ ID NO: 31)
IKSNSSFPIKAICRLNDLYHLPSDVKVYVSHWFLDGTLEH
ANAKHWSIRLLKTLFPYASIRGINPDKCTHHAAHALSAEV
FAGPDFPKDHHVLVVDGFGTQGECYSAYAYDSDISGLRVG
LAYHGFDRSIGLFYQYATAYCGMKMHQHEYKMLAYETHLT
EIMPCPYKVNELNEYIDLYSDNYDFVVSTYEVSLDRLSSV
QAIINCVLDAYLKYVKRNRLTLTDPYGDRSKKILVSYFTQ
RHTENTVRKFVKSIKPTNLIVAGGVFFNVKLNKMICDMIP
GKFCAMPLAGDQGAGLGVYQYYNGDLIWPDHLFWGHRDLV
LDLPKGLMRKGTVEDVKNCLFSNRLVNVVRGSMEFGPRAL
CHTSTLGFPAKDVAERINRMNDRTNEMPFALVVTRDQADE
LFEDIDKVHKSLEYMIVTRNFKKGREKKYLGGAHYYPLTK
EYTCRPQITDDPFMIEILNEFGPLINTSFNYHGCPIVFDQ
NHISHSHFMESKHELISTIIIS*
Modified_004
(SEQ ID NO: 32)
MSSSIFINSAFSIISCFISSMTILSILINSAIIIHSILLY
IISCFLVENTFFDKLNTMNTLGISCGSHDAAIAAIKNNEI
VFASQSERYSKNKNDLYLNRDVILDALYSLDYNVDSLCFY
ENILNKKTRQFFAGQYYNVFVDKPLSSWYNNIKNIYTNYK
LLSKLKIHQIDHHKSHAALAFASPYNKAIVLVIDAIGEWD
TISIWLYNNSLNRKLIKLESYSYPQSLGLFYTAFTDYVGL
KANEEEYIFMGMAAFGKPIYKDMIRKYLFNSKGNIKINLH
TGIPKDLLNININDFKTKENIASSVQDLTEEYIFKLLNKI
KYYQDIYKTKNLIYSGGIALNCVANSKIQKEFSNMWIFPN
PGDSGSSLGCALAYLNNKTKFDSVYLGYNICYNSICPNDI
SEYLLNNKICGIALGKAEFGPRALGNRSLLADPRGNEIKD
KVNEIKKRDKFRPFAPIIMEEYFKEYFEVPNNIMNSSYMQ
YVYKCKYPDKFPAIVHIDGTSRVQTINEKQNAFVYNVLKS
FYNKTGCPMLLNTSLNIKG
Modified_005
(SEQ ID NO: 33)
MAESVERQEQEMKALLLTLGHNSSAVMIENDQIVWGYETE
RLSGLKSDSRFPMPWIEKLPNTDLRNIDIVYVTHWAPDGQ
LSSMSRKHWDPFYFDGVPIRTLSADRTHHDTHMAAADCYA
GPAFTKHRKPGTFGFVVDGFGTMGEHFSVYEYQSDHTPKL
VRRVHGYGTSLGLWYQYATSFMGLKMHEDEYKLLGYEVHC
PLNLINDIDMAAAVKADEWIDEMKKSIYGSKYDPLYDLRA
LENVKNKIFEHLNQKCKDFGVTDPTSHEGRCVLAYYVQAV
LEGVVTTLIHQFKPKHVVLSGGCFYNVKLNKSIVDRIEGQ
TCVYPLAGDQGNALGLYFMDNPQFCMPNHFNWGKRDLRDV
GEVKGLVCVKDTNSALKQLTDRLYQVGHVNLVRGSMEFGP
RAMCNTSTLALPTAASVELINAMNDRNTVMPMAPVMTMAM
YRSLFERWDRVWGSYRHMIVAMEYVEHPGDELRGVAHEYQ
VPYHHHTGRPQVVPDTDWLMGALLARFDGPLINTSFNFHG
QPIAFNMESVIKNHMLQYQRDPSTFTVVITNE*
Modified_008
(SEQ ID NO: 34)
MKSLLLTLGHNSSAIMVEDGKIKWGYETERISRQKSDSHF
PEAVLSTMDVKQVDEVYVTHWSPDGRLSSMSAKHWHPARF
ENTPIRTLSVGLSHHDTHMHGAMCYAGNSFPYSAGTYGLV
VDGFGTLGENLSVYNLTSGKPILKKRVHGYGTSMGLWYQY
ATAFMSMKMHEDEYKILGYEAHVPEDLVAKLNAAADVRAS
YILDEMHKSVYGSAYDPMYSLEALANIKNEIFTHLYNVCT
QFSVTDPSSQSGRIIIGYYVQAVLESVILTVVRSLNARNL
ILSGGVFYNVKLNKRIIDECEGLVCVYPLAGDQGNAIGLY
YLDHPEFEFPSNLNWGHRTLRDPGNIPNMIFARSEEEAGM
LIHRELAQGIGYINLVRGNMEFGPRAMCNTSTIAMPTRRC
VQAINAANNRNTFMPMAPVMTRECYRQLFENTD
Modified_010
(SEQ ID NO: 35
LDAQFSHHDSHLEAAMVFAGKNFAPSYHALVLDGFGSSGE
CISVYAVNKGSYRLLERWFGFDKSLGMLYQYATAFLGMKM
HNHEYKMLAYEVHLHALNYDVKRLDELVAKCADEWLTKMF
RHQIHPGTDPLISLEALPNIQNTIEQMLGDVLIELDAGDV
DIHDKRCIISYFVQHVVESVVLSLAKVYSPENLLLVGGLF
YNVKLNNLLASKVSGRTCIMPIAGDQGAGIGVYQRYFGDL
KWPEHLFWGYRNLDFQSDDESMVIVDTMEDALPLLAAELE
SVGFVNLVRGAMEYGPRALCNTTTLAIPDRDVGATINRIN
DRTNEMPFALFMSKSQADDLFVDCDKVYKSLEYMICTRNF
KPGKAESCLGGAHYYPYLDVYTCRPQITDDEHLLVLLDKF
GPLINTSYNYHGVPIVLGEEQIKHTHKMQRQTAPDVAFKT
IIVRK*
Modified_006
(SEQ ID NO: 36
TLGHNSSAIFVEDGQVICGYEEERLSEIKSDSAFPKKAID
LITKHYCNRFDNVFVGHWFNNGNLTDCKYWDEKYILQFVK
DPRCIKSLNVDFTHHDSHRVAAECFYKNYSQQLLDENDYC
IVADGFGTFGESLSIYGYHNGKAHVKSRVFDYINSLGLLY
QYATLYLGMKMHNHEYKMLGYEAHIHEHFTQDEIFIMDGW
ADLQTDKILKSMNSVSLKQTDPIVNTDALDFVRSSIFDLL
DTFCDKFDIKSVENDVIEKKRVGVSYYVQKIVETVILDIV
ASLGKVKTLLLSGGLFYNVKLNNKISKLVGNLCILPVAGD
QGAGLGVYHHYVGDLQFPRSLCIGHRFLDADDFKDIDGIE
TFENADEMYHRIEECLADNGFVNVVKSSMEFGPRALGSTS
TIAYPEVDVVKIINELNDRTFVMPMAPMMTQEQFNMLATS
GFNVIGSNEFMVSTVDVHKYFIEGIEGAVHRYVDAATCRP
QIVDGNPFYKTLCSNLGPLINTSFNYHGVPIVFDSKQIIH
SHQCQQKNANGKYNVITLIYTGSN*
Modified_007
(SEQ ID NO: 37
MKSLVLTLGHNSSAIVIEDGHILGGYEEERLSGIKSDSSF
PIKAICRLNDLYHLPSDVKVYVSHWFLDGTLEHANAKHWS
IRLLKTLFPYASIRGINPDKCTHHAAHALSAEVFAGPDFP
KDHHVLVVDGFGTQGECYSAYAYDSDISGLRVGLAYHGFD
RSIGLFYQYATAYCGMKMHQHEYKMLAYETHLTEIMPCPY
KVNELNEYIDLYSDNYDFVVSTYEVSLDRLSSVQAIINCV
LDAYLKYVKRNRLTLTDPYGDRSKKILVSYFTQRHTENTV
RKFVKSIKPTNLIVAGGVFFNVKLNKMICDMIPGKFCAMP
LAGDQGAGLGVYQYYNGDLIWPDHLFWGHRDLVLDLPKGL
MRKGTVEDVKNCLFSNRLVNVVRGSMEFGPRALCHTSTLG
FPAKDVAERINRMNDRTNEMPFALVVTRDQADELFEDIDK
VHKSLEYMIVTRNFKKGREKKYLGGAHYYPLTKEYTCRPQ
ITDDPFMIEILNEFGPLINTSFNYHGCPIVFDQNHISHSH
FMESKHELISTIIIS*
Modified_n_018
(SEQ ID NO: 38
LKFMKENNMKLLLTLGHNSSAILVDDQKVLCGYEEERLSG
VKSDSSFPSLSIKKILEFYPQAKMEVDEVCVSHWFWSWDL
VENKYYQPKFIAANFPKASVKSVSYYETHHDLHAKSLWNF
LDGDNSGLTVVADGFGNFGECLSIYIDGKLYHRSYEVSRS
LGIMYQYAIRYLGMKENQDEYKLLGYEQNVSEAFKNNITE
DIIEVSNVMYKSLTNKVAMKKDMLAELDKAWNYWRVTFSE
IDDSEQDRAKIAYFVQQILENVMIKIIRSFNIKNIKVTGG
VFYNVKLNNLILRYADKFEAYPLAGDQGCALGFT
Modified_014
(SEQ ID NO: 39
LKFMKENNMKLLLTLGHNSSAILVDDQKVLCGYEEERLSG
VKSDSSFPSLSIKKILEFYPQAKMEVDEVCVSHWFWSWDL
VENKYYQPKFIAANFPKASVKSVSYYETHHDLHAKSLWNF
LDGDNSGLTVVADGFGNFGECLSIYIDGKLYHRSYEVSRS
LGIMYQYAIRYLGMKENQDEYKLLGYEQNVSEAFKNNITE
DIIEVSNVMYKSLTNKVAMKKDMLAELDKAWNYWRVTFSE
IDDSEQDRAKIAYFVQQILENVMIKIIRSFNIKNIKVTGG
VFYNVKLNNLILRYADKFEAYPLAGDQGCALGFTNVKYDN
LFWGKRSVGNIDVNEVIDNIKTYGYSELFHGDMEFGPRAL
CNTTCLAVPTSEMVEKINAQNGRDTVMPMAPVVTEKFMNK
WFNDIEKVGKSKNFMIIAYDYK
Modified_n_019
(SEQ ID NO: 40
QKFIKKEIKMKLLLTLGHNSSAILVEGEKVVCGYEEERLS
GVKSDSSFPSLSIKKILEFYPQAKLLVEEVCISHWFWSWD
LVENKYYQPKFIKANFPKAKVTSVDFNNTHHDLHARSLWN
FLDGDQSGLTIVADGFGNFGECLSVYVDGILKHRSYEVAK
SLGIMYQYAIRYLGMKENQDEYKLLGYEQNVSEAFKKQIA
GSITEVSSHLYKELSCVQNGMKKDMLAELDKAWNYWKVTF
SEVDDADQDRAKIAYFTQQVLEQVMMAIIQDYDIKNIKVS
GGVLLQKIHLQKYICLLFLLVLFLL
Modified_013
(SEQ ID NO: 41)
GGFTMNLCLSLGHNSSAVLVDDYGRIIIGYENERLSGIKS
DSHFPQDAIKEIGKYYYLSLIDRVYVSHWATFGSVEEMSA
KHWRPDILSELCPNAILQRDVDHHECHVSALRAFTSETFN
WEIVADGFGNFNETMSIYHKGALIHRCFGYEKSLGLFYQY
ATAYMGLKMNQDEFKLLGYESKIKEVVSNKCIVEILSVAQ
KTADKFFRSIIDTSLEPKYDAVAGLEALPNLRVKIENNLD
NLLYVKLKDVYSASRGSDSDRVVIAFYVQSVIESVLRKVV
LHFGMDEVALTGGIFMNVKLNNIISKLVNKISVMPICGDQ
SGGLGAYEYYNGNLQWPDHLFWSDRGGLFTLDDGDPDMVV
FDEEVDALRYISLCLNADRIVNLVQGKGEFGARALGNTST
LALPTSNNVEYINHLNQRSTIMPMAGMISPKALSNYTDAD
KVHKSLEYMIITLDAKKVDSSTEGCHHNHPINGSITNRVQ
LVDNGSLVGEIVKRFDCLINTSFNVHGVPIVLTSAQVQKS
HDSQKSLDYFDRMVTVIVRN
Modified_015
(SEQ ID NO: 42)
LGIMYQYAIRYLGMKENQDEYKLLGYEQNVSEEFKEDIQD
MIEENVALSYSFLKTKMPMKQDMLAELDKAWNYWRVTFSE
VDNAEQDRPKIAYFTQQVLEKVMMKFIESYSISNIKVTGG
VFYNVKLNNKILKYASKFEAHPLAGDQGCALGFIPVRYDN
LFWGIRFVSEGIDVARVIEDIEHQGYSELFHGDMEFGPRA
LCNTTCLALPTSEMVEKINAQNGRDTVMPMAPVVTEKFAK
TWFKDIDKVGKSKNFMIIAYDYKGMNENIKGAAHIDSDRD
VYTGRFQLATDPRIIKILNHFGGILINTSLNAHGQPIIYD
QNDFAMMRYIQDEVQKNR*
Modified_c_016
(SEQ ID NO: 43
FRPFAPAILEEFADSYFDGPMNEYMQYTSQAKHDYKSVTH
VDGSARVQVVKKNCRSILRPILEEFYNQTKCPMLLNTSLN
IKGQPMVNTIEHAKEFQNKYKVKVF*
Modified_n_020
(SEQ ID NO: 44
KMRYIGLHAYGHDANKTTTILENGVVVENKHIEYERLLRE
KRVKFDLIDKSTFLESEENSQINISISGHHNLQKIMGDED
FKIDYLPSINRKLDDEKYITHPILGNKTMLISHHLAHASY
AYYTRPYYMKDSDVFTYDGRGIFTDSTFHNNTKSITNDVI
GIGMLWDLIGRCINRDAFTEGKVMGLSAYGKYNSKLESIF
LQYIKEYTTKDNTDIAWFPTEEEHIKLLKEYSELLKPYDN
KNIAATLQRFSEVYVLEYLKRYKTSDNLCIGGGVGLNGYI
NQK
Modified_c_017
(SEQ ID NO: 45
IFGASVIFSNHSPTMKDYLNERVKHREWYRPFAPAILKEH
VTEWFENITDAPNMLKIARYKKGMGEKVPAVCHIDYTGRL
QTVTKESNEHFYNLISAFNGISGVPILLNTSFNDNGEPIV
DSPQDALKTFNNTNIDILVINNYVYEKE*
Modified_011
(SEQ ID NO: 46)
MKKALMITLGHNSSALFFDGENTVGYEQERLDKIKSSSAF
PKDAIEKIKEQYDIKDSYLFVSHWYDSFDLKTVDYSKHVD
NIFLEKLVNDFNLTYIPLSEDFTHHDAHAYSSLSFLLNFM
NKEKTKLLVGDKIHFLVVDGFGNRQEVVSLYSLNKKDLFV
KNNNLKKIASFGGYFKSLGLMYQNATSYCGMKENQDEYKF
LGYESHITSVLNSTDLLNLDTLLDDRIRDIFDNMFVEYRY
CVVESFINVGDLRLVKKYWYEIFDSILSNIGIKNDKNFVS
RVIIGYSIQRIIEDILLKIVKMYDIKNLCVSGGVFYNVKL
NNRLLNSVPGIFSAMPLAGDQGAAIGMYKKYVGDFYFKNM
CFGIRDEFDYSLLKQVDFKDKIFVTKNRHDVVEKIAELLK
QDNIVNFVEGNMEFGPRALCHTSTLSLPSSTNVDYINTLN
KRNTVMPMAPVMLQDFVHTFFDTKTYDRIIGSDMFMIVTL
NYKEFDEKYNGVSHKYPKLDVYSGRPQVVSSGDNKIVKNV
LENLSDYTKCLVNTSFNVHGNPILYDYKSIFEDFVFQCNQ
AVENNIKIPYLVLYV*
Modified_012
(SEQ ID NO: 47)
MEIIIMPKYSLAISLGHNSSAVLIEDGHILIGFENERISK
VKSDSAFPKLAILEIGKFYDLHSVEDIYVSHWEVTGNVDN
MSLKHWRKDFLLTACPQARIHADYDHHDCHVAALRTFSGG
NKWEIVADGFGNFNEVISIYKDGNLLHRVFGYEKSLGLLY
QYATAYLGLTMNQDEYKLLGYESKIGLTIDANRLSILQDE
IFNYADKYFKAIIKPKIVPKYDSVAGLDALVNIRLNITNH
LDYILYKKLGLVKSETSISMQRIIVAFYIQSVVEIVMEKL
VRAFDMKDVGLCGGLFMNVKLNNRISQCVKNMDIMPLCGD
CGAGIGAYQIHRGDLKWPDHLYWGHRNYFLKPVGKDLICY
LHFNEALQELITNLRKDNIVNFVCGSMEFGARALGHTTTF
ALPTVVNVHYINLLNERNTIMPMAGMMSDHCMRANYERYS
IAPKSVEYMITTLDLKLGSTLEAQGVHHLDVVRGVFTNRV
QVVGEGHDFYPLVEEFGTVINTSFNIHGTPIVYDADDVMK
AHSFQKARDRDNRVVTIVVVA*
>tobZ O-carbamoyltransferase_TobZ
(SEQ ID NO: 48)
HHHHHHMRVLGLNGWPRDFHDASAALLVDGRIAAFAEEER
LTRKKHGYNTAPVQAAAFCLAQAGLTVDDLDAVAFGWDLP
AMYRERLGGWPHSDSEALDILLPRDVFPRRTDPPLHFVQH
HLAHAASAYYFSGEDRGAVLIVDGQGEEECVTLAHAEGGK
ITVLDTVPGAWSLGFFYEHVSEYTGLGGDNPGKLMGLAAH
GTTVDETLSAFAFDSDGYRLNLIDPQARDPEDWDEYSVTE
RAWFAHLERIYRLPPNEFVRRYDPAKGRVVRDTRRDPYEY
RDLAATAQAALERAVFGLADSVLARTGERTLFVAGGVGLN
ATMNGKLLTRSTVDKMFVPPVASDIGVSLGAAAAVAVELG
DRIAPMGDTAAWGPEFSPDQVRAALDRTGLAYREPANLER
EVAALIASGKVVGWAQGRGEVGPRALGQRSLLGSAHSPTM
RDHINLRVKDREWWRPFAPSMLRSVSDQVLEVDADFPYMI
MTTKVRAAYAERLPSVVHEDWSTRPQTVTEASNPRYHRML
TELGDLVGDPVCLNTSFNDRGEPIVSSPADALLTFSRLPI
DALAVGPYLVTKDLRH
>YP_239307.1 putative cabamoyl transferase
[Xanthomonas phage Xp15]
(SEQ ID NO: 49)
MTYALAISLGHNSSAILIQDGVVLAGYEEERFSGVKSDSK
FPYQSILELKRRFDLPSDTDSFVGHWFLDAQLPAPNKYWD
PDFLRSHFPNGSIDSLNADFTHHDSHLASAMVFAGEEWSD
KSYTAVVADGFGSYGECLTIYAVTGQSYSVKHRVFGFEKS
LGMLYQYATAFMGMKMHNHEYKMLAYEVHIGEVLDVDQID
RLDHMVKTQASHYLKMFQSNKITNEFDPVTEISALPNVQE
KINDLLSKVLVDLGMAEAANSDDRTKRIIISYFVQHVVEA
VMVTMVQMYNDRSNLLVVGGLFYNVKLNHLLANSIKGQLC
VMPLAGDQGAALGVYQAYHGDLTWPEHLFWGDRNLDPLEF
INVPGMVVVTESQAFAEIASYISTHGWVNVVRGAMEFGPR
SLCHTATLSLPHAQFAEEINFANDRTAEMPMAPVMTLDQA
RDCFYDIDKIHKSAEYMIVARQYQAGVGSQLSGAAHWYPK
EKVFTGRPQITRDPLMVSLLEEFGPLINTSFNYHGVPIVR
SPEQIIDTHRKQYERNPKVLTIIVVKD

Conserved sequence at C-terminal end found only in hmC-CT and not in other CTs

(SEQ ID NO: 96)
NXXXXXXXXXXXXXXXTXTXXXXXXXXXXXXIXXXN.

Conserved sequence at the N-terminal end found only in hmC-CT and not in other CTs

(SEQ ID NO: 97)
XXQXA.

### Example 3. Determining the Substrate Specificity of Carbamoyltransferase

A general concern for association analysis is population stratification which can lead to spurious associations if not properly controlled. To minimize sample-specific differences between case and control cohorts, three samples from distinct sources were included and compared (FIG. 3A): two sewage microbiome samples collected at different days and one coastal microbiome sample.

To explore the substrate specificity of the CT we used single stranded DNA, double stranded DNA, single stranded RNA or nucleosides in which all the cytosine were hydroxymethylated were obtained as described below. 5-mdCTP, 5-hmdUTP and 5-hmCTP nucleosides were also used as control and obtained as described below. Reaction were performed in the presence of the substrate and freshly prepared 10 μM Iron(II) sulfate hexahydrate, freshly prepared carbamoyl phosphate, ATP and CT.

### Substrate

To obtain single stranded DNA 5-hmC: [1] single-stranded DNA oligos containing 5-hmdC were used at 1.6 μM per I reaction (sequence : 5′-TGTCCGATAGACT{5-hmdC}TACGCA) (SEQ ID NO:24). T4gt genomic DNA with 10 minutes incubation at 95° C. was performed to denature the double stranded DNA. DNA was used at 0.38 nM per reaction.

To obtain double stranded DNA 5-hmC:

[1] double stranded DNA oligos containing 5-hmdC were used at 1.6 μM per reaction (sequence : 5′-TGTCCGATAGACT{5-hmdC}TACGCA (SEQ ID NO:24) and 5′-AACTCGCCGAGGATTT{5-hmdC}TAC) (SEQ ID NO:25). [2] purified T4gt genomic DNA at 0.38 nM per reaction.

To obtain single stranded RNA 5-hmC: Forward and reverse DNA templates (Forward template:

(Forward template:
(SEQ ID NO: 27)
5'-GACCTAATACGACTCACTATAGGGAGTGAGAA
GATGGTCTAGGTGTTTATTGGTGATGAA
Reverse template:
(SEQ ID NO: 28)
5'-TTCATCACCAATAAACACCTAGACCATCTTCT
CACTCCCTATAGTGAGTCGTATTAGGTC)

were annealed at 95° C. for 4 minutes and slowly cooled for 20 minutes. RNA synthesis was performed with HiScribe T7 High Yield RNA Synthesis Kit. One μg of annealed DNA template was used per reaction with 1.5 μL T7 RNA Polymerase Mix. 5-hmCTP was used with the other three nucleotides ATP, UTP and GTP at 7.5 mM each. The reaction was incubated at 37° C. for 4 hours. Two uL Nuclease-free DNase I were added to each reaction to digest DNA templates, followed by incubation at 37° C. for 15 minutes. Synthesized RNA was purified with Norgen Biotek Oligo and Concentrator kit and stored at −80° C. 1.57 μM RNA was used per reaction.

Nucleotides tested were 5-hmdCTP, 5-mdCTP , 5-hmdUTP and 5-hmCTP. 0.5 mM of the corresponding nucleotide was used per reaction.

### Reaction Mix

Substrate (describe above) were added for each 50 μL reaction with 1× NEBuffer 2.1, freshly prepared 10 μM Iron(II) sulfate hexahydrate, freshly prepared 10 mM carbamoyl phosphate and 5 mM ATP. CT was added to the reaction at 7.2 μM.

### Assay

The reaction mixture was incubated at 30° C. for 3 hours before adding 2 μL Proteinase K to inactivate the enzyme. After 30 minute incubation at 37° C. with Proteinase K, DNA was purified with Zymo Oligo Clean & Concentrator Kit. For assays with synthesized single-stranded DNA oligos containing 5-hmdC, the heat-denaturing step was omitted. Purification was performed using Norgen Biotek Oligo

### Clean-up and Concentrator Kit

Genomic DNA and synthetic oligonucleotides were digested to nucleosides by treatment with the Nucleoside Digestion Mix at 37° C. for 3 hours. The resulting nucleoside mixtures were directly analyzed by reversed-phase LC/MS or LC-MS/MS without further purification Nucleoside and Nucleotide analyses were performed on an Agilent LC/MS System 1200 Series instrument equipped with a G1315D diode array detector and a 6120 Single Quadrupole Mass Detector operating in positive (+ESI) and negative (−ESI) electrospray ionization modes. LC was carried out on a Waters Atlantis T3 column (4.6 mm×150 mm, 3 μm) at a flow rate of 0.5 mL/min with a gradient mobile phase consisting of 10 mM aqueous ammonium acetate (pH 4.5) and methanol. MS data acquisition was recorded in total ion chromatogram (TIC) mode. LC-MS/MS was performed on an Agilent 1290 UHPLC equipped with a

G4212A diode array detector and a 6490A triple quadrupole mass detector operating in the positive electrospray ionization mode (+ESI). UHPLC was performed on a Waters XSelect HSS T3 XP column (2.1×100 mm, 2.5 um particle size) at a flow rate of 0.6 mL/min with a binary with a gradient mobile phase consisting of 10 mM aqueous ammonium formate (pH 4.4) and methanol. MS/MS fragmentation spectra were obtained by collision-induced dissociation (CID) in the positive product ion mode with the following parameters: gas temperature 230° C., gas flow 13 L/min, nebulizer 40 psi, sheath gas temperature 400° C., sheath gas flow 12 L/min, capillary voltage 3 kV, nozzle voltage 0 kV, and collision energy 5-65 V.

### Results

Nearly 70% of 5-hmdC were converted into 5-cmdC in the denatured T4gt genomic DNA. The CT shows very little activity on double stranded DNA. When using synthesized single-stranded DNA oligo containing an internal 5-hmdC site as substrate, the conversion rate was nearly 100%. LC-MS results demonstrated about 60% conversion of 5-hmdCTP. No activity was shown for 5-mdCTP or 5-hmdUTP. Activity is also seen on 5-hmCTP and on 5-hmC in single stranded RNA.

### Conclusion

CT is specific to 5-hmC or 5-hmdC in single stranded DNA and single stranded RNA as well as in 5-hmCTP and 5-hmdCTP. CT is not active on 5-hmdUTP or 5-mdCTP.

### Example 4. Determining the Context Specificity of Carbamoyltransferase on DNA Substrate for Mapping

To explore the sequence context specificity of CT on DNA substrate we used a mixture of Lambda (C) XP12 (5-mC) and T4gt (5-hmC) phage genomic DNA and treated the mixture with CT. APOBEC deaminates C, 5-mC and 5-hmC and after sequencing, the deaminated product is read as T. Deamination by APOBEC reveals whether the nucleoside has been protected by carbamoylation and to which degree it has been protected. As a control, the mixture is subject to APOBEC without prior treatment with the CT.

### Reaction Mix and Assay

1 μg genomic DNA mixture (Lambda:XP12:T4gt=1:1:1 by molarity) was sheared to 300 bp in 130 μL of TE buffer (10 mM Tris pH 7.5, 1 mM EDTA) using Covaris S2 Focused Ultrasonicator. 1.3 μL of 10 mg/mL RNase A was added and incubated at 37° C. for 30 minutes to remove RNA. To remove EDTA, the sheared DNA was purified with Zymo Oligo Clean & Concentrator Kit and eluted in 50 μL of 1 mM Tris buffer (pH 7.5).

One reaction of NEBNext Ultra II DNA Library Prep Kit for Illumina was used for 1 μg of input DNA. The DNA libraries were purified with 1X volume of NEBNext® Sample Purification Beads (New England Biolabs, Ipswich, MA) and eluted with 40 μL of 1 mM Tris buffer (pH 7.5). Libraries were subjected to CT treatment: Libraries were subjected to 10 minutes incubation at 95° C. to denature double stranded DNA. 0.38 nM denatured DNA was used for each 50 μL reaction with 1× NEBuffer 2.1, freshly prepared 10 μM Iron(Il) sulfate hexahydrate (Sigma-Aldrich, St. Louis, MO), freshly prepared 10 mM carbamoyl phosphate and 5 mM ATP. CT was added to the reaction at 7.2 μM. The reaction mixture was incubated at 30° C. for 3 hours before adding 2 μL Proteinase K to inactivate the enzyme. After 30 minutes incubation at 37° C. with Proteinase K, DNA was purified with Zymo Oligo Clean & Concentrator Kit.

Purified DNA samples were heated at 90° C. with formamide to generate single-stranded fragments before the deamination reaction. One uL APOBEC was added per reaction to both CT-treated or control (untreated) samples. The reaction mixture was incubated at 37° C. overnight. Samples were purified using Zymo Clean & Concentrator kit and pair-end sequenced (75 bp x2) with Illumina MiSeq.

### Results

Results obtained on Lambda and XP12 are similar between the CT treated and control samples indicating that CT does not protect C and 5-mC from deamination presumably because C and 5-mC are not substrate for CT. For T4gt, protection of 5-hmC can be observed for the CT treated sample compared to control. This result indicates that the CT can protect the original 5-hmC from deamination by APOBEC. 5-hmC in all sequence contexts are protected indicating that the CT has little or no context specificity.

1. A method for modifying hydroxymethylcytosines (hmC) in a nucleic acid, comprising:
(a) combining:
i. an aliquot of a sample comprising nucleic acid obtained from a eukaryotic cell;
ii. a hydroxymethylcytosine carbamoyltransferase (hmC-CT); and
iii. a carbamoyl phosphate substrate, to product a reaction mixture; and
(b) incubating the reaction mixture to modify the hmC in the nucleic acid with the carbamoyl phosphate substrate.

2. The method of claim 1, wherein the carbamoyl phosphate substrate comprises a tag or a chemically reactive group that is capable of participating in an azide-alkyne cycloaddition reaction.

3. (canceled)

4. (canceled)

5. The method of claim 1, further comprising:
sequencing the modified nucleic acid of (b) or an amplification product thereof in order to detect the modified hydroxymethylcytosine (hmC) in the nucleic acid;
determining the location of the modified hmC residues in the nucleic acid;
separating the modified nucleic acid of (b) from unmodified nucleic acid using the modified hmC residues produced in (b); and/or
visualizing the modified hmC in the modified nucleic acid of (b).

6. The method of claim 1, further comprising:
treating the nucleic acid with a deaminase, before or after step (a);
treating the nucleic acid with a methylcytosine dioxygenases before or after step (a); and/or
treating the nucleic acid with a glucosyltransferase (GT) before or after step (a).

7. (canceled)

8. (canceled)

9. (canceled)

10. The method aof claim 1, further comprising:
(c) enzymatically labelling methylcytosine (mC) in the nucleic acid with a substrate that differs from the carbamoyl substrate in (a); and
(d) determining the presence and/or location of mC and hydroxymethylcytosine (hmC) in the nucleic acid.

11. (The method of claim 1, wherein the carbamoyl phosphate substrate comprises a chemically reactive group and the method further comprises:
(c) adding a functional group to the hydroxymethylcytosine (hmC) in the nucleic acid of (b) via a reaction with the chemically reactive group, wherein the functional group is optionally selected from an optically detectable label, a bulky group that can be detected by nanopore sequencing, and an affinity tag.

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. A method comprising:
(a) combining:
i. a sample comprising hydroxymethylcytosine ribonucleotides (hmrC) or hydroxymethylcytosine deoxyribonucleotides (hmdC);
ii. a hydroxymethylcytosine carbamoyltransferase (hmC-CT); and
iii. a tagged carbamoyl phosphate, to product a reaction mixture and
(b) incubating the reaction mixture to modify the hmrC or hmdC.

27. A method comprising:
(a) combining:
i. a pool of nucleoside triphosphates comprising hydroxymethylcytosine ribonucleotides (hmrC) or hydroxymethylcytosine deoxyribonucleotides (hmdC);
ii. a hydroxymethylcytosine carbamoyltransferase (hmC-CT);
iii. a carbamoyl phosphate substrate;
iv. a nucleic acid template; and
v. a polymerase, to product a reaction mix, and
(b) incubating the reaction mix to produce a nucleic acid product that contains modified cytocines.

28. (The method of claim 27, wherein the polymerase is selected from an RNA polymerase, a DNA polymerase, and a reverse transcriptase.

29. (canceled)

30. The method of claim 27, wherein the nucleic acid product is selected from an aptamer, a DNA primer, adapter, messenger RNA, siRNA and a guide RNA.

31. (canceled)

32. (canceled)

33. (canceled)

34. The method of claim 1, wherein the hydroxymethylcytosine carbamoyltransferase (hmC-CT) has an amino acid sequence that is least 80% identical to any of SEQ ID NO: 1, 29-47, 49 or 96-97.

35. The method of claim 1, wherein the hydroxymethylcytosine carbamoyltransferase (hmC-CT) has an amino acid sequence that is least 80% identical to any of SEQ ID NO: 1, 29-47, 49 or 96-97 and has a glutamine (Q) at a position corresponding to position 169 in SEQ ID NO:1.

36. The method of claim 35 wherein the hydroxymethylcytosine carbamoyltransferase (hmC-CT) has an amino acid sequence that is least 80% identical to any of SEQ ID NO: 1, 29-47, 49 or 96-97 and further comprising has at least one of a tyrosine (Y) at a position corresponding to position 170 in SEQ ID NO: 1 or an alanine (A) corresponding to position 171 in SEQ ID NO: 1.

37. The method of claim 1, wherein the hydroxymethylcytosine carbamoyltransferase (hmC-CT) has an amino acid sequence that is least 80% identical to any of SEQ ID NO: 1, 29-47, 49 or 96-97 and does not have a serine (S), arginine (R), alanine(A), tyrosine(T) if adjacent to an serine (S), lysine (K), glycine (G), or glutamic acid (E) at a position corresponding to position 169 in SEQ ID NO:1.

38. (Amended herein) The method of claim 1, wherein the hydroxymethylcytosine carbamoyltransferase (hmC-CT) has one or more amino acids at positions in any of SEQ ID NO: 1, 29-47, 49 or 96-97 corresponding to amino acids selected from the group consisting of: asparagine (N) corresponding to position 393 in SEQ ID NO: 1, valine (V) or phenylalanine (F) corresponding to position 395 in SEQ ID NO: 1, threonine (T) corresponding to position 409 in SEQ ID NO: 1, aspartic acid (D) or proline (P) corresponding to position 416 in SEQ ID NO: 1, asparagine (N) corresponding to position 428 in SEQ ID NO: 1, and methionine (M) corresponding to position 434 in SEQ ID NO:1.

39. The method of claim 38, wherein the hydroxymethylcytosine carbamoyltransferase (hmC-CT) has two or more residues at positions in any of SEQ ID NO: 1, 29-47, 49 or 96-97 corresponding to amino acids selected from the group consisting of: asparagine (N) corresponding to position 393 in SEQ ID NO: 1, valine (V) or phenylalanine (F) corresponding to position 395 in SEQ ID NO: 1, threonine (T) corresponding to position 409 in SEQ ID NO: 1, aspartic acid (D) or proline (P) corresponding to position 416 in SEQ ID NO: 1, asparagine (N) corresponding to position 428 in SEQ ID NO: 1, and methionine (M) corresponding to position 434 in SEQ ID NO:1

40. The method of claim 39 wherein the hydroxymethylcytosine carbamoyltransferase (hmC-CT) has three or more residues at positions in any of SEQ ID NO: 1, 29-47, 49 or 96-97 corresponding to amino acids selected from the group consisting of: asparagine (N) corresponding to position 393 in SEQ ID NO: 1, valine (V) or phenylalanine (F) corresponding to position 395 in SEQ ID NO: 1, threonine (T) corresponding to position 409 in SEQ ID NO: 1, aspartic acid (D) or proline (P) corresponding to position 416 in SEQ ID NO: 1, asparagine (N) corresponding to position 428 in SEQ ID NO: 1, and methionine (M) corresponding to position 434 in SEQ ID NO:1.

41. A composition comprising: a tagged carbamoyl phosphate having the formula 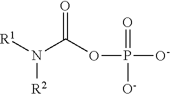
wherein:
(i) the R¹ and R² in Formula 1 independently of each other may be an H or a tag (T) comprising a chemically reactive group (C) a functional group (F) and/or a linking group (L) where the linking group may be positioned between the carbamoyl group and the chemically reactive group and /or between the chemically reactive group and the label; and
(ii) wherein the chemically reactive group (C) is selected from a succinimidyl ester, a maleimide, an amine, a thiol, an alkyne, or an azide, a carbonyl; a carboxyl; an active ester, e.g., a succinimidyl ester; a maleimide; an amine; a thiol; an alkyne, an azide; an alkyl halide; an isocyanate; an isothiocyanate; an iodoacetamide; a 2-thiopyridine; a 3-arylproprionitrile; a diazonium salt; an alkoxyamine; a hydrazine; a hydrazide; a phosphine; an alkene; a semicarbazone; an epoxy; a phosphonate; and a tetrazine.

42. The composition of claim 41, further comprising a functional group in the tag, wherein the functional group is optionally selected from an optically detectable moiety and an affinity binding moiety.

43. (canceled)

44. (canceled)

45. (canceled)

46. (canceled)

47. The composition of claim 41, wherein the tag contains a linking group (L), wherein the linking group is selected from the group consisting of: straight or branched chain alkylene group with 1 to 300 carbon atoms, a photocleavable linker, a saturated or unsaturated bicycloalkylene group, a divalent heteroaromatic group; and an oligonucleotide.

48. The composition of claim 41, wherein R1 or R2 is a group that is capable of participating in an azide-alkyne cycloaddition reaction.

49. The composition of claim 48, wherein R1 or R2 is azido or propargyl.

50. The composition of claim 41, further comprising a hydroxymethylcytosine carbamoyltransferase (hmC-CT), wherein the hmC-CT is optionally fused to an affinity binding domain or a DNA binding protein.

51. (canceled)

52. (canceled)

53. (canceled)

54. The composition of claim 50, wherein the hydroxymethylcytosine carbamoyltransferase (hmC-CT) and optionally the tagged carbamoyl phosphate is lyophilized.

55. (canceled)

56. (canceled)

57. (canceled)

58. The composition of claim 41 wherein the hydroxymethylcytosine carbamoyltransferase (hmC-CT) has at least 80% or 90% sequence identity to SEQ ID NO: 1, 29-47, 49 or 96-97.

59. A kit comprising:
i. a hydroxymethylcytosine carbamoyltransferase (hmC-CT); and
ii. a tagged carbamoyl phosphate.

60. The kit of claim 59, wherein the tagged carbamoyl phosphate comprises a chemically reactive group and optionally a functional group and a linker.

61. (canceled)

62. (canceled)

63. The kit of claim 59, wherein the tagged carbamoyl phosphate comprises a functional group, and the functional group is optionally selected from an affinity tag and a detectable moiety.

64. (canceled)

65. The kit of claim 59, further comprising in the same or separate containers, one or more reagents selected from carbamoyl phosphate, a TET family enzyme or mutant thereof, a glucosyltransferase (GT), a deaminase, and a helicase.

66. The kit of claim 59, wherein the kit further comprises a reagent comprising an optically detectable label, a bulky group that can be detected by nanopore sequencing, an affinity tag, linked to a group that is capable of reacting with the tagged carbamoyl phosphate substrate.

67. A method for distinguishing hydroxymethylcytosine (hmC) from methylcytosine (mC) in a nucleic acid molecule, comprising:
(a) placing in a reaction mixture: the nucleic acid molecule; a hydroxymethylcytosine carbamoyltransferase (hmC-CT) and carbamoyl phosphate substrate;
(b) modifying hmC in the nucleic acid molecule to form a carbamoyloxymethylcytosine (cmC) or tagged cmC;
(c) detecting the cmC or tagged cmC in the nucleic acid molecule; and
(d) distinguishing hmC from mC.

68. The method of claim 67, wherein the carbamoyl phosphate is tagged and the tag comprises a functional group selected from a detectable moiety, an affinity binding moiety, a blocking moiety, and a bulky moiety.

69. The method of claim 67, wherein the nucleic acid is chromosomal DNA and/or mRNA and the tag contains a functional group that comprises a dye for detecting the location of hydroxymethylcytosine (hmC) in vivo or in vitro.

70. (canceled)

71. (canceled)

72. A method for obtaining nucleic acid modifying enzymes, comprising:
(a) obtaining phage nucleic acid from an environmental sample from which phage particles have been enriched;
(b) identifying whether the phage nucleic acid has modified nucleotides;
(c) performing a contig analysis of the phage nucleic acid for sequences encoding enzymes capable of modifying the phage nucleic acid; and
(d) obtaining nucleic acid modifying enzymes.

73. A method for determining the presence of cytosine modifications in nucleic acid samples obtained from a biological fluid or a cell lysate, wherein the method comprises:
(a) adding a carbamoyl group to any hydroxymethylcytosines (hmC) in the nucleic acid samples, and
(b) detecting the presence of carbamoyloxymethylcytosine (cmC).in the nucleic acid.

74. The method of claim 73, wherein (a) further comprises adding a hydroxymethylcytosine carbamoyltransferase (hmC-CT) to the nucleic acid sample.

75. (canceled)

76. (canceled)

77. (canceled)

78. The method of claim 73, wherein the carbamoyl group is tagged, and (a) further comprises enriching the nucleic in the biological fluid or cell lysate by immobilizing the nucleic acids on a matrix by means of the carbamoyloxymethylcytosine (cmC) in the nucleic acid

79. (canceled)

80. The method of claim 73, further comprising amplifying and/or sequencing the nucleic acids for detecting the presence of the carbamoyloxymethylcytosine (cmC).

81. (canceled)

82. (canceled)

83. A method for determining the location of modified cytosines (C) in a nucleic acid in a sample, comprising:
(a) reacting an aliquot of the sample containing double stranded nucleic acid with (i) a glucosyltransferase (GT) for adding a sugar to 5-hydroxymethylcytosine (5-hmC), followed by (ii) a TET protein for oxidation of 5-methylcytosine (5-mC) and (iii) denaturing the nucleic acid into single strands and reacting the single stranded nucleic acid with a hydroxymethylcytosine carbamoyltransferase (hmC-CT) in the presence of a carbamoyl salt; and
(b) sequencing the glucosylated and carbamoylated single strand nucleic acid to determine which cytosines in the initial nucleic acid are unmodified or modified by a methyl or hydroxymethyl group.

84. A method for determining the location of modified cytosines in a nucleic acid in a sample, comprising:
(a) reacting an aliquot of the sample in which the nucleic acid is single stranded with a hydroxymethylcytosine carbamoyltransferase (hmC-CT) and carbamoyl phosphate;
(b) reacting the oxidized carbamoyl nucleic acid with a complementary single strand nucleic acid to form a double stranded DNA for reacting with TET protein:
(c) permitting any methylated cytosines in the nucleic acid sample to be modified by adding glucosyltransferase (GT); and
(d) performing whole genome sequencing on double stranded nucleic acid to determine the location of 5-methylcytosine (5-mC) and 5-hydroxymethylcytosine (5-hmC) in the nucleic acid.

85. (canceled)

86. (canceled)

87. (canceled)


SEQUENCE LISTING (135 sequences)

SEQ ID NO: 1 - protein, 554 aa, Artificial Sequence
  Synthetic construct
MSDLLLTLGHNASAIAISVGDDGAAKVENAYELERLTGKKSDSAFPIDAIIALKERGMDKIDRVYVSHWSPTGRVDDLKAKYWDRSIFPPHVPVITQESMNLTHHDCHAQAAMAFAGSSFPTKDTGVLVVDGFGNLAEHLSYYRVQAGGQLHLMRRWYGYGTSLGLMYQYATSFLGLKMHEDEYKLLGYGARVATIGCDMDVLNQRIFTEAQAFLKRFRSLNSFEMSPDLAGLPAVQEKWAERFAAILDDVGFKGSSSTYEARCIVGYAVQQLLEIVIRNLFMADLPKPTNLIVTGGVAFNVELNRMLLGLIPGKLCVMPLAGDQGNALGLWAFSNRRAKLDFGDLCWGRREMTLGEPGPDTPLPDGMIVVEHDTPAVYEAIAEQLKTVGFINIVRGNMEFGPRALCNTTTLARADDRAVVEEINRINGRDTVMPFAPVVSAHEWLRYFPDASRLHRSAEFMICAVQYAPGLGEQVPGAALRTVKGLYTGRPQVYSSKYEWDSVTRILDDYGLLINTSFNVHGVPICLDLKHVVHSHQFQRERNPNVRTIVIAN

SEQ ID NO: 2 - DNA, 1665 bp, Artificial Sequence
  Synthetic construct
atgagcgacctgctgctgaccctgggtcacaacgcgagcgcgatcgcgattagcgttggtgacgatggcgcggcgaaggtggagaacgcgtacgagctggaacgtctgaccggcaagaaaagcgacagcgcgttcccgatcgatgcgatcattgcgctgaaggaacgtggtatggacaaaattgatcgtgtgtacgttagccattggagcccgaccggtcgtgttgacgatctgaaggcgaaatattgggaccgtagcatctttccgccgcacgtgccggttattacccaggagagcatgaacctgacccaccacgattgccatgcgcaagcggcgatggcgtttgcgggtagcagctttccgaccaaagacaccggcgttctggtggttgatggtttcggcaacctggcggaacacctgagctactatcgtgtgcaggcgggtggccaactgcacctgatgcgtcgttggtacggttatggcaccagcctgggtctgatgtaccagtatgcgaccagctttctgggcctgaagatgcacgaggacgaatacaaactgctgggttatggcgcgcgtgttgcgaccatcggttgcgacatggatgtgctgaaccagcgtattttcaccgaggcgcaagcgttcctgaagcgttttcgtagcctgaacagctttgaaatgagcccggatctggcgggcctgccggcggttcaagagaagtgggcggaacgtttcgcggcgatcctggacgatgtgggttttaaaggtagcagcagcacctacgaggcgcgttgcattgtgggttatgcggttcagcaactgctggaaatcgttattcgtaacctgttcatggcggacctgccgaaaccgaccaacctgatcgtgaccggtggcgttgcgtttaacgtggagctgaaccgtatgctgctgggtctgattccgggcaagctgtgcgtgatgccgctggcgggtgatcagggtaacgcgctgggtctgtgggcgttcagcaaccgtcgtgcgaaactggactttggtgatctgtgctggggccgtcgtgagatgaccctgggtgaaccgggtccggacaccccgctgccggatggtatgatcgtggttgagcacgacaccccggcggtgtatgaggcgattgcggaacaactgaagaccgttggcttcatcaacattgtgcgtggtaacatggaatttggtccgcgtgcgctgtgcaacaccaccaccctggcgcgtgcggatgatcgtgcggtggttgaggaaatcaaccgtattaacggtcgtgacaccgttatgccgtttgcgccggtggttagcgcgcacgaatggctgcgttactttccggatgcgagccgtctgcaccgtagcgcggagtttatgatctgcgcggtgcagtatgcgccgggtctgggtgaacaagtgccgggtgcggcgctgcgtaccgtgaagggtctgtacaccggccgtccgcaggtttacagcagcaaatatgagtgggacagcgtgacccgtatcctggacgattacggtctgctgattaacaccagcttcaacgtgcacggcgttccgatctgcctggatctgaaacacgtggttcacagccaccagtttcaacgtgaacgtaacccgaacgttcgtaccatcgtgattgcgaactaa

SEQ ID NO: 3 - protein, 24 aa, Artificial Sequence
  Synthetic construct
LINTSFNYHGVPIVLDEQIIHHFM

SEQ ID NO: 4 - protein, 21 aa, Artificial Sequence
  Synthetic construct
DRVIIAYYVQRVLESVVLKLK

SEQ ID NO: 5 - protein, 27 aa, Artificial Sequence
  Synthetic construct
SDLYKPKNLILSGGVFYNVKLNNILDK

SEQ ID NO: 6 - protein, 13 aa, Artificial Sequence
  Synthetic construct
MPLAGDQGAALGA

SEQ ID NO: 7 - protein, 58 aa, Artificial Sequence [sequence omitted]

SEQ ID NO: 8 - protein, 26 aa, Artificial Sequence
  Synthetic construct
KRRWKKNFIAVSAANRFKKISSSGAL

SEQ ID NO: 9 - protein, 6 aa, Artificial Sequence
  Synthetic construct
HHHHHH

SEQ ID NO: 10 - protein, 8 aa, Artificial Sequence
  Synthetic construct
DYKDDDDK

SEQ ID NO: 11 - protein, 9 aa, Artificial Sequence
  Synthetic construct
YPYDVPDYA

SEQ ID NO: 12 - protein, 10 aa, Artificial Sequence
  Synthetic construct
EQKLISEEDL

SEQ ID NO: 13 - protein, 14 aa, Artificial Sequence
  Synthetic construct
GKPIPNPLLGLDST

SEQ ID NO: 14 - protein, 8 aa, Artificial Sequence
  Synthetic construct
WSHPQFEK

SEQ ID NO: 15 - protein, 38 aa, Artificial Sequence
  Synthetic construct
MDEKTTGWRGGHVVEGLAGELEQLRARLEHHPQGQREP

SEQ ID NO: 16 - protein, 15 aa, Artificial Sequence
  Synthetic construct
KETAAAKFERQHMDS

SEQ ID NO: 17 - protein, 51 aa, Artificial Sequence
  Synthetic construct
TNPGVSAWQVNTAYTAGQLVTYNGKTYKCLQPHTSLAGWEPSNVPALWQLQ

SEQ ID NO: 18 - DNA, 20 bp, Artificial Sequence
  Synthetic construct
ttgctgagtttcacgcttgc

SEQ ID NO: 19 - DNA, 20 bp, Artificial Sequence
  Synthetic construct
aaaaccgcttgtggattgcc

SEQ ID NO: 20 - DNA, 20 bp, Artificial Sequence
  Synthetic construct
tcgcgaaacggttttccaag

SEQ ID NO: 21 - DNA, 20 bp, Artificial Sequence
  Synthetic construct
aaagcgcttgacccaacaac

SEQ ID NO: 22 - DNA, 20 bp, Artificial Sequence
  Synthetic construct
tgcgatgttggattcgttgg

SEQ ID NO: 23 - DNA, 20 bp, Artificial Sequence
  Synthetic construct
acaacccgccataatggaac

SEQ ID NO: 24 - DNA, 20 bp, Artificial Sequence
  Synthetic construct
  misc_feature (14)..(14): C is 5-hmdC
tgtccgatagactctacgca

SEQ ID NO: 25 - DNA, 20 bp, Artificial Sequence
  Synthetic construct
  misc_feature (17)..(17): C is 5-hmdC
aactcgccgaggatttctac

SEQ ID NO: 26 - DNA, 45 bp, Artificial Sequence
  Synthetic construct
  misc_feature (20)..(20): C is 5-hmdC
acacccatcacatttacaccgggaaagagttgaatgtagagttgg

SEQ ID NO: 27 - DNA, 60 bp, Artificial Sequence
  Synthetic construct
gacctaatacgactcactatagggagtgagaagatggtctaggtgtttattggtgatgaa

SEQ ID NO: 28 - DNA, 60 bp, Artificial Sequence
  Synthetic construct
ttcatcaccaataaacacctagaccatcttctcactccctatagtgagtcgtattaggtc

SEQ ID NO: 29 - protein, 541 aa, Artificial Sequence
  Synthetic construct
MKTALALTLGHNSSAVLIQDGEIIAGYEQERFSAKKSDSAFPKDAIKELALRYDLPSNTTVCVGHWFLDHQLPPVANKYWDPDHIREMFPDGEVLSLDAQFSHHDSHLEAAMVFAGKNFAPSYHALVLDGFGSSGECISVYAVNKGSYRLLERWFGFDKSLGMLYQYATAFLGMKMHNHEYKMLAYEVHLHALNYDVKRLDELVAKCADEWLTKMFRHQIHPGTDPLISLEALPNIQNTIEQMLGDVLIELDAGDVDIHDKRCIISYFVQHVVESVVLSLAKVYSPENLLLVGGLFYNVKLNNLLASKVSGRTCIMPIAGDQGAGIGVYQRYFGDLKWPEHLFWGYRNLDFQSDDESMVIVDTMEDALPLLAAELESVGFVNLVRGAMEYGPRALCNTTTLAIPDRDVGATINRINDRTNEMPFALFMSKSQADDLFVDCDKVYKSLEYMICTRNFKPGKAESCLGGAHYYPDLDVYTCRPQITDDEHLLVLLDKFGPLINTSYNYHGVPIVLGEEQIKHTHKMQRQTAPDVAFKTIIVRK

SEQ ID NO: 30 - protein, 550 aa, Artificial Sequence
  Synthetic construct
VQPKLNGLNNGISGMKALLLTLGHNSSAILIEDNKVVWGYETERISGLKSDSRFPMPFIEKYLGKRPEFDVVYVTHWATDGVLSSMSAKHWDPSYFDGKPIRTLSIDRSHHDTHMSAAMCYAGKEFFKHKHSVHGLIIDGFGTLGEHLSIYDLSDGTPKLIKRIHGYETSLGLWYQYATAFMGMKMHEDEYKLLGYETHCHGPIGDQLIHEADLYALEWIDKLGKSIYKSIYDPMYSLDALGAVKTNVFKHLAKVCRQYNLADPTESVARSILALYVQRVLESVVLNLIGHYNMKNVILSGGCFYNVKLNKRIIDEIDGQVCIYPLAGDQGNALGLYAMDHPEFIFPDNLNWGIRKLKNVGEIRGIFYADSYDEATEMITSSLAHHGYVNLVRGAMEFGPRAMCNTSTLAIPTMDVVQSINTMNNRNTVMPMAPVMTEYMYKTMFERTSQVWRSQHHMITALEYVEYPPSHLLGIAHEYLMPHHHHTGRPQVTHDPFMVKILDHIGHPLINTSFNYHGMPIALGMEEVISNHRMQQQREPSISTVVITNA

SEQ ID NO: 31 - protein, 502 aa, Artificial Sequence
  Synthetic construct
IKSNSSFPIKAICRLNDLYHLPSDVKVYVSHWFLDGTLEHANAKHWSIRLLKTLFPYASIRGINPDKCTHHAAHALSAEVFAGPDFPKDHHVLVVDGFGTQGECYSAYAYDSDISGLRVGLAYHGFDRSIGLFYQYATAYCGMKMHQHEYKMLAYETHLTEIMPCPYKVNELNEYIDLYSDNYDFVVSTYEVSLDRLSSVQAIINCVLDAYLKYVKRNRLTLTDPYGDRSKKILVSYFTQRHTENTVRKFVKSIKPTNLIVAGGVFFNVKLNKMICDMIPGKFCAMPLAGDQGAGLGVYQYYNGDLIWPDHLFWGHRDLVLDLPKGLMRKGTVEDVKNCLFSNRLVNVVRGSMEFGPRALCHTSTLGFPAKDVAERINRMNDRTNEMPFALVVTRDQADELFEDIDKVHKSLEYMIVTRNFKKGREKKYLGGAHYYPLTKEYTCRPQITDDPFMIEILNEFGPLINTSFNYHGCPIVFDQNHISHSHFMESKHELISTIIIS

SEQ ID NO: 32 - protein, 539 aa, Artificial Sequence
  Synthetic construct
MSSSIFINSAFSIISCFISSMTILSILINSAIIIHSILLYIISCFLVFNTFFDKLNTMNTLGISCGSHDAAIAAIKNNEIVFASQSERYSKNKNDLYLNRDVILDALYSLDYNVDSLCFYENILNKKTRQFFAGQYYNVFVDKPLSSWYNNIKNIYTNYKLLSKLKIHQIDHHKSHAALAFASPYNKAIVLVIDAIGEWDTISIWLYNNSLNRKLIKLESYSYPQSLGLFYTAFTDYVGLKANEEEYIFMGMAAFGKPIYKDMIRKYLFNSKGNIKINLHTGIPKDLLNININDFKTKENIASSVQDLTEEYIFKLLNKIKYYQDIYKTKNLIYSGGIALNCVANSKIQKEFSNMWIFPNPGDSGSSLGCALAYLNNKTKFDSVYLGYNICYNSICPNDISEYLLNNKICGIALGKAEFGPRALGNRSLLADPRGNEIKDKVNEIKKRDKFRPFAPIIMEEYFKEYFEVPNNIMNSSYMQYVYKCKYPDKFPAIVHIDGTSRVQTINEKQNAFVYNVLKSFYNKTGCPMLLNTSLNIKG

SEQ ID NO: 33 - protein, 552 aa, Artificial Sequence
  Synthetic construct
MAESVERQEQEMKALLLTLGHNSSAVMIENDQIVWGYETERLSGLKSDSRFPMPWIEKLPNTDLRNIDIVYVTHWAPDGQLSSMSRKHWDPFYFDGVPIRTLSADRTHHDTHMAAADCYAGPAFTKHRKPGTFGFVVDGFGTMGEHFSVYEYQSDHTPKLVRRVHGYGTSLGLWYQYATSFMGLKMHEDEYKLLGYEVHCPLNLINDIDMAAAVKADEWIDEMKKSIYGSKYDPLYDLRALENVKNKIFEHLNQKCKDFGVTDPTSHEGRCVLAYYVQAVLEGVVTTLIHQFKPKHVVLSGGCFYNVKLNKSIVDRIEGQTCVYPLAGDQGNALGLYFMDNPQFCMPNHFNWGKRDLRDVGEVKGLVCVKDTNSALKQLTDRLYQVGHVNLVRGSMEFGPRAMCNTSTLALPTAASVELINAMNDRNTVMPMAPVMTMAMYRSLFERWDRVWGSYRHMIVAMEYVEHPGDELRGVAHEYQVPYHHHTGRPQVVPDTDWLMGALLARFDGPLINTSFNFHGQPIAFNMESVIKNHMLQYQRDPSTFTVVITNE

SEQ ID NO: 34 - protein, 433 aa, Artificial Sequence
  Synthetic construct
MKSLLLTLGHNSSAIMVEDGKIKWGYETERISRQKSDSHFPEAVLSTMDVKQVDEVYVTHWSPDGRLSSMSAKHWHPARFENTPIRTLSVGLSHHDTHMHGAMCYAGNSFPYSAGTYGLVVDGFGTLGENLSVYNLTSGKPILKKRVHGYGTSMGLWYQYATAFMSMKMHEDEYKILGYEAHVPEDLVAKLNAAADVRASYILDEMHKSVYGSAYDPMYSLEALANIKNEIFTHLYNVCTQFSVTDPSSQSGRIIIGYYVQAVLESVILTVVRSLNARNLILSGGVFYNVKLNKRIIDECEGLVCVYPLAGDQGNAIGLYYLDHPEFEFPSNLNWGHRTLRDPGNIPNMIFARSEEEAGMLIHRELAQGIGYINLVRGNMEFGPRAMCNTSTIAMPTRRCVQAINAANNRNTFMPMAPVMTRECYRQLFENTD

SEQ ID NO: 35 - protein, 445 aa, Artificial Sequence
  Synthetic construct
LDAQFSHHDSHLEAAMVFAGKNFAPSYHALVLDGFGSSGECISVYAVNKGSYRLLERWFGFDKSLGMLYQYATAFLGMKMHNHEYKMLAYEVHLHALNYDVKRLDELVAKCADEWLTKMFRHQIHPGTDPLISLEALPNIQNTIEQMLGDVLIELDAGDVDIHDKRCIISYFVQHVVESVVLSLAKVYSPENLLLVGGLFYNVKLNNLLASKVSGRTCIMPIAGDQGAGIGVYQRYFGDLKWPEHLFWGYRNLDFQSDDESMVIVDTMEDALPLLAAELESVGFVNLVRGAMEYGPRALCNTTTLAIPDRDVGATINRINDRTNEMPFALFMSKSQADDLFVDCDKVYKSLEYMICTRNFKPGKAESCLGGAHYYPYLDVYTCRPQITDDEHLLVLLDKFGPLINTSYNYHGVPIVLGEEQIKHTHKMQRQTAPDVAFKTIIVRK

SEQ ID NO: 36 - protein, 544 aa, Artificial Sequence
  Synthetic construct
TLGHNSSAIFVEDGQVICGYEEERLSEIKSDSAFPKKAIDLITKHYCNRFDNVFVGHWFNNGNLTDCKYWDEKYILQFVKDPRCIKSLNVDFTHHDSHRVAAECFYKNYSQQLLDENDYCIVADGFGTFGESLSIYGYHNGKAHVKSRVFDYINSLGLLYQYATLYLGMKMHNHEYKMLGYEAHIHEHFTQDEIFIMDGWADLQTDKILKSMNSVSLKQTDPIVNTDALDFVRSSIFDLLDTFCDKFDIKSVENDVIEKKRVGVSYYVQKIVETVILDIVASLGKVKTLLLSGGLFYNVKLNNKISKLVGNLCILPVAGDQGAGLGVYHHYVGDLQFPRSLCIGHRFLDADDFKDIDGIETFENADEMYHRIEECLADNGFVNVVKSSMEFGPRALGSTSTIAYPEVDVVKIINELNDRTFVMPMAPMMTQEQFNMLATSGFNVIGSNEFMVSTVDVHKYFIEGIEGAVHRYVDAATCRPQIVDGNPFYKTLCSNLGPLINTSFNYHGVPIVFDSKQIIHSHQCQQKNANGKYNVITLIYTGSN

SEQ ID NO: 37 - protein, 535 aa, Artificial Sequence
  Synthetic construct
MKSLVLTLGHNSSAIVIEDGHILGGYEEERLSGIKSDSSFPIKAICRLNDLYHLPSDVKVYVSHWFLDGTLEHANAKHWSIRLLKTLFPYASIRGINPDKCTHHAAHALSAEVFAGPDFPKDHHVLVVDGFGTQGECYSAYAYDSDISGLRVGLAYHGFDRSIGLFYQYATAYCGMKMHQHEYKMLAYETHLTEIMPCPYKVNELNEYIDLYSDNYDFVVSTYEVSLDRLSSVQAIINCVLDAYLKYVKRNRLTLTDPYGDRSKKILVSYFTQRHTENTVRKFVKSIKPTNLIVAGGVFFNVKLNKMICDMIPGKFCAMPLAGDQGAGLGVYQYYNGDLIWPDHLFWGHRDLVLDLPKGLMRKGTVEDVKNCLFSNRLVNVVRGSMEFGPRALCHTSTLGFPAKDVAERINRMNDRTNEMPFALVVTRDQADELFEDIDKVHKSLEYMIVTRNFKKGREKKYLGGAHYYPLTKEYTCRPQITDDPFMIEILNEFGPLINTSFNYHGCPIVFDQNHISHSHFMESKHELISTIIIS

SEQ ID NO: 38 - protein, 314 aa, Artificial Sequence
  Synthetic construct
LKFMKENNMKLLLTLGHNSSAILVDDQKVLCGYEEERLSGVKSDSSFPSLSIKKILEFYPQAKMEVDEVCVSHWFWSWDLVENKYYQPKFIAANFPKASVKSVSYYETHHDLHAKSLWNFLDGDNSGLTVVADGFGNFGECLSIYIDGKLYHRSYEVSRSLGIMYQYAIRYLGMKENQDEYKLLGYEQNVSEAFKNNITEDIIEVSNVMYKSLTNKVAMKKDMLAELDKAWNYWRVTFSEIDDSEQDRAKIAYFVQQILENVMIKIIRSFNIKNIKVTGGVFYNVKLNNLILRYADKFEAYPLAGDQGCALGFT

SEQ ID NO: 39 - protein, 422 aa, Artificial Sequence
  Synthetic construct
LKFMKENNMKLLLTLGHNSSAILVDDQKVLCGYEEERLSGVKSDSSFPSLSIKKILEFYPQAKMEVDEVCVSHWFWSWDLVENKYYQPKFIAANFPKASVKSVSYYETHHDLHAKSLWNFLDGDNSGLTVVADGFGNFGECLSIYIDGKLYHRSYEVSRSLGIMYQYAIRYLGMKENQDEYKLLGYEQNVSEAFKNNITEDIIEVSNVMYKSLTNKVAMKKDMLAELDKAWNYWRVTFSEIDDSEQDRAKIAYFVQQILENVMIKIIRSFNIKNIKVTGGVFYNVKLNNLILRYADKFEAYPLAGDQGCALGFTNVKYDNLFWGKRSVGNIDVNEVIDNIKTYGYSELFHGDMEFGPRALCNTTCLAVPTSEMVEKINAQNGRDTVMPMAPVVTEKFMNKWFNDIEKVGKSKNFMIIAYDYK

SEQ ID NO: 40 - protein, 305 aa, Artificial Sequence
  Synthetic construct
QKFIKKEIKMKLLLTLGHNSSAILVEGEKVVCGYEEERLSGVKSDSSFPSLSIKKILEFYPQAKLLVEEVCISHWFWSWDLVENKYYQPKFIKANFPKAKVTSVDFNNTHHDLHARSLWNFLDGDQSGLTIVADGFGNFGECLSVYVDGILKHRSYEVAKSLGIMYQYAIRYLGMKENQDEYKLLGYEQNVSEAFKKQIAGSITEVSSHLYKELSCVQNGMKKDMLAELDKAWNYWKVTFSEVDDADQDRAKIAYFTQQVLEQVMMAIIQDYDIKNIKVSGGVLLQKIHLQKYICLLFLLVLFLL

SEQ ID NO: 41 - protein, 540 aa, Artificial Sequence
  Synthetic construct
GGFTMNLCLSLGHNSSAVLVDDYGRIIIGYENERLSGIKSDSHFPQDAIKEIGKYYYLSLIDRVYVSHWATFGSVEEMSAKHWRPDILSELCPNAILQRDVDHHECHVSALRAFTSETFNWEIVADGFGNFNETMSIYHKGALIHRCFGYEKSLGLFYQYATAYMGLKMNQDEFKLLGYESKIKEVVSNKCIVEILSVAQKTADKFFRSIIDTSLEPKYDAVAGLEALPNLRVKIENNLDNLLYVKLKDVYSASRGSDSDRVVIAFYVQSVIESVLRKVVLHFGMDEVALTGGIFMNVKLNNIISKLVNKISVMPICGDQSGGLGAYEYYNGNLQWPDHLFWSDRGGLFTLDDGDPDMVVFDEEVDALRYISLCLNADRIVNLVQGKGEFGARALGNTSTLALPTSNNVEYINHLNQRSTIMPMAGMISPKALSNYTDADKVHKSLEYMIITLDAKKVDSSTEGCHHNHPINGSITNRVQLVDNGSLVGEIVKRFDCLINTSFNVHGVPIVLTSAQVQKSHDSQKSLDYFDRMVTVIVRN

SEQ ID NO: 42 - protein, 338 aa, Artificial Sequence
  Synthetic construct
LGIMYQYAIRYLGMKENQDEYKLLGYEQNVSEEFKEDIQDMIEENVALSYSFLKTKMPMKQDMLAELDKAWNYWRVTFSEVDNAEQDRPKIAYFTQQVLEKVMMKFIESYSISNIKVTGGVFYNVKLNNKILKYASKFEAHPLAGDQGCALGFIPVRYDNLFWGIRFVSEGIDVARVIEDIEHQGYSELFHGDMEFGPRALCNTTCLALPTSEMVEKINAQNGRDTVMPMAPVVTEKFAKTWFKDIDKVGKSKNFMIIAYDYKGMNENIKGAAHIDSDRDVYTGRFQLATDPRIIKILNHFGGILINTSLNAHGQPIIYDQNDFAMMRYIQDEVQKNR

SEQ ID NO: 43 - protein, 105 aa, Artificial Sequence
  Synthetic construct
FRPFAPAILEEFADSYFDGPMNEYMQYTSQAKHDYKSVTHVDGSARVQVVKKNCRSILRPILEEFYNQTKCPMLLNTSLNIKGQPMVNTIEHAKEFQNKYKVKVF

SEQ ID NO: 44 - protein, 283 aa, Artificial Sequence
  Synthetic construct
KMRYIGLHAYGHDANKTTTILENGVVVENKHIEYERLLREKRVKFDLIDKSTFLESEENSQINISISGHHNLQKIMGDEDFKIDYLPSINRKLDDEKYITHPILGNKTMLISHHLAHASYAYYTRPYYMKDSDVFTYDGRGIFTDSTFHNNTKSITNDVIGIGMLWDLIGRCINRDAFTEGKVMGLSAYGKYNSKLESIFLQYIKEYTTKDNTDIAWFPTEEEHIKLLKEYSELLKPYDNKNIAATLQRFSEVYVLEYLKRYKTSDNLCIGGGVGLNGYINQK

SEQ ID NO: 45 - protein, 148 aa, Artificial Sequence
  Synthetic construct
IFGASVIFSNHSPTMKDYLNERVKHREWYRPFAPAILKEHVTEWFENITDAPNMLKIARYKKGMGEKVPAVCHIDYTGRLQTVTKESNEHFYNLISAFNGISGVPILLNTSFNDNGEPIVDSPQDALKTFNNTNIDILVINNYVYEKE

SEQ ID NO: 46 - protein, 575 aa, Artificial Sequence
  Synthetic construct
MKKALMITLGHNSSALFFDGENTVGYEQERLDKIKSSSAFPKDAIEKIKEQYDIKDSYLFVSHWYDSFDLKTVDYSKHVDNIFLEKLVNDFNLTYIPLSEDFTHHDAHAYSSLSFLLNFMNKEKTKLLVGDKIHFLVVDGFGNRQEVVSLYSLNKKDLFVKNNNLKKIASFGGYFKSLGLMYQNATSYCGMKENQDEYKFLGYESHITSVLNSTDLLNLDTLLDDRIRDIFDNMFVEYRYCVVESFINVGDLRLVKKYWYEIFDSILSNIGIKNDKNFVSRVIIGYSIQRIIEDILLKIVKMYDIKNLCVSGGVFYNVKLNNRLLNSVPGIFSAMPLAGDQGAAIGMYKKYVGDFYFKNMCFGIRDEFDYSLLKQVDFKDKIFVTKNRHDVVEKIAELLKQDNIVNFVEGNMEFGPRALCHTSTLSLPSSTNVDYINTLNKRNTVMPMAPVMLQDFVHTFFDTKTYDRIIGSDMFMIVTLNYKEFDEKYNGVSHKYPKLDVYSGRPQVVSSGDNKIVKNVLENLSDYTKCLVNTSFNVHGNPILYDYKSIFEDFVFQCNQAVENNIKIPYLVLYV

SEQ ID NO: 47 - protein, 541 aa, Artificial Sequence
  Synthetic construct
MEIIIMPKYSLAISLGHNSSAVLIEDGHILIGFENERISKVKSDSAFPKLAILEIGKFYDLHSVEDIYVSHWEVTGNVDNMSLKHWRKDFLLTACPQARIHADYDHHDCHVAALRTFSGGNKWEIVADGFGNFNEVISIYKDGNLLHRVFGYEKSLGLLYQYATAYLGLTMNQDEYKLLGYESKIGLTIDANRLSILQDEIFNYADKYFKAIIKPKIVPKYDSVAGLDALVNIRLNITNHLDYILYKKLGLVKSETSISMQRIIVAFYIQSVVEIVMEKLVRAFDMKDVGLCGGLFMNVKLNNRISQCVKNMDIMPLCGDCGAGIGAYQIHRGDLKWPDHLYWGHRNYFLKPVGKDLICYLHFNEALQELITNLRKDNIVNFVCGSMEFGARALGHTTTFALPTVVNVHYINLLNERNTIMPMAGMMSDHCMRANYERYSIAPKSVEYMITTLDLKLGSTLEAQGVHHLDVVRGVFTNRVQVVGEGHDFYPLVEEFGTVINTSFNIHGTPIVYDADDVMKAHSFQKARDRDNRVVTIVVVA

SEQ ID NO: 48 - protein, 576 aa, Artificial Sequence
  Synthetic construct
HHHHHHMRVLGLNGWPRDFHDASAALLVDGRIAAFAEEERLTRKKHGYNTAPVQAAAFCLAQAGLTVDDLDAVAFGWDLPAMYRERLGGWPHSDSEALDILLPRDVFPRRTDPPLHFVQHHLAHAASAYYFSGEDRGAVLIVDGQGEEECVTLAHAEGGKITVLDTVPGAWSLGFFYEHVSEYTGLGGDNPGKLMGLAAHGTTVDETLSAFAFDSDGYRLNLIDPQARDPEDWDEYSVTERAWFAHLERIYRLPPNEFVRRYDPAKGRVVRDTRRDPYEYRDLAATAQAALERAVFGLADSVLARTGERTLFVAGGVGLNATMNGKLLTRSTVDKMFVPPVASDIGVSLGAAAAVAVELGDRIAPMGDTAAWGPEFSPDQVRAALDRTGLAYREPANLEREVAALIASGKVVGWAQGRGEVGPRALGQRSLLGSAHSPTMRDHINLRVKDREWWRPFAPSMLRSVSDQVLEVDADFPYMIMTTKVRAAYAERLPSVVHEDWSTRPQTVTEASNPRYHRMLTELGDLVGDPVCLNTSFNDRGEPIVSSPADALLTFSRLPIDALAVGPYLVTKDLRH

SEQ ID NO: 49 - protein, 547 aa, Artificial Sequence
  Synthetic construct
MTYALAISLGHNSSAILIQDGVVLAGYEEERFSGVKSDSKFPYQSILELKRRFDLPSDTDSFVGHWFLDAQLPAPNKYWDPDFLRSHFPNGSIDSLNADFTHHDSHLASAMVFAGEEWSDKSYTAVVADGFGSYGECLTIYAVTGQSYSVKHRVFGFEKSLGMLYQYATAFMGMKMHNHEYKMLAYEVHIGEVLDVDQIDRLDHMVKTQASHYLKMFQSNKITNEFDPVTEISALPNVQEKINDLLSKVLVDLGMAEAANSDDRTKRIIISYFVQHVVEAVMVTMVQMYNDRSNLLVVGGLFYNVKLNHLLANSIKGQLCVMPLAGDQGAALGVYQAYHGDLTWPEHLFWGDRNLDPLEFINVPGMVVVTESQAFAEIASYISTHGWVNVVRGAMEFGPRSLCHTATLSLPHAQFAEEINFANDRTAEMPMAPVMTLDQARDCFYDIDKIHKSAEYMIVARQYQAGVGSQLSGAAHWYPKEKVFTGRPQITRDPLMVSLLEEFGPLINTSFNYHGVPIVRSPEQIIDTHRKQYERNPKVLTIIVVKD

SEQ ID NO: 50 - protein, 169 aa, Artificial Sequence
  Synthetic construct
AQAISEGKVIGWFQGRLEWGPRALGNRSILGDPRRANMKDILNEKIKRRESFRPFAPSILREEVIGWFEEEADVPFMMQVFQVRQEKRGIIPATTHADGSGRLQTVHKDTNPLYHQLITAFKELTGVPIVLNTSFNENEPVVCHPREALECFLRTKMDVLVLGSWMVRR

SEQ ID NO: 51 - protein, 189 aa, Artificial Sequence
  Synthetic construct
ARLLDAGKVIGHVQGRMEFGPRALGNRSILGDARNTTMQATMNLKVKFRESFRPFAPSVLEEDIGEYFELDAPSPYMLLVAPVRADRRKAMTDAQQALFGIDKLNVPRSDIPAVTHVDYSARVQSVKASRSPRYHKIISAFKARTGYGVIVNTSFNIRGEPIVSTPQDAYRCFMFTDMDALVLEDCICL

SEQ ID NO: 52 - protein, 165 aa, Artificial Sequence
  Synthetic construct
LLADGKVVGWFQGRSEIGPRALGHRSILADPRNFNMRDYLNNEVKHREWYRPFAPVVLEEKASDFFDLSVPSPFMLLVAKVKNPEIIPSACHIDGTARVQTIRRGQDAMYYDLVKSFGERTGIPVLLNTSFNDAGEPIVETPEHALRSFISMQLDALVINDLLLL

SEQ ID NO: 53 - protein, 193 aa, Artificial Sequence
  Synthetic construct
GEVANEKVIGWFAGRMEFGPRALGARSFIGDARSPAMQAQMNLRIKFRESFRPFAPVVLREDVEHWFDHKGESPYMLVVADVKPEHLRQLSAEEQQNMKDPDLRKRVNVPRSTLPAITHVDNSARLQSIDERRHGRFCRLMREFKRQTGCGVMINTSFNIRGEPIVCTPTDAFKCLQATEMDVLVVENFILRK

SEQ ID NO: 54 - protein, 168 aa, Artificial Sequence
  Synthetic construct
ATAITEGKVIGWFQGRMEWGPRALGNRSILGDPRRADMKDILNLKIKRRESFRPFAPSILRECVTEWFEQDDDVPFMMEVFQVRPEKRAQVPATTHVDGSGRLQTVHKETNPRYHRLISDYREITGVPIVLNTSFNENEPVVCKPQEALDCFLRTKMDVLVLNDWIVT

SEQ ID NO: 55 - protein, 168 aa, Artificial Sequence; identical to SEQ ID NO: 54
  Synthetic construct

SEQ ID NO: 56 - protein, 171 aa, Artificial Sequence
  Synthetic construct
ARYIADNLVVAWFQGAMEFGPRALGNRSILANPCNAESSELVNKKIKFRESFRPFCPSILENDLQLYFSGKSKHSPYMTITYDANTHAQETIPAVIHIDNTARIQTVNESDNPIFYKLLCEFKKISGHGVVLNTSFNRSYEPMVCSPRDALATFYSSGLDVLIMGNYVIKK

SEQ ID NO: 57 - protein, 166 aa, Artificial Sequence
  Synthetic construct
ARLLARGAVIGVAAGRAELGPRALGNRSILAAPGSPDVRDRINARIKSREWYRPIAPAVRLADVDRFFAPPAVSPYMSFAADVRSDARGLVPAAVHVDGTARVQTVTDDAPLGPILSALRTLGATPCVINTSFNLRGQPLLQRASAALSALDQADLDFAWIDGWLL

SEQ ID NO: 58 - protein, 171 aa, Artificial Sequence
  Synthetic construct
AALLDGGAILAWCHGRFEWGPRALGMRSILAGPRDPAIRDRLNRAVKRREPFRPFAPAVLADDAARWFGDPAADLTPFMTTVCEVLPAARADLGAVTHVDGTARVQTVTAASSPGLHALLTALGRGGAPPIVLNTSQNGGGEPIVASEVDALGFLLAHPIDGLVVDDVLIT

SEQ ID NO: 59 - protein, 167 aa, Artificial Sequence
  Synthetic construct
CIINGGVVGWFQGRAEFGPRALGHRSIIADPRRADAKEILNLKIKRRESFRPFAPSILKEFVPDFFEDADNVPFMEKVYLIKESMRNKIPAVTHVDGTGRLQTVDKQVEPRYHGLISAFYEKTQVPILLNTSFNENEPIVNSPANALDCFLRTKMDMLVLENYIVSR

SEQ ID NO: 60 - protein, 190 aa, Artificial Sequence
  Synthetic construct
ANLINSEKVIGWFDGKMEFGPRALGSRTIIGDARSPKMQATMNIKIKFREGFRPFAPSVLSEKVSDFFEIEKESPYMLLTADVKKERRIPMTDDEHKKWGIDKLNVVRSDIPAITHVDYSARLQTVHKETNPRYHKLISKFEEKTGCAVIINTSFNVRGEPIVCSPKDAYKCFMRTDMDYLVLGNYILSK

SEQ ID NO: 61 - protein, 187 aa, Artificial Sequence
  Synthetic construct
AAALAAGEVVGWFQGRMEFGPRALGNRSILGDPRNPGMQRTMNLKIKFRESFRPFAPSVLREHLSDFFELDTDSPYMLLVAPVKEALRIPAEPGLFGIDKLMQARSSLPAVTHVDYSARIQTVDRDVHPRYHALIDAFRAQTGCAALINTSFNVRGEPIVATPEDAYRCFMRTGIDRLVVGNCVLRK

SEQ ID NO: 62 - protein, 170 aa, Artificial Sequence
  Synthetic construct
ARVLADGNPVAWFQGRMEFGPRALGGRSILGCPSVPGVADRINAQIKFRERWRPFCPSMLDSVGPQMLGTEHPAPFMTFTFEVAPGWKERVPEVVHEDGTARAQVLKREFNPRYYDLMTELERLTGNGVVLNTSLNRRGEPMICSPTDALNMFFGSDLQYLIMEDILVVK

SEQ ID NO: 63 - protein, 190 aa, Artificial Sequence
  Synthetic construct
ARHLANDKTIAWFQGRMEFGPRALGSRSIFANPATPQIQQTLNQKIKKRESFRPFAPAMLLEDYLEFYEGGNENSFMSMTDFLKKEKRLGDDFTSEETGITEQLQKSKSEIQGVTHVDYSSRIQVVSETEDPLFFKLLTEYKKITGRGILINTSFNLRGQPIVCSPDDACKCFLETDIDLLVINNFIASK

SEQ ID NO: 64 - protein, 58 aa, Artificial Sequence
  Synthetic construct
RYWELINEFKNQTGFGLLVNSSFNVRGEPIVCTPDDAFRCFMRTEMDVLVVGNFVFDK

SEQ ID NO: 65 - protein, 190 aa, Artificial Sequence
  Synthetic construct
ASLLSAGKIVGWFQGRMEFGPRALGARSILGDPRSPSMQKVMNLKIKFRESFRPFAPSVLAGDVSEWFDLEGESPYMMLVANVAEKHRRKMSAEEESLWGIEKLNAVRSDIPAVTHVDYSARIQTVHEETNPLYHALLSRFKAKTGCPVLVNTSFNVRGEPMVGSPADAFRCFMGTDIDTLVAGRCILYK

SEQ ID NO: 66 - protein, 171 aa, Artificial Sequence
  Synthetic construct
AKILAEGHVVAVVRDREEFGARALGNRSIIASPNNPEVVKTINEAIKNRDFWMPFALTILAEHTQDYLVNPKNLDSPCMTIGFDTLENKYPNIKAGTHPYDRTCRPQILEKQANPQYHDLINEFYKITDIPAVLNTSLNLHGDPICSTIEDIHRTFTHSGLNYLYINDTYL

SEQ ID NO: 67 - protein, 196 aa, Artificial Sequence
  Synthetic construct
NLLESEKVIGWFQGRMEYGPRALGARSIIGDARSTKMQSIMNLKIKYRESFRPFAPIVLKEHASSWFKIDTKHESPYMLIVADVAEEHKTRLTPEQEAKMNTSTDLCERVNIPRSTVPAVTHVDNSARLQTVDSDRNPRLHALMSTFHARTGCPVLVNTSFNVRGEPIVCTPEDALRCFMATEMDVLVLENCVILK

SEQ ID NO: 68 - protein, 196 aa, Artificial Sequence
  Synthetic construct
NLLISEKVIGWFQGRMEYGPRALGARSIIGDARSTNMQSIMNLKIKYRESFRPFAPIVLREHASAWFEIQKQHESPYMLIVADVSEQHKTRLTAEEETQMHTSSDLCERVNIPRSTIPAVTHVDHSARLQTVDKQRNARLHALMSNFFEKTSCPVLVNTSFNVRGEPIVCTPEDAFRCFMATEMDALVMENCVILK

SEQ ID NO: 69 - protein, 170 aa, Artificial Sequence
  Synthetic construct
ELSNNQVVGLFRGKMEFAPRALCNRSIIYHTKDKTMNDWLNKRFDRTEFMPFAPVTPENLAEKCFINWSESDISAKFMTVTYDCTELMKSKCPAVVHIDGTARPQIIIQNKDNFTYDLINKWYELTGEPSLVNTSFNKHEEPIVCSPADALDALRSGIIDVVCFNDRIIC

SEQ ID NO: 70 - protein, 167 aa, Artificial Sequence; identical to SEQ ID NO: 59
  Synthetic construct

SEQ ID NO: 71 - protein, 190 aa, Artificial Sequence
  Synthetic construct
ADALANDEAVGWFQGRMEFGPRALGARSILGNPRSPSMQKVLNLKVKFRESFRPFAPSVLAEHVSDWFEMDDASPYMLLVAPVAAKRRITMRSEQDALFGLDKLNVSRSDIPAVTHVDYSARVQTVHADTNPRYHRLLSMCYEKTGCPVLVNTSFNVRGEPIVCTPEDAFRCFMGTGIERLAIGNCLLTK

SEQ ID NO: 72 - protein, 170 aa, Artificial Sequence; identical to SEQ ID NO: 62
  Synthetic construct

SEQ ID NO: 73 - protein, 189 aa, Artificial Sequence
  Synthetic construct
AKVMAEENVVGLVQGRMEFGPRALGGRSIVGDARSPKMQSVMNLKIKYRESFRPFAPSCLREKVSDYFELEKESPYMLLVAQVQPSRCKGLEHEGPVALKARINQIRSDIPAITHVDYSARVQTVEREANPRYYDLIKAFGDLTGYYVIINTSFNVRGEPIVCTPEDAYRCFMRTEMDYLVLGDFILAK

SEQ ID NO: 74 - protein, 190 aa, Artificial Sequence; identical to SEQ ID NO: 60
  Synthetic construct

SEQ ID NO: 75 - protein, 186 aa, Artificial Sequence
  Synthetic construct
LSQEKIIGWFQGRMEFGPRALGGRSIIGDPRSTTMQATMNLKIKFRESFRPFAPAVMREHVTEYFDIDSDSPYMLQVAPVVENRRRKMSDEEEKLFGIAKLNVPKSDIPAVTHVDYSARIQTVSSTQHPTFYKLLSAFKAKTGCPVLVNTSFNVRGEPIVCTPAEAYACFMRTNMDVLVVGNLVML

SEQ ID NO: 76 - protein, 36 aa, Artificial Sequence
  Synthetic construct
FNVKGQPIVNSPIEAIRSFQSTNIDVLIIGDFIAEK

SEQ ID NO: 77 - protein, 139 aa, Artificial Sequence
  Synthetic construct
AEKLEQGKIVGWFQGRSEFGPRALGNRSILTKPYPAEMKDYLNSRVKFREAFRPFAPAVLAEHVNEYFDINQESPHMLIACQAQVNNAHKIAATVHVDNSCRVQTVSRMDNQRFRKLLEAFFELTGCPVLLNTSFNVKG

SEQ ID NO: 78 - protein, 171 aa, Artificial Sequence
  Synthetic construct
AELLARGEVVARVSGREEFGARSLGNRAILADPSRLDVAKVVNKAIKCRDFWMPFAPSVMAEHADRHVRNPKNLTAPYMILAFDSQNTEEVRAACHPEDATIRPQIVTRENNASYHRVIEEFHRRTGRGTILNTSLNIHGEPIVSTPDDAISVMQRSGLHYLALGPYLIAK

SEQ ID NO: 79 - protein, 164 aa, Artificial Sequence
  Synthetic construct
VIAWFYGKSEYGPRALGNRSILANPGWLDIKDIINEKIKFRELFRPFAPAVLEEDATHYFDFKKTELCYYMLQTANVIPQQAEKIAGVVHADGTARIQLVSENINPVFHQLISAFKNKSGISCLLNTSFNTSGQPLINDPETAIETFARTELDYLFIEGYVIWK

SEQ ID NO: 80 - protein, 118 aa, Artificial Sequence
  Synthetic construct
SSPYMLLVQPVVKSRRKPLPEGYNSMSLKEKLYHLRSDMPAITHIDFSARIQTVHKETNPRYYQLIESFKKLTGYGVLVNTSFNVRGEPIICTPEDAYRCFMRTEMDFLVVGNYVFDK

SEQ ID NO: 81 - protein, 187 aa, Artificial Sequence; identical to SEQ ID NO: 61
  Synthetic construct

SEQ ID NO: 82 - protein, 157 aa, Artificial Sequence
  Synthetic construct
AENLAEGKVVGWLQGRMEFGPRALGGRSILGDARNTKMQSVMNLKIKYRESFRPFAPSVVRERVSDFFEMNTDSPYMLLVAPVQAKRCLPLPAQHAALWGIDLLNVPRSDIPAVTHLDYSARIQTVHEDTNPRYYRLLKAFEAQTGYPVLVNTSFNV

SEQ ID NO: 83 - protein, 58 aa, Artificial Sequence
  Synthetic construct
PRALGFRSILARPDSIKIRDRINKIKNREYWRPLAPVILEEELKNYFKTDFISPFMLF

SEQ ID NO: 84 - protein, 13 aa, Artificial Sequence
  Synthetic construct
EVGPRALCNTSLL

SEQ ID NO: 85 - protein, 13 aa, Artificial Sequence
  Synthetic construct
EVGPRALCNTTTL

SEQ ID NO: 86 - protein, 13 aa, Artificial Sequence
  Synthetic construct
EVGPRALCHTSTL

SEQ ID NO: 87 - protein, 13 aa, Artificial Sequence
  Synthetic construct
EFGPRALGNRSIL

SEQ ID NO: 88 - protein, 13 aa, Artificial Sequence
  Synthetic construct
  MISC_FEATURE: Xaa = U
  misc_feature (2)..(2): Xaa can be any naturally occurring amino acid
EXGPRALGHRTIF

SEQ ID NO: 89 - protein, 13 aa, Artificial Sequence
  Synthetic construct
ETFARALGNRSVI

SEQ ID NO: 90 - protein, 12 aa, Artificial Sequence
  Synthetic construct
RVKDREWWRPFA

SEQ ID NO: 91 - protein, 13 aa, Artificial Sequence
  Synthetic construct
RMINNRNTVMPVA

SEQ ID NO: 92 - protein, 12 aa, Artificial Sequence
  Synthetic construct
RMNDRTNEMPFE

SEQ ID NO: 93 - protein, 12 aa, Artificial Sequence
  Synthetic construct
TMNNRNTVMPMA

SEQ ID NO: 94 - protein, 13 aa, Artificial Sequence
  Synthetic construct
ELNDRTFVMPMAP

SEQ ID NO: 95 - protein, 12 aa, Artificial Sequence
  Synthetic construct
ANNRNTFMPMAI

SEQ ID NO: 96 - protein, 36 aa, Artificial Sequence
  Synthetic construct
  misc_feature (2)..(16): Xaa can be any naturally occurring amino acid
  misc_feature (18)..(18): Xaa can be any naturally occurring amino acid
  misc_feature (20)..(31): Xaa can be any naturally occurring amino acid
  misc_feature (33)..(35): Xaa can be any naturally occurring amino acid
NXXXXXXXXXXXXXXXTXTXXXXXXXXXXXXIXXXN

SEQ ID NO: 97 - protein, 5 aa, Artificial Sequence
  Synthetic construct
  misc_feature (1)..(2): Xaa can be any naturally occurring amino acid
  misc_feature (4)..(4): Xaa can be any naturally occurring amino acid
XXQXA

SEQ ID NO: 98 - protein, 22 aa, Artificial Sequence
  Synthetic construct
HSLGMFYSALTQYLGFPHFGDE

SEQ ID NO: 99 - protein, 22 aa, Artificial Sequence
  Synthetic construct
HSLGLLYSAVTYYTGFKVNTGE

SEQ ID NO: 100 - protein, 22 aa, Artificial Sequence
  Synthetic construct
QSIGHFYSTIGALAGMQGLHEE

SEQ ID NO: 101 - protein, 22 aa, Artificial Sequence
  Synthetic construct
HSLGLLYSAVTYFCGFKVNSGE

SEQ ID NO: 102 - protein, 22 aa, Artificial Sequence
  Synthetic construct
HSLGMFYSVITQFIGFPYFGDE

SEQ ID NO: 103 - protein, 22 aa, Artificial Sequence
  Synthetic construct
HSIGLFYSSVTYFLGFKVNSDE

SEQ ID NO: 104 - protein, 22 aa, Artificial Sequence
  Synthetic construct
HSLGIFYSLITQYAGFTRDKDE

SEQ ID NO: 105 - protein, 22 aa, Artificial Sequence
  Synthetic construct
LNLGTPYRLLAITMPEVTSGRP

SEQ ID NO: 106 - protein, 22 aa, Artificial Sequence
  Synthetic construct
HSLGLFYAGLTAYLGFAINEGE

SEQ ID NO: 107 - protein, 22 aa, Artificial Sequence
  Synthetic construct
HSVGIFYTSFTQYLGFPKYGDE

SEQ ID NO: 108 - protein, 22 aa, Artificial Sequence
  Synthetic construct
HSLGLLYSAITYYTGFKVNSGE

SEQ ID NO: 109 - protein, 22 aa, Artificial Sequence
  Synthetic construct
HSLGLLYSAFTYYCGFKVNSGE

SEQ ID NO: 110 - protein, 22 aa, Artificial Sequence
  Synthetic construct
HTLLLLVKSFTEIYKLGSTVDD

SEQ ID NO: 111 - protein, 22 aa, Artificial Sequence
  Synthetic construct
DSLGGLYGALTEYLGFEMLDGE

SEQ ID NO: 112 - protein, 22 aa, Artificial Sequence
  Synthetic construct
HSLGLLYSAFTYYTGFTVNSGE

SEQ ID NO: 113 - protein, 22 aa, Artificial Sequence
  Synthetic construct
QSLGMFYSMFTQLCGFTRDADE

SEQ ID NO: 114 - protein, 22 aa, Artificial Sequence
  Synthetic construct
HSIGLLYSAFTQFLGFKVNSGE

SEQ ID NO: 115 - protein, 22 aa, Artificial Sequence
  Synthetic construct
HSLGLLYSTFTTYAGFKVNSGE

SEQ ID NO: 116 - protein, 22 aa, Artificial Sequence
  Synthetic construct
HSLGLLYSAFTYYTGFKVNSGE

SEQ ID NO: 117 - protein, 22 aa, Artificial Sequence
  Synthetic construct
CDLARIYKITTLILGMKPDEHE

SEQ ID NO: 118 - protein, 22 aa, Artificial Sequence
  Synthetic construct
HSIGLLYSAVTYHCGFKVNSGE

SEQ ID NO: 119 - protein, 22 aa, Artificial Sequence
  Synthetic construct
HTLLLLVKSFTEIYKLGSTVDD

SEQ ID NO: 120 - protein, 22 aa, Artificial Sequence
  Synthetic construct
HSIGLLYSAVTYHCGFKVNSGE

SEQ ID NO: 121 - protein, 22 aa, Artificial Sequence
  Synthetic construct
DSLGYFYGRITKLLGYKPNQHE

SEQ ID NO: 122 - protein, 22 aa, Artificial Sequence
  Synthetic construct
HSVGIFYTSFTQYLGFPKYGDE

SEQ ID NO: 123 - protein, 22 aa, Artificial Sequence
  Synthetic construct
HSLGLLYSAFTYYLGFKVNSGE

SEQ ID NO: 124 - protein, 22 aa, Artificial Sequence
  Synthetic construct
HSLGLLYSAFTYFTGFRVNSGE

SEQ ID NO: 125 - protein, 22 aa, Artificial Sequence
  Synthetic construct
HSLGLLYSAITYYTGFKVNSGE

SEQ ID NO: 126 - protein, 22 aa, Artificial Sequence
  Synthetic construct
HSIGLLYSAFTYFTGFKVNSGE

SEQ ID NO: 127 - protein, 22 aa, Artificial Sequence
  Synthetic construct
NSLGLLYSAVTYFLGFKHHCDE

SEQ ID NO: 128 - protein, 22 aa, Artificial Sequence
  Synthetic construct
HTQLVLFRDHFDYVVLGRTRDD

SEQ ID NO: 129 - protein, 22 aa, Artificial Sequence
  Synthetic construct
HSVGNLWSVITALLSMVPLEHE

SEQ ID NO: 130 - protein, 22 aa, Artificial Sequence
  Synthetic construct
HSIGLLYSTITGYLGFKVNDGE

SEQ ID NO: 131 - protein, 22 aa, Artificial Sequence
  Synthetic construct
HSLGLLYSAFTYFLGFKVNSGE

SEQ ID NO: 132 - protein, 152 aa, Artificial Sequence
  Synthetic construct
AAELASVGFVNLVRGMEFGPRALCNTSTLAPTRDVVEINLRNDRTVMPMAPVMTESQADTLFETKDIDKVHKSLEYMITNYKGKAELLGGAHYYPDPLDVYTRPQTDDPNVHFMLKILDKFGGPPLINTSFNYHGVPIVLDEQIIHHFMESD

SEQ ID NO: 133 - protein, 419 aa, Artificial Sequence
  Synthetic construct
SVSYDFTHHDGHHKNSSAMVFAGDFKSKHHGGDKIHFLVVDGFGNGECLSIYANKDLFVDIGGKLLHRVHGYDKSLGLMYQYATAYLGMKMHQDEYKLLGYETRSGIPCPYDVNKSDSAFPKYAIEWIDKMYKSLPKYVGHWFNNGNLTDCKYWDEKYILQFVKDPRCIKSLNVDFTHHDSHRVAAECFYKNYSQQLLDENDYCIVADGFGTFGESLSIYGYHNGKAHVKSRVFDYINSLGLLYQYATLYLGMKMHNHEYKMLGYEAHIHEHFTQDEIFIMDGWADLQTDKILKSMNSVSLKQTDPYDVAGLEALNAWFYHLERIYRLPPNLDLYDVLKRLTFSDDVIEDRVIIAYYVQRVLESVVLKLKSDLYKPKNLILSGGVFYNVKLNNILDKSIGKFCIMPLAGDQGAALGAAA

SEQ ID NO: 134 - protein, 201 aa, Artificial Sequence
  Synthetic construct
ALLASGKVVGWFQGRMEFGPRALGNRSILADPRSPDMQDTINLKIKFRESFRPFAPSVLREVSDFETKDIDEVDKSSPYMLLADVKEGRRIPMTEEEALFGIDKLNVRVNIPRSDIPAVTHVDPKLDVYTARPQTVDDETNPRYHKLISAFKETGCPVLINTSFNVRGEPIVCSPEDALRCFMRTDMDLVLGNYILKDLRH

SEQ ID NO: 135 - protein, 22 aa, Artificial Sequence
  Synthetic construct
HSLGLLYSAATYYLGFKVNSDE